SEQUENCE LISTING (239 sequences)

SEQ ID NO: 1 - protein, 7 aa, Artificial Sequence
  Tyrosinase-related protein (TYRP)-1 intracellular retention signal
NQPLLTD

SEQ ID NO: 2 - protein, 15 aa, Artificial Sequence
  Adenoviral E3/19K intracellular retention signal
KYKSRRSFIDEKKMP

SEQ ID NO: 3 - protein, 6 aa, Artificial Sequence
  Adenoviral E3/19K intracellular retention signal
DEKKMP

SEQ ID NO: 4 - protein, 1350 aa, Artificial Sequence [sequence omitted]

SEQ ID NO: 5 - protein, 1717 aa, Artificial Sequence [sequence omitted]

SEQ ID NO: 6 - protein, 36 aa, Artificial Sequence
  nucleic acid construct comprising a tyrp-1 retention protein sequence
RARRSMDEANQPLLTDQYQCYAEEYEKLQNPNQSVV

SEQ ID NO: 7 - protein, 234 aa, Artificial Sequence
  spacer sequence, hinge-CH2CH3 of human IgG1
AEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKD

SEQ ID NO: 8 - protein, 46 aa, Artificial Sequence
  spacer sequence, human CD8 stalk
TTTPAPRPPTPAPTIASQPLSLRPEACRPAAGGAVHTRGLDFACDI

SEQ ID NO: 9 - protein, 20 aa, Artificial Sequence
  spacer sequence, human IgG1 hinge
AEPKSPDKTHTCPPCPKDPK

SEQ ID NO: 10 - protein, 185 aa, Artificial Sequence
  spacer sequence, CD2 ectodomain
KEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLD

SEQ ID NO: 11 - protein, 259 aa, Artificial Sequence
  spacer sequence, CD34 ectodomain
SLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKT

SEQ ID NO: 12 - protein, 140 aa, Artificial Sequence
  comprising CD28 transmembrane domain and CD3 Z endodomain
FWVLVVVGGVLACYSLLVTVAFIIFWVRRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 180 aa, Artificial Sequence
  comprising CD28 transmembrane domain and CD28 and CD3 Zeta endodomains
FWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 216 aa, Artificial Sequence
  comprising CD28 transmembrane domain and CD28, OX40 and CD3 Zeta endodomains
FWVLVVVGGVLACYSLLVTVAFIIFWVRSKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 729 aa, Artificial Sequence
  CD45 trans-membrane and endodomain sequence
ALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 16 - protein, 362 aa, Artificial Sequence
  CD148 trans-membrane and endodomain sequence
AVFGCIFGALVIVTVGGFIFWRKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 17 - protein, 136 aa, Artificial Sequence
  marker/suicide polypeptide
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 18 - protein, 8 aa, Artificial Sequence
  cleavage site
DMESNPGP

SEQ ID NO: 19 - protein, 8 aa, Artificial Sequence
  cleavage site
DVELNPGP

SEQ ID NO: 20 - protein, 8 aa, Artificial Sequence
  cleavage site
DVEENPGP

SEQ ID NO: 21 - protein, 8 aa, Artificial Sequence
  cleavage site
DIELNPGP

SEQ ID NO: 22 - protein, 8 aa, Artificial Sequence
  cleavage site
DIEQNPGP

SEQ ID NO: 23 - protein, 8 aa, Artificial Sequence
  cleavage site
DSEFNPGP

SEQ ID NO: 24 - protein, 19 aa, Artificial Sequence
  cleavage sequence
LLNFDLLKLAGDVESNPGP

SEQ ID NO: 25 - protein, 19 aa, Artificial Sequence
  cleavage sequence
LLNFDLLKLAGDVQSNPGP

SEQ ID NO: 26 - protein, 19 aa, Artificial Sequence
  cleavage sequence
LLNFDLLKLAGDVEINPGP

SEQ ID NO: 27 - protein, 19 aa, Artificial Sequence
  cleavage sequence
LLNFDLLKLAGDVEFNPGP

SEQ ID NO: 28 - protein, 19 aa, Artificial Sequence
  cleavage sequence
LLNFDLLKLAGDVESHPGP

SEQ ID NO: 29 - protein, 19 aa, Artificial Sequence
  cleavage sequence
LLNFDLLKLAGDVESEPGP

SEQ ID NO: 30 - protein, 19 aa, Artificial Sequence
  cleavage sequence
LLNFDLLKLAGDVESQPGP

SEQ ID NO: 31 - protein, 19 aa, Artificial Sequence
  cleavage sequence
LLNFDLLKLAGDVESNPGG

SEQ ID NO: 32 - protein, 20 aa, Artificial Sequence
  2A-like sequence
YHADYYKQRLIHDVEMNPGP

SEQ ID NO: 33 - protein, 20 aa, Artificial Sequence
  2A-like sequence
HYAGYFADLLIHDIETNPGP

SEQ ID NO: 34 - protein, 20 aa, Artificial Sequence
  2A-like sequence
QCTNYALLKLAGDVESNPGP

SEQ ID NO: 35 - protein, 19 aa, Artificial Sequence
  2A-like sequence
ATNFSLLKQAGDVEENPGP

SEQ ID NO: 36 - protein, 19 aa, Artificial Sequence
  2A-like sequence
AARQMLLLLSGDVETNPGP

SEQ ID NO: 37 - protein, 20 aa, Artificial Sequence
  2A-like sequence
RAEGRGSLLTCGDVEENPGP

SEQ ID NO: 38 - protein, 20 aa, Artificial Sequence
  2A-like sequence
TRAEIEDELIRAGIESNPGP

SEQ ID NO: 39 - protein, 20 aa, Artificial Sequence
  2A-like sequence
TRAEIEDELIRADIESNPGP

SEQ ID NO: 40 - protein, 20 aa, Artificial Sequence
  2A-like sequence
AKFQIDKILISGDVELNPGP

SEQ ID NO: 41 - protein, 20 aa, Artificial Sequence
  2A-like sequence
SSIIRTKMLVSGDVEENPGP

SEQ ID NO: 42 - protein, 20 aa, Artificial Sequence
  2A-like sequence
CDAQRQKLLLSGDIEQNPGP

SEQ ID NO: 43 - protein, 20 aa, Artificial Sequence
  2A-like sequence
YPIDFGGFLVKADSEFNPGP

SEQ ID NO: 44 - protein, 57 aa, Artificial Sequence
  cleavage sequence
VTELLYRMKRAETYCPRPLAIHPTEARHKQKIVAPVKQTLNFDLLKLAGDVESNPGP

SEQ ID NO: 45 - protein, 40 aa, Artificial Sequence
  cleavage sequence
LLAIHPTEARHKQKIVAPVKQTLNFDLLKLAGDVESNPGP

SEQ ID NO: 46 - protein, 33 aa, Artificial Sequence
  cleavage sequence
EARHKQKIVAPVKQTLNFDLLKLAGDVESNPGP

SEQ ID NO: 47 - protein, 24 aa, Artificial Sequence
  cleavage sequence
APVKQTLNFDLLKLAGDVESNPGP

SEQ ID NO: 48 - protein, 5 aa, Artificial Sequence
  linker
SGGGS

SEQ ID NO: 49 - protein, 4 aa, Artificial Sequence
  intracellular retention signal
  misc_feature (4)..(4): Xaa can be any naturally occurring amino acid
NPFX

SEQ ID NO: 50 - protein, 4 aa, Artificial Sequence
  intracellular retention signal
KDEL

SEQ ID NO: 51 - protein, 4 aa, Artificial Sequence
  intracellular retention signal
  misc_feature (3)..(4): Xaa can be any naturally occurring amino acid
KKXX

SEQ ID NO: 52 - protein, 5 aa, Artificial Sequence
  intracellular retention signal
  misc_feature (2)..(2): Xaa can be any naturally occurring amino acid
  misc_feature (4)..(5): Xaa can be any naturally occurring amino acid
KXKXX

SEQ ID NO: 53 - protein, 4 aa, Artificial Sequence
  intracellular retention signal
  misc_feature (3)..(3): Xaa can be any naturally occurring amino acid
NPXY

SEQ ID NO: 54 - protein, 4 aa, Artificial Sequence
  intracellular retention signal
  misc_feature (2)..(3): Xaa can be any naturally occurring amino acid
  MISC_FEATURE (4)..(4): Xaa is an amino acid with a bulky hydrophobic side chain
YXXX

SEQ ID NO: 55 - protein, 8 aa, Artificial Sequence
  intracellular retention signal
  misc_feature (3)..(5): Xaa can be any naturally occurring amino acid
DEXXXLLI

SEQ ID NO: 56 - protein, 5 aa, Artificial Sequence
  intracellular retention signal
  misc_feature (2)..(3): Xaa can be any naturally occurring amino acid
DXXLL

SEQ ID NO: 57 - protein, 4 aa, Artificial Sequence
  intracellular retention signal
DPFW

SEQ ID NO: 58 - protein, 5 aa, Artificial Sequence
  intracellular retention signal
  misc_feature (2)..(2): Xaa can be any naturally occurring amino acid
  misc_feature (4)..(4): Xaa can be any naturally occurring amino acid
FXDXF

SEQ ID NO: 59 - protein, 6 aa, Artificial Sequence
  intracellular retention signal
  MISC_FEATURE (2)..(2): Xaa is an amino acid with a bulky hydrophobic side chain
  misc_feature (3)..(3): Xaa can be any naturally occurring amino acid
  MISC_FEATURE (4)..(4): Xaa is an amino acid with a bulky hydrophobic side chain
LXXXDE

SEQ ID NO: 60 - protein, 5 aa, Artificial Sequence
  intracellular retention signal
LLDLL

SEQ ID NO: 61 - protein, 5 aa, Artificial Sequence
  intracellular retention signal
PWDLW

SEQ ID NO: 62 - protein, 20 aa, Mus musculus
METDTLILWVLLLLVPGSTG

SEQ ID NO: 63 - protein, 21 aa, Artificial Sequence
  signal peptide derived from Human CD8a
MSLPVTALLLPLALLLHAARP

SEQ ID NO: 64 - protein, 248 aa, Artificial Sequence
  scFv aCD19
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITKAGGGGSGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 65 - protein, 47 aa, Homo sapiens
PTTTPAPRPPTPAPTIASQPLSLRPEACRPAAGGAVHTRGLDFACDI

SEQ ID NO: 66 - protein, 27 aa, Homo sapiens
FWVLVVVGGVLACYSLLVTVAFIIFWV

SEQ ID NO: 67 - protein, 113 aa, Homo sapiens
RRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 68 - protein, 20 aa, Artificial Sequence
  2A peptide
RAEGRGSLLTCGDVEENPGP

SEQ ID NO: 69 - protein, 18 aa, Artificial Sequence
  signal peptide derived from mouse Ig kappa
MAVPTQVLGLLLLWLTDA

SEQ ID NO: 70 - protein, 254 aa, Artificial Sequence
  scFv aCD33
RCDIQMTQSPSSLSASVGDRVTITCRASEDIYFNLVWYQQKPGKAPKLLIYDTNRLADGVPSRFSGSGSGTQYTLTISSLQPEDFATYYCQHYKNYPLTFGQGTKLEIKRSGGGGSGGGGSGGGGSGGGGSRSEVQLVESGGGLVQPGGSLRLSCAASGFTLSNYGMHWIRQAPGKGLEWVSSISLNGGSTYYRDSVKGRFTISRDNAKSTLYLQMNSLRAEDTAVYYCAAQDAYTGGYFDYWGQGTLVTVSSM

SEQ ID NO: 71 - protein, 231 aa, Artificial Sequence
  hinge and Fc derived from human IgG1
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 4 aa, Artificial Sequence
  linker
KDPK

SEQ ID NO: 73 - protein, 21 aa, Homo sapiens
AVFGCIFGALVIVTVGGFIFW

SEQ ID NO: 74 - protein, 341 aa, Homo sapiens
RKKRKDAKNNEVSFSQIKPKKSKLIRVENFEAYFKKQQADSNCGFAEEYEDLKLVGISQPKYAAELAENRGKNRYNNVLPYDISRVKLSVQTHSTDDYINANYMPGYHSKKDFIATQGPLPNTLKDFWRMVWEKNVYAIIMLTKCVEQGRTKCEEYWPSKQAQDYGDITVAMTSEIVLPEWTIRDFTVKNIQTSESHPLRQFHFTSWPDHGVPDTTDLLINFRYLVRDYMKQSPPESPILVHCSAGVGRTGTFIAIDRLIYQIENENTVDVYGIVYDLRMHRPLMVQTEDQYVFLNQCVLDIVRSQKDSKVDLIYQNTTAMTIYENLAPVTTFGKTNGYIA

SEQ ID NO: 75 - protein, 22 aa, Homo sapiens
ALIAFLAFLIIVTSIALLVVLY

SEQ ID NO: 76 - protein, 4 aa, Mus musculus
METD

SEQ ID NO: 77 - protein, 11 aa, Mus musculus
TLILWVLLLLV

SEQ ID NO: 78 - protein, 5 aa, Mus musculus
PGSTG

SEQ ID NO: 79 - protein, 25 aa, Homo sapiens
MNRGVPFRHLLLVLQLALLPAATQG

SEQ ID NO: 80 - protein, 23 aa, Homo sapiens
MYLWLKLLAFGFAFLDTEVFVTG

SEQ ID NO: 81 - protein, 21 aa, Homo sapiens
MALPVTALLLPLALLLHAARP

SEQ ID NO: 82 - protein, 21 aa, Homo sapiens
MRPRLWLLLAAQLTVLHGNSV

SEQ ID NO: 83 - protein, 24 aa, Homo sapiens
MSFPCKFVASFLLIFNVSSKGAVS

SEQ ID NO: 84 - protein, 24 aa, Homo sapiens
MPMGSLQPLATLYLLGMLVASCLG

SEQ ID NO: 85 - protein, 25 aa, Homo sapiens
MAGPPRLLLLPLLLALARGLPGALA

SEQ ID NO: 86 - protein, 24 aa, Homo sapiens
MSAPKLLSLGCIFFPLLLFQQARA

SEQ ID NO: 87 - protein, 19 aa, Homo sapiens
MRFFVPLFLVGILFPAILA

SEQ ID NO: 88 - protein, 23 aa, Homo sapiens
MPGFLVRILPLLLVLLLLGPTRG

SEQ ID NO: 89 - protein, 24 aa, Homo sapiens
MKRFLFLLLTISLLVMVQIQTGLS

SEQ ID NO: 90 - protein, 21 aa, Homo sapiens
MQGPPLLTAAHLLCVCTAALA

SEQ ID NO: 91 - protein, 22 aa, Homo sapiens
MGPTSGPSLLLLLLTHLPLALG

SEQ ID NO: 92 - protein, 18 aa, Homo sapiens
MQPILLLLAFLLLPRADA

SEQ ID NO: 93 - protein, 20 aa, Homo sapiens
MEAPAQLLFLLLLWLPDTTR

SEQ ID NO: 94 - protein, 20 aa, Homo sapiens
MVLQTQVFISLLLWISGAYG

SEQ ID NO: 95 - protein, 19 aa, Homo sapiens
MAWAPLLLTLLAHCTDCWA

SEQ ID NO: 96 - protein, 22 aa, Homo sapiens
MLKPSLPFTSLLFLQLPLLGVG

SEQ ID NO: 97 - protein, 20 aa, Homo sapiens
MAWASRLGLLLALLLPVVGA

SEQ ID NO: 98 - protein, 23 aa, Homo sapiens
MGVPRPQPWALGLLLFLLPGSLG

SEQ ID NO: 99 - protein, 4 aa, Artificial Sequence
  ITAM motif
  misc_feature (2)..(3): Xaa can be any naturally occurring amino acid
  MISC_FEATURE (4)..(4): Xaa may be Leu or Ile
YXXX

SEQ ID NO: 100 - protein, 4 aa, Artificial Sequence
  basic amino acid furin target sequence
  misc_feature (2)..(2): Xaa can be any naturally occurring amino acid
  MISC_FEATURE (3)..(3): Xaa may be Arg or Lys
RXXR

SEQ ID NO: 101 - protein, 7 aa, Artificial Sequence
  consensus Tobacco Etch Virus (TEV) cleavage site
ENLYFQS

SEQ ID NO: 102 - protein, 8 aa, Artificial Sequence
  motif involved in cleavage activity
  misc_feature (2)..(2): Xaa can be any naturally occurring amino acid
  misc_feature (4)..(4): Xaa can be any naturally occurring amino acid
DXEXNPGP

SEQ ID NO: 103 - protein, 8 aa, Artificial Sequence
  cleavage site
  MISC_FEATURE (2)..(2): Xaa may be Ile, Val, Met or Ser
  MISC_FEATURE (4)..(4): Xaa may be Thr, Met, Ser, Leu, Glu, Gln or Phe
DXEXNPGP

SEQ ID NO: 104 - protein, 4 aa, Artificial Sequence
  C-terminal signal, motif of postassium channels
  misc_feature (2)..(2): Xaa can be any naturally occurring amino acid
RXRR

SEQ ID NO: 105 - protein, 12 aa, Homo sapiens
INFDNPVYQKTT

SEQ ID NO: 106 - protein, 12 aa, Homo sapiens
VEIGNPTYKMYE

SEQ ID NO: 107 - protein, 12 aa, Homo sapiens
TNFTNPVYATLY

SEQ ID NO: 108 - protein, 12 aa, Drosophila sp.
GNFANPVYESMY

SEQ ID NO: 109 - protein, 12 aa, Caenorhabditis elegans
TTFTNPVYELED

SEQ ID NO: 110 - protein, 12 aa, Caenorhabditis elegans
LRVDNPLYDPDS

SEQ ID NO: 111 - protein, 12 aa, Homo sapiens
IIFENPMYSARD

SEQ ID NO: 112 - protein, 12 aa, Homo sapiens
TNFENPIYAQME

SEQ ID NO: 113 - protein, 12 aa, Homo sapiens
DTGENPIYKSAV

SEQ ID NO: 114 - protein, 11 aa, Homo sapiens
TTVVNPKYEGK

SEQ ID NO: 115 - protein, 12 aa, Drosophila sp.
WDTENPIYKQAT

SEQ ID NO: 116 - protein, 11 aa, Drosophila sp.
STFKNPMYAGK

SEQ ID NO: 117 - protein, 12 aa, Homo sapiens
HGYENPTYRFLE

SEQ ID NO: 118 - protein, 12 aa, Homo sapiens
NGYENPTYKFFE

SEQ ID NO: 119 - protein, 12 aa, Drosophila sp.
NGYENPTYKYFE

SEQ ID NO: 120 - protein, 12 aa, Homo sapiens
YASSNPEYLSAS

SEQ ID NO: 121 - protein, 12 aa, Homo sapiens
GSVQNPVYHNQP

SEQ ID NO: 122 - protein, 12 aa, Homo sapiens
TAVGNPEYLNTV

SEQ ID NO: 123 - protein, 12 aa, Homo sapiens
ISLDNPDYQQDF

SEQ ID NO: 124 - protein, 11 aa, Homo sapiens
RKRSHAGYQTI

SEQ ID NO: 125 - protein, 10 aa, Homo sapiens
KHHHAGYEQF

SEQ ID NO: 126 - protein, 11 aa, Gallus gallus
KKHHNTGYEQF

SEQ ID NO: 127 - protein, 11 aa, Gallus gallus
RRKSRTGYQSV

SEQ ID NO: 128 - protein, 11 aa, Gallus gallus
RRKSYAGYQTL

SEQ ID NO: 129 - protein, 12 aa, Drosophila sp.
RRRSTSRGYMSF

SEQ ID NO: 130 - protein, 11 aa, Lumbricus terrestris
RKRSRRGYESV

SEQ ID NO: 131 - protein, 10 aa, Homo sapiens
KSIRSGYEVM

SEQ ID NO: 132 - protein, 12 aa, Homo sapiens
HCGGPRPGYETL

SEQ ID NO: 133 - protein, 12 aa, Mus musculus
HCRTRRAEYETL

SEQ ID NO: 134 - protein, 10 aa, Homo sapiens
RRRPSAYQAL

SEQ ID NO: 135 - protein, 9 aa, Homo sapiens
RRRSYQNIP

SEQ ID NO: 136 - protein, 10 aa, Homo sapiens
KKHCSYQDIL

SEQ ID NO: 137 - protein, 10 aa, Mus musculus
RRRSAYQDIR

SEQ ID NO: 138 - protein, 11 aa, Rattus norvegicus
RKRRRSYQDIM

SEQ ID NO: 139 - protein, 13 aa, Rattus norvegicus
KFCKSKERNYHTL

SEQ ID NO: 140 - protein, 14 aa, Drosophila sp.
KFYKARNERNYHTL

SEQ ID NO: 141 - protein, 14 aa, Homo sapiens
KIRLRCQSSGYQRI

SEQ ID NO: 142 - protein, 14 aa, Mus musculus
KIRQRHQSSAYQRI

SEQ ID NO: 143 - protein, 15 aa, Homo sapiens
HFCLYRKRPGYDQLN

SEQ ID NO: 144 - protein, 11 aa, Homo sapiens
SLSRGSGYKEI

SEQ ID NO: 145 - protein, 13 aa, Homo sapiens
RRLRKGYTPLMET

SEQ ID NO: 146 - protein, 14 aa, Homo sapiens
RRAGHSSYTPLPGS

SEQ ID NO: 147 - protein, 18 aa, Dictyostelium sp.
KKLRQQKQQGYQAIINNE

SEQ ID NO: 148 - protein, 15 aa, Dictyostelium sp.
RSKSNQNQSYNLIQL

SEQ ID NO: 149 - protein, 17 aa, Dictyostelium sp.
RKTFYNNNQYNGYNIIN

SEQ ID NO: 150 - protein, 10 aa, Homo sapiens
PLSYTRFSLA

SEQ ID NO: 151 - protein, 11 aa, Homo sapiens
MTKEYQDLQHL

SEQ ID NO: 152 - protein, 10 aa, Homo sapiens
SYKYSKVNKE

SEQ ID NO: 153 - protein, 10 aa, Homo sapiens
PAAYRGVGDD

SEQ ID NO: 154 - protein, 10 aa, Homo sapiens
TGVYVKMPPT

SEQ ID NO: 155 - protein, 10 aa, Homo sapiens
LISYKGLPPE

SEQ ID NO: 156 - protein, 11 aa, Rattus norvegicus
ASDYQRLNLKL

SEQ ID NO: 157 - protein, 11 aa, Human immunodeficiency virus type 1
RQGYSPLSFQT

SEQ ID NO: 158 - protein, 19 aa, Homo sapiens
RMQAQPPGYRHVADGEDHA

SEQ ID NO: 159 - protein, 7 aa, Artificial Sequence
  Dileucine-based sorting signal
  MISC_FEATURE (1)..(1): Xaa may be Asp or Glu
  misc_feature (2)..(4): Xaa can be any naturally occurring amino acid
  MISC_FEATURE (7)..(7): Xaa may be Leu or Ile
XXXXLLX

SEQ ID NO: 160 - protein, 9 aa, Homo sapiens
SDKQTLLPN

SEQ ID NO: 161 - protein, 9 aa, Rattus norvegicus
DERAPLIRT

SEQ ID NO: 162 - protein, 9 aa, Homo sapiens
QEKDPLLKN

SEQ ID NO: 163 - protein, 9 aa, Coturnix sp.
TERNPLLKS

SEQ ID NO: 164 - protein, 9 aa, Homo sapiens
GENSPLLSG

SEQ ID NO: 165 - protein, 9 aa, Homo sapiens
EEKQPLLME

SEQ ID NO: 166 - protein, 9 aa, Oryzias latipes
GERQPLLQS

SEQ ID NO: 167 - protein, 9 aa, Gallus gallus
PEIQPLLTE

SEQ ID NO: 168 - protein, 9 aa, Carassius auratus
EGRQPLLGD

SEQ ID NO: 169 - protein, 9 aa, Homo sapiens
EANQPLLTD

SEQ ID NO: 170 - protein, 9 aa, Gallus gallus
ELHQPLLTD

SEQ ID NO: 171 - protein, 9 aa, Danio rerio
REFEPLLNA

SEQ ID NO: 172 - protein, 9 aa, Homo sapiens
EEKMAILMD

SEQ ID NO: 173 - protein, 9 aa, Homo sapiens
EEKLAILSQ

SEQ ID NO: 174 - protein, 9 aa, Mus musculus
SERDVLLDE

SEQ ID NO: 175 - protein, 9 aa, Homo sapiens
SERRNLLED

SEQ ID NO: 176 - protein, 9 aa, Rattus norvegicus
DDSGDLLPG

SEQ ID NO: 177 - protein, 9 aa, Homo sapiens
SQIKRLLSE

SEQ ID NO: 178 - protein, 9 aa, Felis catus
SHIKRLLSE

SEQ ID NO: 179 - protein, 9 aa, Mus musculus
RRTPSLLEQ

SEQ ID NO: 180 - protein, 9 aa, Homo sapiens
HRTPSLLEQ

SEQ ID NO: 181 - protein, 10 aa, Rattus norvegicus
EPRGSRLLVR

SEQ ID NO: 182 - protein, 19 aa, Homo sapiens
MDDQRDLISNNEQLPMLGR

SEQ ID NO: 183 - protein, 19 aa, Mus musculus
MDDQRDLISNHEQLPILGN

SEQ ID NO: 184 - protein, 20 aa, Gallus gallus
MAEEQRDLISSDGSSGVLPI

SEQ ID NO: 185 - protein, 23 aa, Danio rerio
MEPDHQNESLIQRVPSAETILGR

SEQ ID NO: 186 - protein, 23 aa, Danio rerio
MSSEGNETPLISDQSSVNMGPQP

SEQ ID NO: 187 - protein, 24 aa, Trypanosoma sp.
RPRRRTEEDELLPEEAEGLIDPQN

SEQ ID NO: 188 - protein, 19 aa, Homo sapiens
PDKHSLLVGDFREDDDTAL

SEQ ID NO: 189 - protein, 9 aa, Homo sapiens
TERERLLNF

SEQ ID NO: 190 - protein, 8 aa, Homo sapiens
VETDDLIL

SEQ ID NO: 191 - protein, 7 aa, Caenorhabditis elegans
FENDSLL

SEQ ID NO: 192 - protein, 9 aa, Saccharomyces cerevisiae
NEQSPLLHN

SEQ ID NO: 193 - protein, 8 aa, Saccharomyces cerevisiae
SEQTRLVP

SEQ ID NO: 194 - protein, 8 aa, Saccharomyces cerevisiae
EVDLDLLK

SEQ ID NO: 195 - protein, 13 aa, Homo sapiens
SFHDDSDEDLLHI

SEQ ID NO: 196 - protein, 13 aa, Bos taurus
TFHDDSDEDLLHV

SEQ ID NO: 197 - protein, 13 aa, Oryctolagus cuniculus
SFHDDSDEDLLNI

SEQ ID NO: 198 - protein, 13 aa, Gallus gallus
SFHDDSDEDLLNV

SEQ ID NO: 199 - protein, 13 aa, Homo sapiens
EESEERDDHLLPM

SEQ ID NO: 200 - protein, 13 aa, Gallus gallus
DESEERDDHLLPM

SEQ ID NO: 201 - protein, 12 aa, Homo sapiens
GYHDDSDEDLLE

SEQ ID NO: 202 - protein, 13 aa, Homo sapiens
ITGFSDDVPMVIA

SEQ ID NO: 203 - protein, 13 aa, Hydra sp.
INRFSDDEPLVVA

SEQ ID NO: 204 - protein, 13 aa, Homo sapiens
MLEASDDEALLVC

SEQ ID NO: 205 - protein, 13 aa, Homo sapiens
KNETSDDEALLLC

SEQ ID NO: 206 - protein, 12 aa, Mus musculus
WVVEAEDEPLLA

SEQ ID NO: 207 - protein, 12 aa, Homo sapiens
WVAEAEDEPLLT

SEQ ID NO: 208 - protein, 12 aa, Homo sapiens
HDDFADDISLLK

SEQ ID NO: 209 - protein, 13 aa, Mus musculus
GRDSPEDHSLLVN

SEQ ID NO: 210 - protein, 12 aa, Peromyscus maniculatus
VRCHPEDDRLLG

SEQ ID NO: 211 - protein, 13 aa, Homo sapiens
HRVSQDDLDLLTS

SEQ ID NO: 212 - protein, 13 aa, Homo sapiens
ASVSLLDDELMSL

SEQ ID NO: 213 - protein, 13 aa, Homo sapiens
ASSGLDDLDLLGK

SEQ ID NO: 214 - protein, 13 aa, Homo sapiens
VQNPSADRNLLDL

SEQ ID NO: 215 - protein, 13 aa, Homo sapiens
NALSWLDEELLCL

SEQ ID NO: 216 - protein, 13 aa, Drosophila sp.
TVDSIDDVPLLSD

SEQ ID NO: 217 - protein, 13 aa, Mus musculus
QEECPSDSEEDEG

SEQ ID NO: 218 - protein, 13 aa, Mus musculus
RDRDYDEDDEDDI

SEQ ID NO: 219 - protein, 15 aa, Mus musculus
LDETEDDELEYDDES

SEQ ID NO: 220 - protein, 10 aa, Homo sapiens
KDPDEVETES

SEQ ID NO: 221 - protein, 13 aa, Homo sapiens
HEFQDETDTEEET

SEQ ID NO: 222 - protein, 14 aa, Homo sapiens
QEKEDDGSESEEEY

SEQ ID NO: 223 - protein, 9 aa, Homo sapiens
GEDEESESD

SEQ ID NO: 224 - protein, 12 aa, Homo sapiens
GEDSDEEPDHEE

SEQ ID NO: 225 - protein, 11 aa, Homo sapiens
LEDDSDEEEDF

SEQ ID NO: 226 - protein, 9 aa, Human cytomegalovirus
KDSDEEENV

SEQ ID NO: 227 - protein, 13 aa, Herpesvirus 3
FEDSESTDTEEEF

SEQ ID NO: 228 - protein, 9 aa, Human immunodeficiency virus type 1
LEAQEEEEV

SEQ ID NO: 229 - protein, 22 aa, Saccharomyces cerevisiae
ADDLESGLGAEDDLEQDEQLEG

SEQ ID NO: 230 - protein, 12 aa, Saccharomyces cerevisiae
TEIDESFEMTDF

SEQ ID NO: 231 - protein, 18 aa, Saccharomyces cerevisiae
TEPEEVEDFDFDLSDEDH

SEQ ID NO: 232 - protein, 15 aa, Saccharomyces cerevisiae
FEIEEDDVPTLEEEH

SEQ ID NO: 233 - protein, 707 aa, Homo sapiens
KIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 234 - protein, 8 aa, Artificial Sequence
  cleavage site
DIETNPGP

SEQ ID NO: 235 - protein, 8 aa, Artificial Sequence
  cleavage site
DVETNPGP

SEQ ID NO: 236 - protein, 8 aa, Artificial Sequence
  cleavage site
DVEMNPGP

SEQ ID NO: 237 - protein, 8 aa, Artificial Sequence
  cleavage site
DVESNPGP

SEQ ID NO: 238 - protein, 4 aa, Artificial Sequence
  ER signal peptide
KKFF

SEQ ID NO: 239 - protein, 4 aa, Artificial Sequence
  intracellular retention signal
HDEL

(19) United States
(12) Patent Application Publication
(10) Pub. No.: US 2018/0104321 A1
(43) Pub. Date: Apr. 19, 2018

(54) NUCLEIC ACID CONSTRUCT FOR EXPRESSING MORE THAN ONE CHIMERIC ANTIGEN RECEPTOR

(71) Applicant: UCL BUSINESS PLC, London (GB)

(72) Inventors: Martin Pulé, London (GB); Shaun Cordoba, London (GB)

(21) Appl. No.: 15/568,874

(22) PCT Filed: Apr. 26, 2016
(86) PCT No.: PCT/GB2016/051162

§ 371 Date: Oct. 24, 2017

(30) Foreign Application Priority Data
Apr. 27, 2015 (GB) 1507115.2

(51) Int. Cl. A61K 39/00; C07K 14/725; C07K 16/28

(52) U.S. Cl. CPC A61K 39/0011; C07K 14/7051; C07K 16/2803; C07K 2319/33; C07K 2317/622; C07K 2319/02; A61K 2039/5156

(57) ABSTRACT

The present invention provides a nucleic acid construct comprising the following structure: A-X-B in which X is a nucleic acid sequence which encodes a cleavage site; and A and B are nucleic acid sequences encoding a first and a second chimeric antigen receptor (CAR), each CAR comprising: (i) an antigen-binding domain; (ii) a spacer (iii) a trans-membrane domain; and (iv) an endodomain wherein the antigen binding domains of the first and second CARs bind to different antigens, wherein the spacer of the first CAR is different to the spacer of the second CAR and wherein one of the first or second CARs is an activating CAR comprising an activating endodomain and the other CAR is an inhibitory CAR comprising a ligation-off inhibitory endodomain; and wherein: (a) the first and/or second CAR comprises an intracellular retention signal; and/or (b) the signal peptide of the first or second CAR comprises one or more mutation(s) such that it has fewer hydrophobic amino acids.

### FIELD OF THE INVENTION

The present invention relates to constructs and approaches for expressing more than one chimeric antigen receptor (CAR) at the surface of a cell. The cell may be capable of specifically recognising a target cell, due to a differential pattern of expression (or non-expression) of two or more antigens by the target cell. The constructs of the invention enable modulation of the relative expression of the two or more CARs at the cell surface by a method involving co-expression of the CARs from a single vector.

### BACKGROUND TO THE INVENTION

A number of immunotherapeutic agents have been described for use in cancer treatment, including therapeutic monoclonal antibodies (mAbs), immunoconjugated mAbs, radioconjugated mAbs and bi-specific T-cell engagers.

Typically these immunotherapeutic agents target a single antigen: for instance, Rituximab targets CD20; Myelotarg targets CD33; and Alemtuzumab targets CD52.

However, it is relatively rare for the presence (or absence) of a single antigen effectively to describe a cancer, which can lead to a lack of specificity.

Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted, whilst targeting CD52 to treat chronic lymphocytic leukaemia, the entire lymphoid compartment is depleted, whilst targeting CD33 to treat acute myeloid leukaemia, the entire myeloid compartment is damaged etc.

The predicted problem of “on-target off-tumour” toxicity has been borne out by clinical trials. For example, an approach targeting ERBB2 caused death to a patient with colon cancer metastatic to the lungs and liver. ERBB2 is over-expressed in colon cancer in some patients, but it is also expressed on several normal tissues, including heart and normal vasculature.

For some cancers, targeting the presence of two cancer antigens may be more selective and therefore effective than targeting one. For example, B-chronic lymphocytic leukaemia (B-CLL) is a common leukaemia which is currently treated by targeting CD19. This treats the lymphoma but also depletes the entire B-cell compartment such that the treatment has a considerable toxic effect. B-CLL has an unusual phenotype in that CD5 and CD19 are co-expressed. By targeting only cells which express CD5 and CD19, it would be possible to considerably reduce on-target off-tumour toxicity.

There is thus a need for immunotherapeutic agents which are capable of more targeting to reflect the complex pattern of marker expression that is associated with many cancers.

Chimeric Antigen Receptors (CARs)

Chimeric antigen receptors are proteins which graft the specificity of a monoclonal antibody (mAb) to the effector function of a T-cell. Their usual form is that of a type I transmembrane domain protein with an antigen recognizing amino terminus, a spacer, a transmembrane domain all connected to a compound endodomain which transmits T-cell survival and activation signals (see FIG. 1A).

The most common form of these molecules are fusions of single-chain variable fragments (scFv) derived from monoclonal antibodies which recognize a target antigen, fused via a spacer and a trans-membrane domain to a signaling endodomain. Such molecules result in activation of the T-cell in response to recognition by the scFv of its target. When T cells express such a CAR, they recognize and kill target cells that express the target antigen. Several CARs have been developed against tumour associated antigens, and adoptive transfer approaches using such CAR-expressing T cells are currently in clinical trial for the treatment of various cancers.

However, the use of CAR-expressing T cells is also associated with on-target, off tumour toxicity. For example, a CAR-based approach targeting carboxy anyhydrase-IX (CAIX) to treat renal cell carcinoma resulted in liver toxicity which is thought to be caused by the specific attack on bile duct epithelial cells (Lamers et al (2013) Mol. Ther. 21:904-912).

Dual Targeting CAR Approaches

In order to address the problem of “on target, off tumour” toxicity, CAR T cells have been developed with dual antigen specificity. In the “dual targeting” approach, two complementary CARs are co-expressed in the same T-cell population, each directed to a distant tumour target and engineered to provide complementary signals.

Wikie et al (2012 J Clin Immunol 32:1059-1070) describe a dual targeting approach in which ErbB2- and MUC1-specific CARs are co-expressed. The ErbB2-specific CAR provided the CD3 signal only and the MUC1-specific CAR provided the CD28 co-stimulatory signal only. It was found that complementary signalling occurred in the presence of both antigens, leading to IL-2 production. However, IL-2 production was modest when compared to control CAR-engineered T cells in which signaling is delivered by a fused CD28+CD3 endodomain.

A similar approach was described by Kloss et al (2013 Nature Biotechnol. 31:71-75) in which a CD-19 specific CAR was used which provides a CD3-mediated activation signal in combination with a chimeric co-stimulatory receptor specific for PSMA. With this ‘co-CAR’ design, the CAR T-cell receives an activation signal when it encounters a target cell with one antigen, and a co-stimulatory signal when it encounters a target cell with the other antigen, and only receives both activatory and co-stimulatory signals upon encountering target cells bearing both antigens.

This represents an early attempt at restricting CAR activity to only a target cell bearing two antigens. This approach however is limited: although CAR T-cell activity will be greatest against targets expressing both antigens, CAR T-cells will still kill targets expressing only antigen recognized by the activatory CAR; further, co-stimulation results in prolonged effects on T-cells which last long after release of target cell. Hence, activity against single-antigen positive T-cells equal to that against double-positives might be possible for example in a situation where single-positive tissues are adjacent to, or in a migratory path from double positive tumour.

There is thus a need for improved CAR-based therapeutic approaches with reduced on-target off-tumour toxicity where T-cell activation is wholly restricted to target cells which express both antigens.

### DESCRIPTION OF THE FIGURES

FIG. 1: (a) Generalized architecture of a CAR: A binding domain recognizes antigen; the spacer elevates the binding domain from the cell surface; the trans-membrane domain anchors the protein to the membrane and the endodomain transmits signals. (b) to (d): Different generations and permutations of CAR endodomains: (b) initial designs transmitted ITAM signals alone through FcϵR1-γ or CD3ζ endodomain, while later designs transmitted additional (c) one or (d) two co-stimulatory signals in cis.

FIG. 2: Schematic Diagram Illustrating Logic Gates

The invention relates to engineering T-cells to respond to logical rules of target cell antigen expression. This is best illustrated with an imaginary FACS scatter-plot. Target cell populations express both, either or neither of antigens “A” and “B”. Different target populations (marked in red) are killed by T-cells transduced with a pair of CARs connected by different gates. With OR gated receptors, both single-positive and double-positive cells will be killed. With AND gated receptors, only double-positive target cells are killed. With AND NOT gating, double-positive targets are preserved while single-positive targets will be killed.

FIG. 3: Creation of Target Cell Populations

SupT1 cells were used as target cells. These cells were transduced to express either CD19, CD33 or both CD19 and CD33. Target cells were stained with appropriate antibodies and analysed by flow cytometry.

FIG. 4: Cartoon Showing Both Versions of the Cassette Used to Express Both AND Gates

Activating and inhibiting CARs were co-expressed once again using a FMD-2A sequence. Signal1 is a signal peptide derived from IgG1 (but can be any effective signal peptide). scFv1 is the single-chain variable segment which recognizes CD19 (but can be a scFv or peptide loop or ligand or in fact any domain which recognizes any desired arbitrary target). STK is the CD8 stalk but may be any non-bulky extracellular domain. CD28tm is the CD28 trans-membrane domain but can by any stable type I protein transmembrane domain and CD3Z is the CD3 Zeta endodomain but can be any endodomain which contains ITAMs. Signal2 is a signal peptide derived from CD8 but can be any effective signal peptide which is different in DNA sequence from signal1. scFv recognizes CD33 but as for scFv1 is arbitrary. HC2CH3 is the hinge-CH2-CH3 of human IgG1 but can be any bulky extracellular domain. CD45 and CD148 are the transmembrane and endodomains of CD45 and CD148 respectively but can be derived from any of this class of protein.

FIG. 5: Schematic Representation of the Protein Structure of Chimeric Antigen Receptors (CARs) for the AND Gates

The stimulatory CAR consisting of an N-terminal anti-CD19 scFv domain followed by the extracellular stalk region of human CD8, human CD28 transmembrane domain and human CD3 Zeta (CD247) intracellular domain. Two inhibitory CARs were tested. These consist of an N-terminal anti-CD33 scFv domain followed by the extracellular hinge, CH2 and CH3 (containing a pvaa mutation to reduce FcR binding) region of human IgG1 followed by the transmembrane and intracellular domain of either human CD148 or CD45. “S” depicts the presence of disulphide bonds.

FIG. 6: Co-Expression of Activation and Inhibitory CARs

BW5147 cells were used as effector cells and were transduced to express both the activation anti-CD19 CAR and one of the inhibitory anti-CD33 CARs. Effector cells were stained with CD19-mouse-Fc and CD33-rabbit-Fc and with appropriate secondary antibodies and analysed by flow cytometry.

FIG. 7: Functional Analysis of the AND Gates

Effector cells (5×10̂4 cells) expressing activation anti-CD19 CAR and the inhibitory anti-CD33 CAR with the A) CD148 or B) CXD45 intracellular domain were co-incubated with a varying number of target cells and IL-2 was analysed after 16 hours by ELISA. The graph displays the maximum IL-2 secretion from a chemical stimulation (PMA and lonomycin) of the effector cells alone and the background IL-2 from effector cells without any stimulus from three replicates.

FIG. 8: Dissection of AND Gate Function

A. The prototype AND gate is illustrated on the right and its function in response to CD19, CD33 single and CD19, CD33 double positive targets is shown on the left. B. The scFvs are swapped so the activating endodomain is triggered by CD33 and the inhibitory endodomain is activated by CD19. This AND gate remains functional despite this scFv swap. C. The CD8 mouse stalk replaced Fc in the spacer of the inhibitory CAR. With this modification, the gate fails to respond to either CD19 single positive or CD19, CD33 double positive targets.

FIG. 9: Expression of Target Antigens on Artificial Target Cells

A. Shows flow cytometry scatter plots CD19 vs CD33 of the original set of artificial target cells derived from SupT1 cells. From left to right: double negative SupT1 cells, SupT1 cells positive for CD19, positive for CD33 and positive for both CD19 and CD33. B. Shows flow cytometry scatter plots CD19 vs GD2 of the artificial target cells generated to test the CD19 AND GD2 gate: From left to right: negative SupT1 cells, SupT1 cells expressing CD19, SupT1 cells transduced with GD2 and GM3 synthase vectors which become GD2 positive and SupT1 cells transduced with CD19 as well as GD2 and GM3 synthase which are positive for both GD2 and CD19. C. Shows flow cytometry scatter plots of CD19 vs EGFRvIII of the artificial targets generated to test the CD19 AND EGFRvIII gate. From left to right: negative SupT1 cells, SupT1 cells expressing CD19, SupT1 cells transduced with EGFRvIII and SupT1 cells transduced with both CD19 and EGFRvIII. D. Shows flow cytometry scatter plots of CD19 vs CD5 of the artificial targets generated to test the CD19 AND CD5 gate. From left to right: negative 293T cells, 293T cells transduced with CD19, 293T cells transduced with CD5, 293T cells transduced with both CD5 and CD19 vectors.

FIG. 10: Generalizability of the AND Gate

A. Cartoon of AND gate modified so the second CAR's specificity is changed from the original specificity of CD33, to generate 3 new CARs: CD19 AND GD2, CD19 AND EGFRvIII, CD19 AND CD5. B. CD19 AND GD2 AND gate: Left: expression of AND gate is shown recombinant CD19-Fc staining (x-axis) for the CD19 CAR, versus anti-human-Fc staining (Y-axis) for the GD2 CAR. Right: function in response to single positive and double positive targets. C. CD19 AND EGFRvIII AND gate: Left: expression of AND gate is shown recombinant CD19-Fc staining (x-axis) for the CD19 CAR, versus anti-human-Fc staining (Y-axis) for the EGFRvIII CAR. Right: function in response to single positive and double positive targets. D. CD19 AND CD5 AND gate: Left: expression of AND gate is shown recombinant CD19-Fc staining (x-axis) for the CD19 CAR, versus anti-human-Fc staining (Y-axis) for the CD5 CAR. Right: function in response to single positive and double positive targets.

FIG. 11: Kinetic Segregation Model of CAR Logic Gates

Model of kinetic segregation and behaviour of AND gate, NOT AND gate and controls. CARs recognize either CD19 or CD33. The immunological synapse can be imagined between the blue line, which represents the target cell membrane and the red line, which represents the T-cell membrane. ‘45’ is the native CD45 protein present on T-cells. ‘H8’ is a CAR ectodomain with human CD8 stalk as the spacer. ‘Fc’ is a CAR ectodomain with human HCH2CH3 as the spacer. ‘M8’ is a CAR ectodomain with murine CD8 stalk as the spacer. ‘19’ represents CD19 on the target cell surface. ‘33’ represents CD33 on the target cell surface. The symbol ‘⊕’ represents an activating endodomain containing ITAMS. The symbol ‘⊖’ represents a phosphatase with slow kinetics—a ‘ligation on’ endodomain such as one comprising of the catalytic domain of PTPN6 or an ITIM. The symbol ‘Ø’ represents a phosphatase with fast kinetics—a ‘ligation off’ endodomain such as the endodomain of CD45 or CD148. This symbol is enlarged in the figure to emphasize its potent activity.

(a) Shows the postulated behaviour of the functional AND gate which comprises of a pair of CARs whereby the first CAR recognizes CD19, has a human CD8 stalk spacer and an activating endodomain; and the second CAR recognizes CD33, has an Fc spacer and a CD148 endodomain;
(b) Shows the postulated behaviour of the control AND gate. Here, the first CAR recognizes CD19, has a human CD8 stalk spacer and an activating endodomain; and the second CAR recognizes CD33, but has a mouse CD8 stalk spacer and a CD148 endodomain;
(c) Shows the behaviour of a functional AND NOT gate which comprises of a pair of CARs whereby the first CAR recognizes CD19, has a human CD8 stalk spacer and an activating endodomain; and the second CAR recognizes CD33, has a mouse CD8 stalk spacer and a PTPN6 endodomain;
(d) Shows the postulated behaviour of the control AND NOT gate which comprises of a pair of CARs whereby the first CAR recognizes CD19, has a human CD8 stalk spacer and an activating endodomain; and the second CAR recognizes CD33, but has an Fc spacer and a PTPN6 endodomain;

In the first column, target cells are both CD19 and CD33 negative. In the second column, targets are CD19 negative and CD33 positive. In the third column, target cells are CD19 positive and CD33 negative. In the fourth column, target cells are positive for both CD19 and CD33.

FIG. 12: AND Gate Functionality in Primary Cells

PBMCs were isolated from blood and stimulated using PHA and IL-2. Two days later the cells were transduced on retronectin coated plates with retro virus containing the CD19:CD33 AND gate construct with the Tyrp1 retention signal placed either distal or proximal to the endodomain (see legend for FIG. 17). On day 5 the expression level of the two CARs translated by the AND gate construct was evaluated via flow cytometry and the cells were depleted of CD56+ cells (predominantly NK cells). On day 6 the PBMCs were placed in a co-culture with target cells at a 1:2 effector to target cell ratio. On day 8 the supernatant was collected and analysed for IFN-gamma secretion via ELISA. The results are aggregated from four different donors.

FIG. 13: A Selection/Hierarchy of Possible Spacer Domains of Increasing Size is Shown.

The ectodomain of CD3-Zeta is suggested as the shortest possible spacer, followed by the (b) the IgG1 hinge. (c) murine or human CD8 stalk and the CD28 ectodomains are considered intermediate in size and co-segregate. (d) The hinge, CH2 and CH3 domain of IgG1 is bigger and bulkier, and (e) the hinge, CH2, CH3 and CH4 domain of IgM is bigger still. Given the properties of the target molecules, and the epitope of the binding domains on said target molecules, it is possible to use this hierarchy of spacers to create a CAR signaling system which either co-segregates or segregates apart upon synapse formation.

FIG. 14: Matrix for the AND Gate Platform.

Multiple AND gates were produced and tested with various combinations of the spacers: hinge; huCD8STK; huCD28ecto; hulgG (HCH2CH3pvaa); and hulgM (CH2HCH3CH4) as described in the legend for FIG. 13. They were tested in the AND gate platform as previously described with a CD19 signalling CAR and a CD33 inhibitory CAR with a CD148 ligation-off inhibitory endodomain. Blue=predicted; Red=proven; NO STIM=no stimulation with any cognate targets; AND=functions as AND gate.

FIG. 15: Methods Utilised to Express Different Proteins from the Same Vector

(a) Two different promoters within the same cassette result in two different transcripts which each give rise to separate proteins. (b) Use of an Internal Ribosome Entry sequence (IRES) leads to a single transcript which is translated into two separate proteins. (c) Use of the FMDV 2A peptide results in a single transcript, and a single polyprotein which rapidly cleaves into two separate proteins.

FIG. 16: TYRP1 Endodomain is Able to Direct the Retention of a Transmembrane Protein with a Complex Endodomain

Tyrp1 is a type I transmembrane protein, 537aa long. The di-leucine motif, which retains the protein in the intracellular compartment, is indicated as a black rectangle on the cytoplasmic domain. (A) Tyrp1 (wt). Wild type Tyrp1 consists of a peptide signal, a luminal domain, a transmembrane domain, and a cytoplasmic domain. The cytoplasmic domain contains the di-leucine retention signal. (B) Tyrp1 (wt)-SG Linker-eGFP. This construct contains the wild type Tyrp1 simply fused to eGFP via a serine-glycine-glycine-glycine-serine linker. The Tyrp1-L-eGFP represents the cytoplasmic-proximal Tyrp1. (C) Tyrp1 Lumenal (LM)-Transmembrane (TM)-SG Linker-eGFP-Tyrp1 Cytoplasmic (CP). This construct constitutes the cytoplasmic-distal Tyrp1, since SG linker-eGFP interposes between the transmembrane and cytoplasmic domains. D: Tyrp1 Lumenal (LM)-Transmembrane (TM)-SG Linker-eGFP. This construct serves as the positive control, as the cytoplasmic domain containing the retention signal has been excluded. All constructs are co-expressed with IRES.CD34. Staining of transduced SupT1 cells is shown with intracellular and surface staining bottom left/right respectively.

FIG. 17: Functionality of the TYRP1 Retention Signal in Primary Cells

A construct was generated which co-express an anti-CD19 and an anti-CD33 CAR using a FMD-2A like peptide. Two variants of this construct were also generated: in the first variant, the di-leucine motif from TYRP1 was inserted into the anti-CD19 CAR endodomain just proximal to the TM domain; In the second variant the same TYRP1 di-lecuine motif was attached to the carboxy-terminus of the anti-CD19 CAR endodomain. PBMCs were isolated from blood and stimulated using PHA and IL-2. Two days later the cells were transduced on retronectin coated plates with retro virus containing the different CD19:CD33 CAR constructs. On day 5 the expression level of the two CARs translated by the construct was evaluated via flow cytometry using recombinant CD19-Fc and CD33-Fc fusions. A. Shows cartoon of the synthetic gene constructed to allow co-expression; B. Shows a cartoon of the subsequent pairs of proteins generated by the three constructs; C. Shows expression of the two receptors by flow-cytometry. In the original construct, both CARs are equally expressed. With incorporation of the di-leucine motif distally in the endodomain of the anti-CD19 CAR, the CD33 CAR expression remains constant but the CD19 expression drops to intermediate levels. With incorporation of the di-leucine motif proximally in the endodomain of the anti-CD19 CAR, the CD33 CAR expression remains constant, but the CD19 expression drops to low levels.

FIG. 18: Retention Signal from Cytosolic Tail of E3/19K

A construct was generated which co-expresses an anti-CD19 and an anti-CD33 CAR using a FMD-2A like peptide. Two variants of this construct were also generated: in the first variant, the last 6aa from E3/19K (DEKKMP), which were found to be critical for its Golgi/ER retention ability, were attached to the carboxy-terminus of the anti-CD33 CAR endodomain; in the second variant, the entire cytosolic tail of adenovirus E3/19K protein was attached to the carboxy-terminus of the anti-CD33 CAR endodomain

FIG. 19: Functionality of E3/19K Retention Signal

The constructs shown in FIG. 4 were transfected into 293T cells and the expression level of the two CARs translated by the construct was evaluated via flow cytometry using recombinant CD19-Fc and CD33-Fc fusions. A clear retention was observed when the full length adenovirus E3/19K protein, or the DEKKMP motif was placed on the anti-CD33 receptor. The anti-CD19 receptor expression levels were unaffected.

FIG. 20: Schematic diagram illustrating the function of signal sequences in protein targeting

FIG. 21: Schematic diagram of nucleic acid construct encoding two CARs

FIG. 22: Verifying the Function of a Substituted Signal Sequence.

PCT/GB2014/053452 describes vector system encoding two chimeric antigen receptors (CARs), one against CD19 and one against CD33. The signal peptide used for the CARs in that study was the signal peptide from the human CD8a signal sequence. For the purposes of this study, this was substituted with the signal peptide from the murine Ig kappa chain V-III region, which has the sequence: METDTLILWVLLLLVPGSTG (hydrophobic residues highlighted in bold). In order to establish that the murine Ig kappa chain V-III signal sequence functioned as well as the signal sequence from human CD8a, a comparative study was performed. For both signal sequences, functional expression of the anti-CD33 CAR and the anti-CD19 CAR was observed.

FIG. 23: Testing the effect of one amino acid deletion in the murine Ig kappa chain V-Ill. Mutant 1 kappa chain was created with the following deletion (shown in grey) in the h-region METDT [symbol] ILWVLLLLVPGSTG and the relative expression on the anti-CD33 CAR and the anti-CD19 CAR was observed.

FIG. 24: Testing the effect of two amino acid deletions in the murine Ig kappa chain V-Ill. Mutant 2 kappa chain was created with the following deletions (shown in grey) in the h-region METDT [symbol] ILWVLLL [symbol] VPGSTG and the relative expression on the anti-CD33 CAR and the anti-CD19 CAR was observed.

FIG. 25: Testing the effect of three amino acid deletions in the murine Ig kappa chain V-Ill. Mutant 2 kappa chain was created with the following deletions (shown in grey) in the h-region METDT [symbol] ILWVLLL [symbol] PGSTG and the relative expression on the anti-CD33 CAR and the anti-CD19 CAR was observed.

FIG. 26: Testing the effect of five amino acid deletions in the murine Ig kappa chain V-Ill. Mutant 2 kappa chain was created with the following deletions (shown in grey) in the h-region METDT [symbol] L [symbol] VLLL [symbol] PGSTG and the relative expression on the anti-CD33 CAR and the anti-CD19 CAR was observed.

### SUMMARY OF ASPECTS OF THE INVENTION

The present inventors have developed a panel of “logic-gated” chimeric antigen receptor pairs which, when expressed by a cell, such as a T cell, are capable of detecting a particular pattern of expression of at least two target antigens. If the at least two target antigens are arbitrarily denoted as antigen A and antigen B, the three possible options are as follows:

“OR GATE”—T cell triggers when either antigen A or antigen B is present on the target cell “AND GATE”—T cell triggers only when both antigens A and B are present on the target cell “AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

The present inventors have also found that, when a CAR is co-expressed with a second CAR as a polyprotein which after translation is subsequently cleaved to separate the two CARs, it is possible to modulate the relative cell surface expression of the two CARs by reducing trafficking to the cell surface of one or both CAR(s) and/or by reducing its half-life at the cell surface. This need not be limited to a pair of CARs, but may be used to allow control of the relative expression of multiple proteins initially translated as a polyprotein.

As used herein, ‘polyprotein’ refers to a polypeptide sequence translated from a single nucleic acid construct as a single entity, but which comprises polypeptide sequences which are subsequently separated and which function as discrete entities (e.g. separate CARs).

The present invention relates to an AND logic gate, in which the relative expression of the two CARs is modulated.

Thus in a first aspect, the present invention provides a nucleic acid construct comprising the following structure:

A-X-B
in which
X is a nucleic acid sequence which encodes a cleavage site; and
A and B are nucleic acid sequences encoding a first and a second chimeric antigen receptor (CAR), each CAR comprising:
   (i) an antigen-binding domain;
   (ii) a spacer
   (iii) a trans-membrane domain; and
   (iv) an endodomain
wherein the antigen binding domains of the first and second CARs bind to different antigens, wherein the spacer of the first CAR is different to the spacer of the second CAR and wherein one of the first or second CARs is an activating CAR comprising an activating endodomain and the other CAR is an inhibitory CAR comprising a ligation-off inhibitory endodomain;
and wherein:
   (a) the first and/or second CAR comprises an intracellular retention signal; and/or
   (b) the signal peptide of the first or second CAR comprises one or more mutation(s) such that it has fewer hydrophobic amino acids.

For (b) the mutated signal peptide may have fewer hydrophobic amino acids than the “wild-type” signal peptide sequence from which it is derived. The mutated signal peptide may have fewer hydrophobic amino acids than the signal peptide of the other CAR.

The spacer of the first CAR may have a different length and/or charge and/or shape and/or configuration and/or glycosylation to the spacer of the second CAR, such that when the first CAR and the second CAR bind their respective target antigens, the first CAR and second CAR become spatially separated on the T cell. Ligation of the first and second CARs to their respective antigens causes them to be compartmentalized together or separately in the immunological synapse resulting in control of activation. This may be understood when one considers the kinetic separation model of T-cell activation (see below).

The first spacer or the second spacer may comprise a CD8 stalk and the other spacer may comprise the hinge, CH2 and CH3 domain of an IgG1.

In the present invention, which relates to the “AND” gate, one of the first or second CARs is an activating CAR comprising an activating endodomain, and the other CAR is a “ligation-off” inhibitory CAR comprising an inhibitory endodomain. The ligation-off inhibitory CAR inhibits T-cell activation by the activating CAR in the absence of inhibitory CAR ligation, but does not significantly inhibit T-cell activation by the activating CAR when the inhibitory CAR is ligated. Since the spacer of the first CAR has a different length and/or charge and/or shape and/or configuration and/or glycosylation from the spacer of the second CAR, when both CARs are ligated they segregate. This causes the inhibitory CAR to be spatially separated from the activating CAR, so that T cell activation can occur. T cell activation therefore only occurs in response to a target cell bearing both cognate antigens.

The inhibitory endodomain may comprise all or part of the endodomain from a receptor-like tyrosine phosphatase, such as CD148 or CD45.

The antigen-binding domain of the first CAR may bind CD5 and the antigen-binding domain of the second CAR may bind CD19. This is of use in targeting chronic lymphocytic leukaemia (CLL). This disease can be treated by targeting CD19 alone, but at the cost of depleting the entire B-cell compartment. CLL cells are unusual in that they co-express CD5 and CD19. Targeting this pair of antigens with an AND gate will increase specificity and reduce toxicity.

The present invention relates to a method for modulating the relative expression of two (or more) CARs in an AND logic gate, by one or both of the following approaches (a) by incorporation of an intracellular retention signal in one or both CAR(s) and (b) by altering the signal peptide of one CAR in order to remove or replace hydrophobic amino acids.

In particular, the relative expression level of the activating CAR, in relation to the inhibitory CAR, may be reduced by one of the above-mentioned approaches.

For approach (a), the endodomain of the CAR may comprise the intracellular retention signal.

The intracellular retention signal may direct the CAR away from the secretory pathway and/or to a membrane-bound intracellular compartment such as a lysozomal, endosomal or Golgi compartment.

The intracellular retention signal may, for example, be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

The intracellular retention signal may comprise a sequence selected from the group of: NPX′Y, YX′X′Z′, [DE]X′X′X′L[LI], DX′X′LL, DP[FW], FX′DX′F, NPF, LZX′Z[DE], LLDLL, PWDLW, KDEL, KKX′X′ or KX′KX′X′;

wherein X′ is any amino acid and Z′ is an amino acid with a bulky hydrophobic side chain.

The intracellular retention signal may comprise any of the sequences shown in Tables 1 to 5.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 1).

The intracellular retention signal may comprise the Adenoviral E3/19K intracellular retention signal. The intracellular retention signal may comprise the E3/19K cytosolic domain. The intracellular retention signal may comprise the sequence KYKSRRSFIDEKKMP (SEQ ID No. 2); or DEKKMP (SEQ ID No. 3).

The intracellular retention signal may be proximal or distal to a transmembrane domain of the CAR.

For approach (b), the signal peptides of the first and second CAR are different. They may differ in their number of hydrophobic amino acids. One signal peptide may comprise one or more mutation(s) such that it has fewer hydrophobic amino acids either a) than the wild-type sequence from which it was derived; or b) than the other signal peptide.

In particular the signal peptide of the activating CAR may be altered to remove or replace hydrophobic amino acids, such that the relative expression of the activating CAR, relative to the inhibitory CAR, is reduced at the cell surface.

The signal peptides may be different and the sequence of one signal peptide may be altered such that the hydrophobic amino acids are removed or replaced. Alternatively, the first signal peptide and the second signal peptide may be derivable from the same sequence, but one signal peptide may comprise one or more amino acid deletions/substitutions to remove/replace one or more hydrophobic amino acids compared to the other signal peptide.

The hydrophobic amino acid(s) removed or replaced may be selected from the group: Alanine (A); Valine (V); Isoleucine (I); Leucine (L); Methionine (M); Phenylalanine (P); Tyrosine (Y); Tryptophan (W) or the group Valine (V); Isoleucine (I); Leucine (L); and Tryptophan (W).

For approach (b) one signal peptide may comprise one, two, three, four or five mutations, such that it has one, two, three, four or five fewer hydrophobic amino acids than: the wild-type signal sequence from which it is derived and/or the other signal peptide.

In the nucleic acid construct of the present invention, the nucleic acid sequence of X may be a nucleic acid sequence encoding a self-cleaving peptide, a furin cleavage site or a Tobacco Etch Virus cleavage site.

The nucleic acid sequence of X may be a nucleic acid sequence encoding a 2A self-cleaving peptide from an aphtho- or a cardiovirus or a 2A-like peptide.

The nucleic acid construct may comprise a third nucleic acid sequence encoding a protein of interest (POI). The POI may be a transmembrane protein.

The POI may be selected from a list of: excitatory receptors such as 41BB, OX40, CD27, CD28 and related molecules; or inhibitory receptors such as PD1, CTLA4, LAIR1, CD22 and related molecules; or cytokine receptor molecules such as IL1R, IL2R, IL7R, IL15R and related molcules; or homing molecules such as N-CAM, V-CAM, L1-CAM, LFA-1, CDH1-3, Selectins or Integrins. The POI may be a third CAR.

The POI may be a synthetic protein such as a suicide gene or a marker gene.

The amount of a CAR which comprises an intracellular retention signal and/or which has an altered signal peptide which is expressed at the cell surface may be, for example, less than 90%, 70%, 50% or 30% compared to a CAR expressed from the same nucleic acid construct which does not comprise an intracellular retention signal and which has an unaltered signal peptide.

The nucleic acid construct may also encode a further polypeptide, for example a polypeptide which enables selection of transduced cells and/or enables cells expressing the polypeptide to be deleted.

Alternative codons may be used in regions of sequence encoding the same or similar amino acid sequences, in order to avoid homologous recombination.

In a second aspect the present invention provides a vector comprising a nucleic acid construct according to the first aspect of the invention.

The vector may be a retroviral vector or a lentiviral vector or a transposon.

In a third aspect the present invention provides a cell comprising a nucleic acid construct according to the first aspect of the invention or a vector according to the second aspect of the invention.

The cell may be an immune cell such as a T cell or a natural killer (NK) cell.

In a fourth aspect, the present invention provides a method for making a cell according to the third aspect of the invention, which comprises the step of introducing: a nucleic acid construct according to the first aspect of the invention or a vector according to the second aspect of the invention, into a cell.

The cell may be part of or derived from a sample isolated from a subject.

The cell used in the method of the fourth aspect of the invention may be from a sample isolated from a patient, a related or unrelated haematopoietic transplant donor, a completely unconnected donor, from cord blood, differentiated from an embryonic cell line, differentiated from an inducible progenitor cell line, or derived from a transformed cell line.

In a fifth aspect the present invention provides a pharmaceutical composition comprising a plurality of cells according to the forth aspect of the invention. The composition may be an autologous T and/or NK cell composition.

In a sixth aspect, the present invention provides a method for treating and/or preventing a disease, which comprises the step of administering a pharmaceutical composition according to the fifth aspect of the invention to a subject.

The method may comprise the following steps:
(i) isolation of a T and/or NK cell-containing sample from a subject;
(ii) transduction or transfection of the T and/or NK cells with: a nucleic acid construct according to the first aspect of the invention or a vector according to the second aspect of the invention; and
(iii) administering the T and/or NK cells from (ii) to a the subject.

There is also provided a pharmaceutical composition according to the fifth aspect of the invention for use in treating and/or preventing a disease.

There is also provided the use of a cell according to the third aspect of the invention in the manufacture of a medicament for treating and/or preventing a disease.

The invention further relates to method for modulating the relative cell surface expression of an activating CAR expressed from a single nucleic acid construct with a ligation-off inhibitory CAR by (a) including an intracellular retention signal in the nucleic acid sequence which encodes the activating CAR(s) and/or (b) altering the nucleic acid sequence which encodes the signal peptide of the activating CAR in order to remove or replace one or more hydrophobic amino acids in comparison with the signal peptide of the ligation-off inhibitory CAR. The nucleic acid construct may be as defined in the first aspect of the invention.

The kinetic-segregation based AND gate of the present invention offers a significant technical advantage to the previously described “co-CAR”, i.e. the dual targeting approach in which two antigens are recognized by two CARs which supply either an activating or a co-stimulating signal to the T-cell.

With the co-CAR approach, although greatest activity might be expected against target cells bearing both antigens, considerable activity against tissues bearing only antigen recognized by the activating CAR can be expected. This activity can be expected to be at least that of a first-generation CAR. First generation CARs have resulted in considerable toxicity: for instance biliary toxicity was observed in clinical testing of a first generation CAR recognizing Carbonic anhydrase IX which was unexpectedly expressed on biliary epithelium (Rotterdam ref). Notably, terminally differentiated effectors do not require or respond to co-stimulatory signals, so any terminally differentiated CAR T-cells would act maximally despite the absence of a co-stimulatory CAR signal.

Further, co-stimulatory signals lead to long-lasting effects on the T-cell population. These effects long outlast the T-cell/target synapse interaction. Consequently, CAR T-cells which become fully activated within the tumour and migrate could have maximally potent activity against single-antigen bearing normal tissues. This “spill-over” effect may be most pronounced in tissues within, near or which drain from the tumour. In fact, strategies based on the concept of the activity of a first generation CAR being enhanced by co-stimulatory signals engaged not CAR activation but through a distinct receptor, have been proposed and tested (Rossig, Blood. 2002 Mar. 15; 99(6):2009-16.).

The co-CAR approach hence can be expected to result at best to a reduction but not abolition of toxicity towards single antigen expressing normal tissue. The present invention uses kinetic segregation at the immunological synapse formed between the T-cell/target cell to regulate T-cell triggering itself. Consequently tight absolute control of triggering in the absence of the second antigen is achieved. Hence the totality of T-cell activation is restricted to target cells expressing both antigens, the AND gate should function irrespective of the effector cell type or differentiation state, and no “spill-over” effect AND gate T-cell activation is possible.

Moreover the capacity to modulate the relative expression of the two CARs, provided by the present invention, brings further advantages to the AND gate. When an AND gate system is working correctly, the ligation-off inhibitory CAR inhibits T-cell activation by the activating CAR in the absence of inhibitory CAR ligation, but does not significantly inhibit T-cell activation by the activating CAR when the inhibitory CAR is ligated. However, in some systems the activating CAR is “overactive” leading to a high level of background i.e. activation by the activating CAR in the absence of inhibitory CAR ligation. The present inventors have found that, by down regulating the relative expression of the activating CAR in comparison to the inhibitory CAR, it is possible to “tighten up” the system and reduce the level of background activation in the absence of the second antigen.

The inclusion of an intracellular retention signal in a CAR, or the alteration of the signal peptide of the CAR to reduce the number of hydrophobic amino acids, reduces the amount of the CAR expressed on the cell surface. As such, the relative expression level of two CARs expressed from a single construct can be modulated. As a CAR is only active at the cell surface, reducing the relative cell surface expression of the CAR also reduces its relative activity.

Thus the present invention provides a nucleic acid construct encoding an AND gate, in which the relative level of expression of the two or more CARs may be finely tuned, either to reduce the expression of the activating CAR (as described above) or to mirror the relative level of expression of the respective antigens on the target cell.

This invention can be extended to modulate the relative expression of three or more proteins expressed as a concatenated polypeptide, separated by cleavage sites and relative surface expression dictated by retention signals or signal peptides of differing activity.

### DETAILED DESCRIPTION

### Chimeric Antigen Receptors (Cars)

The present invention provides a nucleic acid construct which expresses two or more chimeric antigen receptors (CARs).

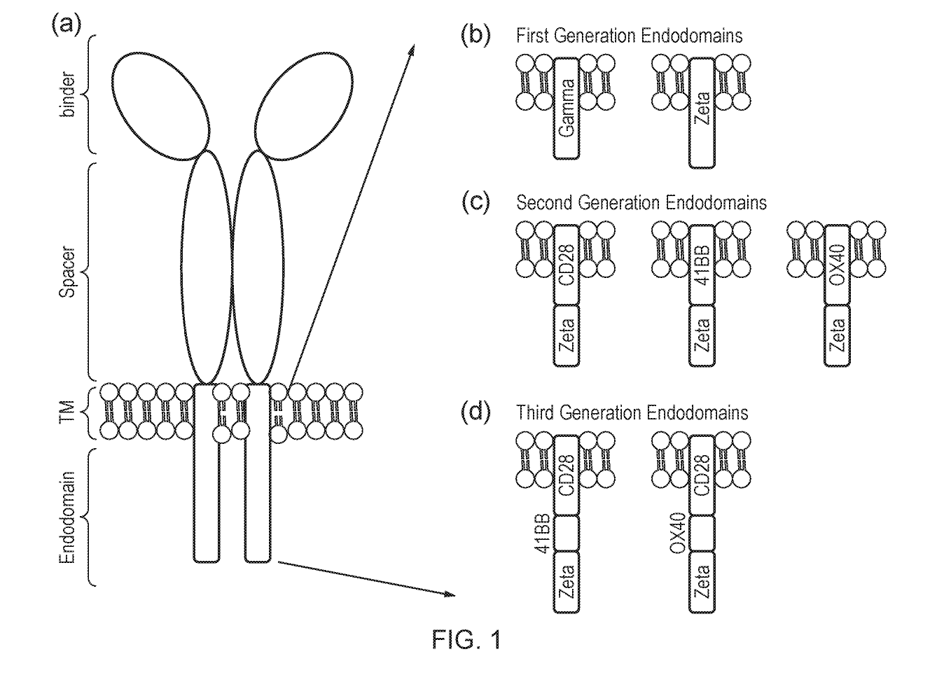

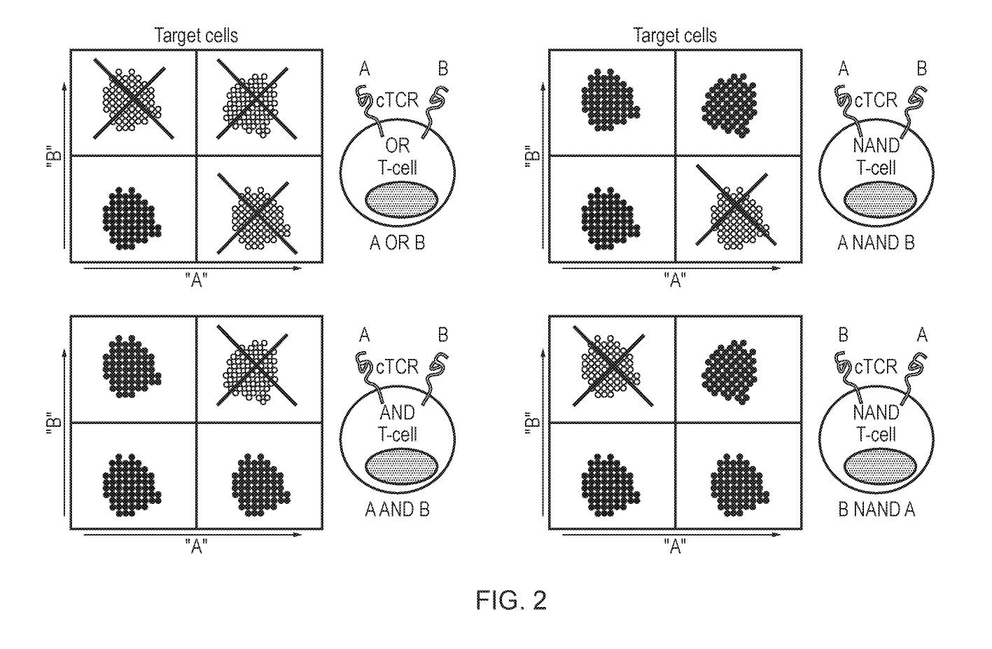

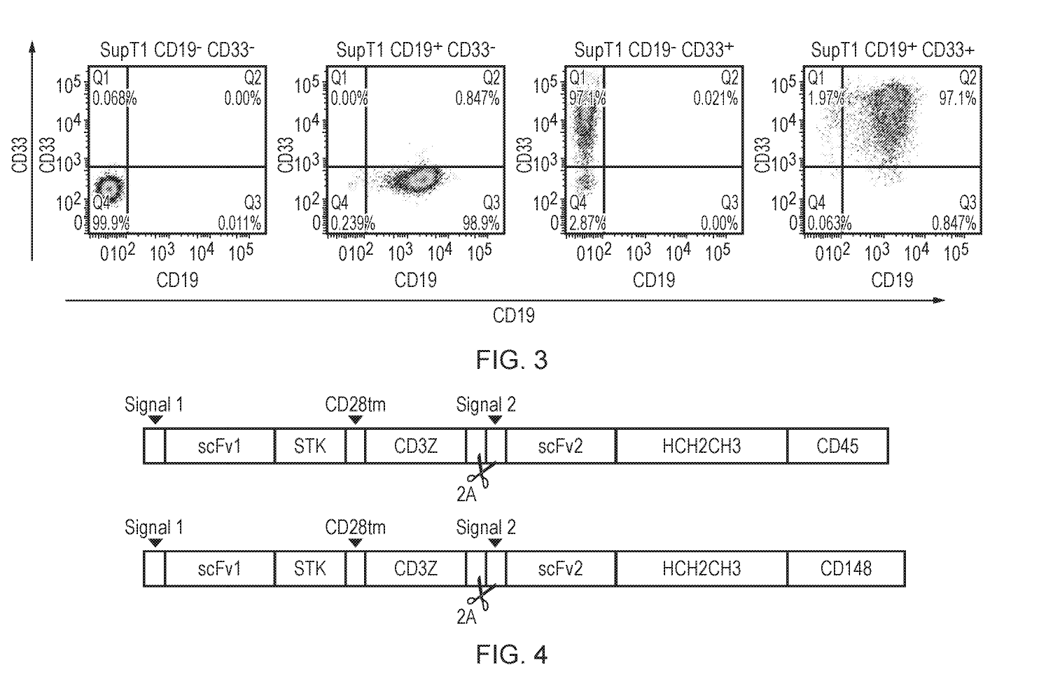

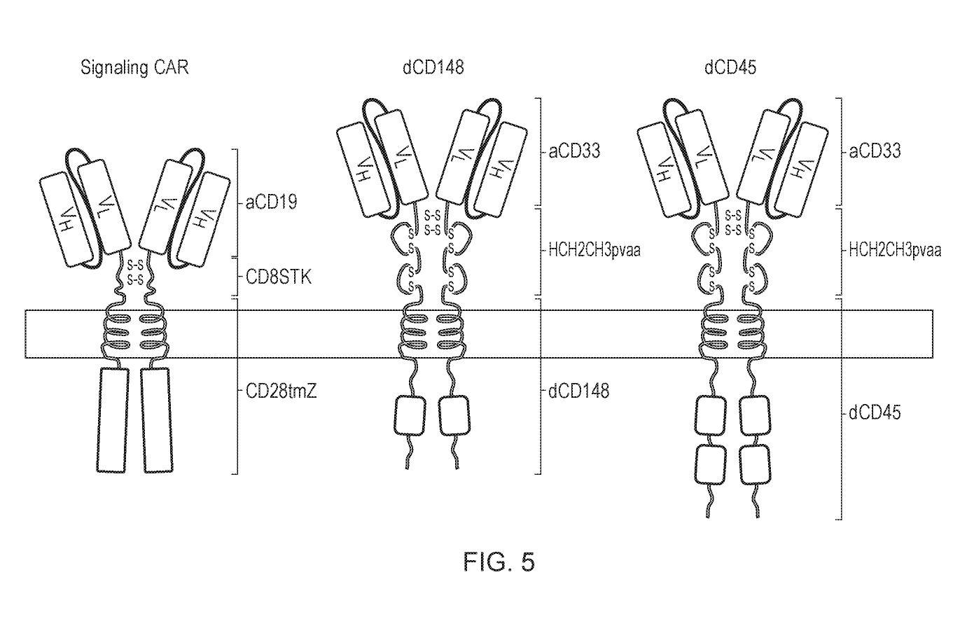

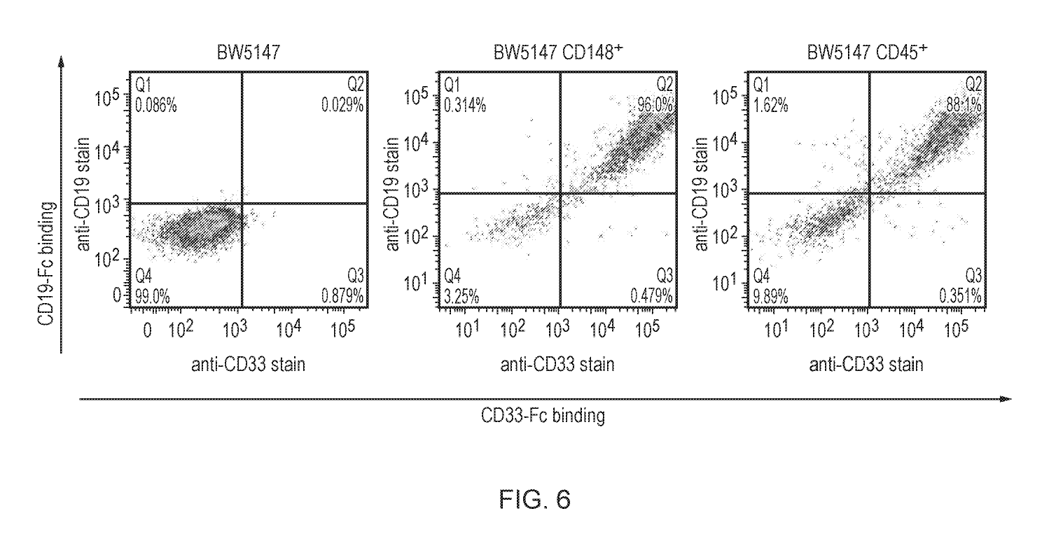

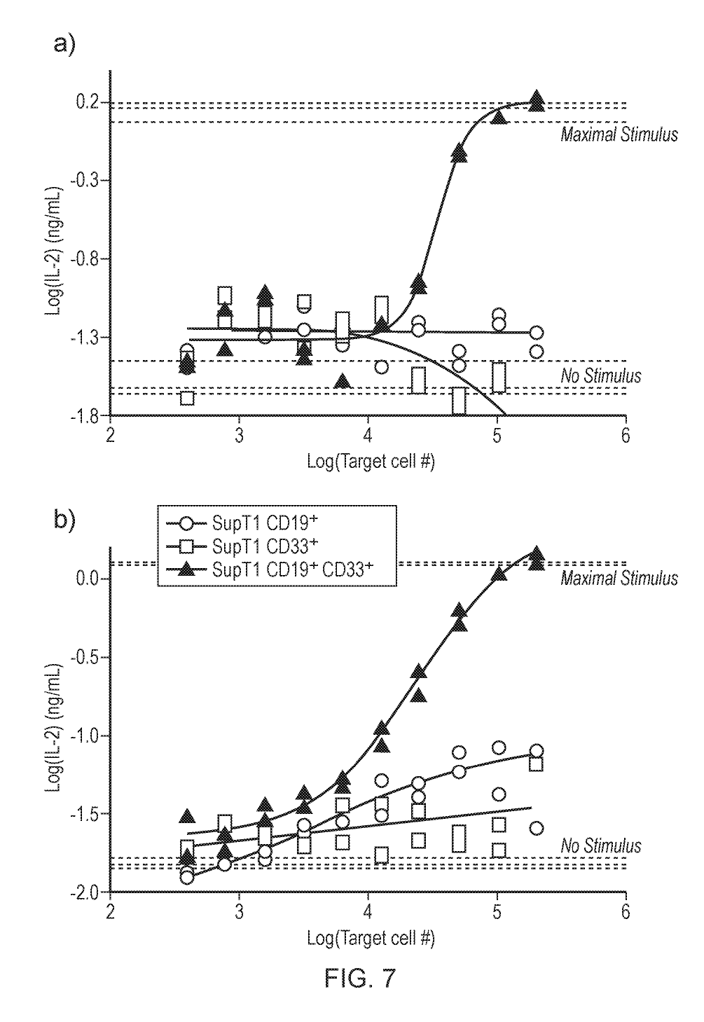

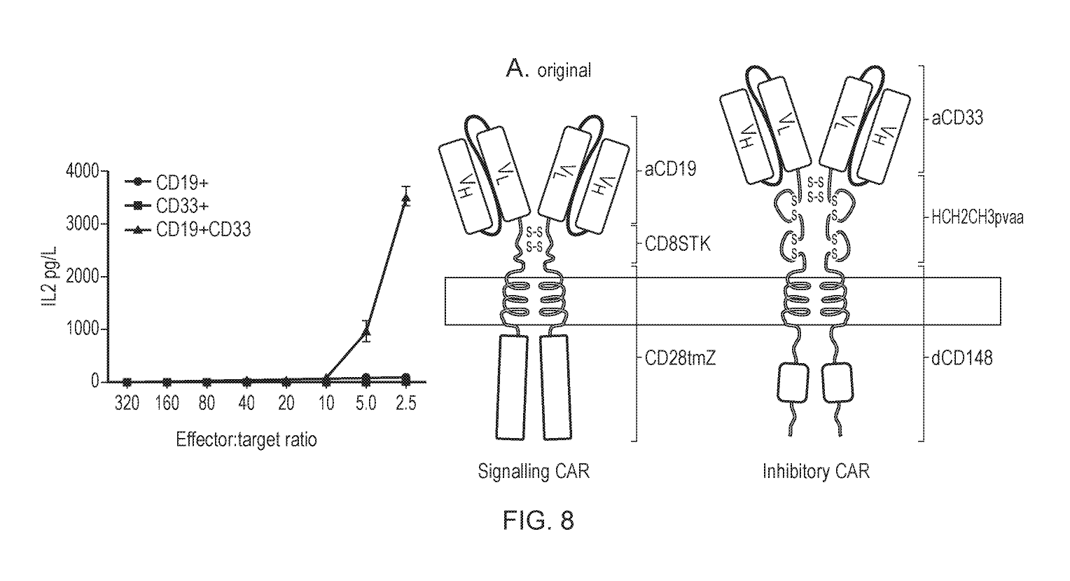

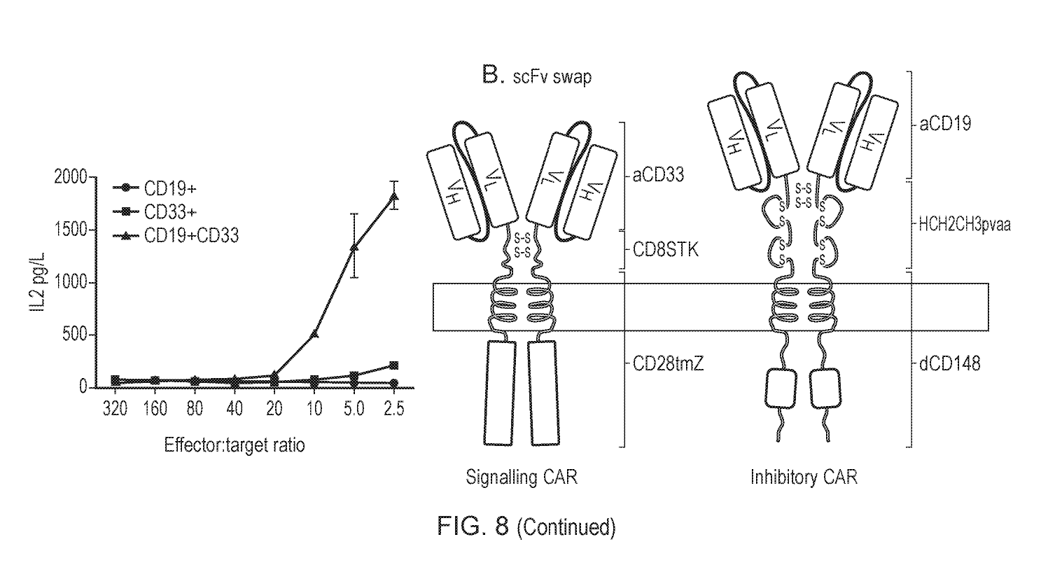

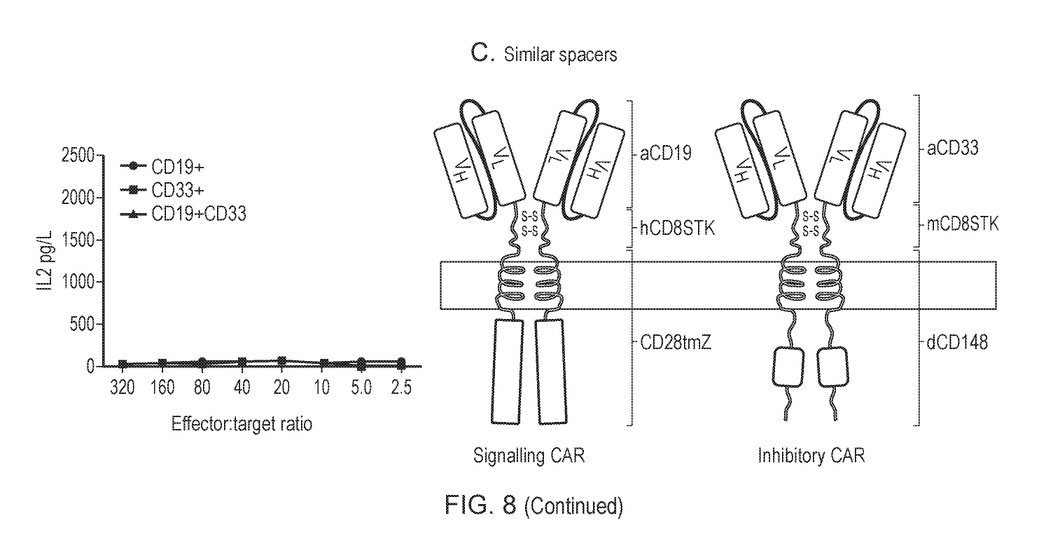

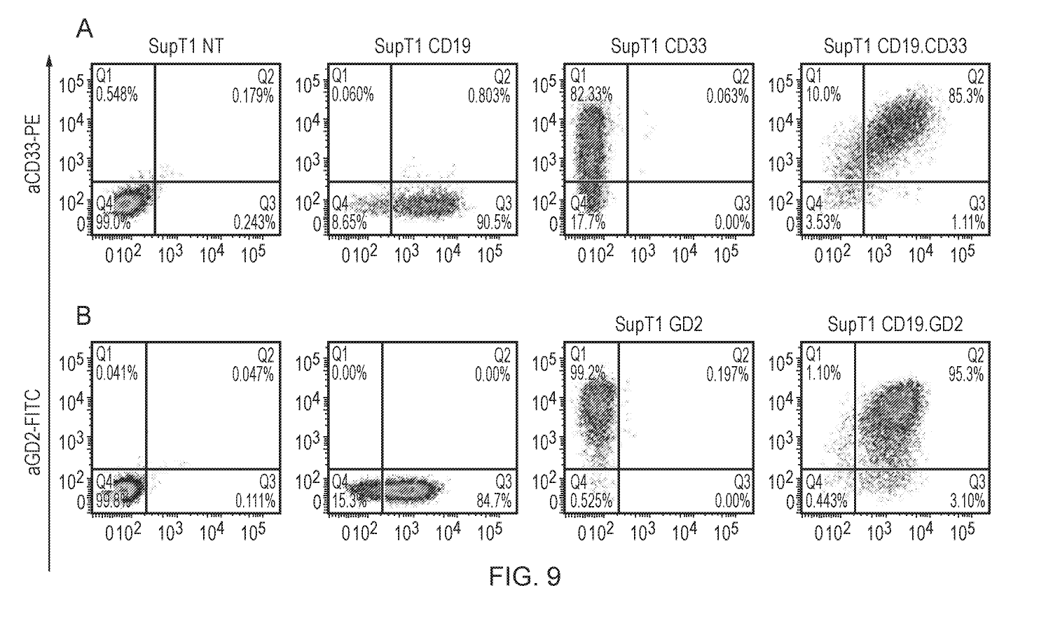

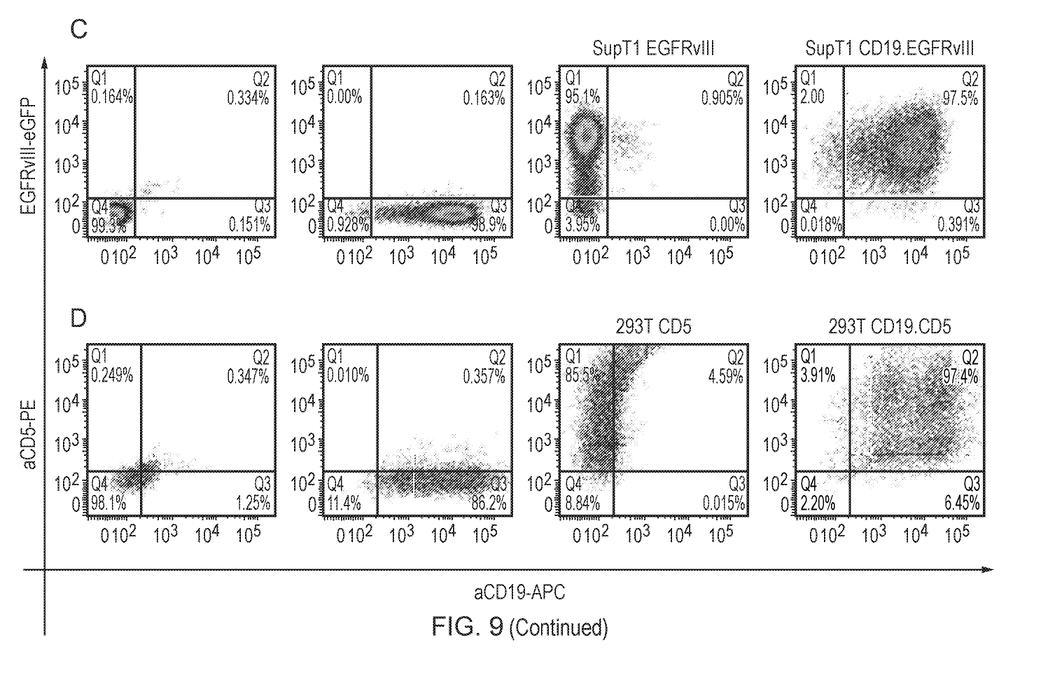

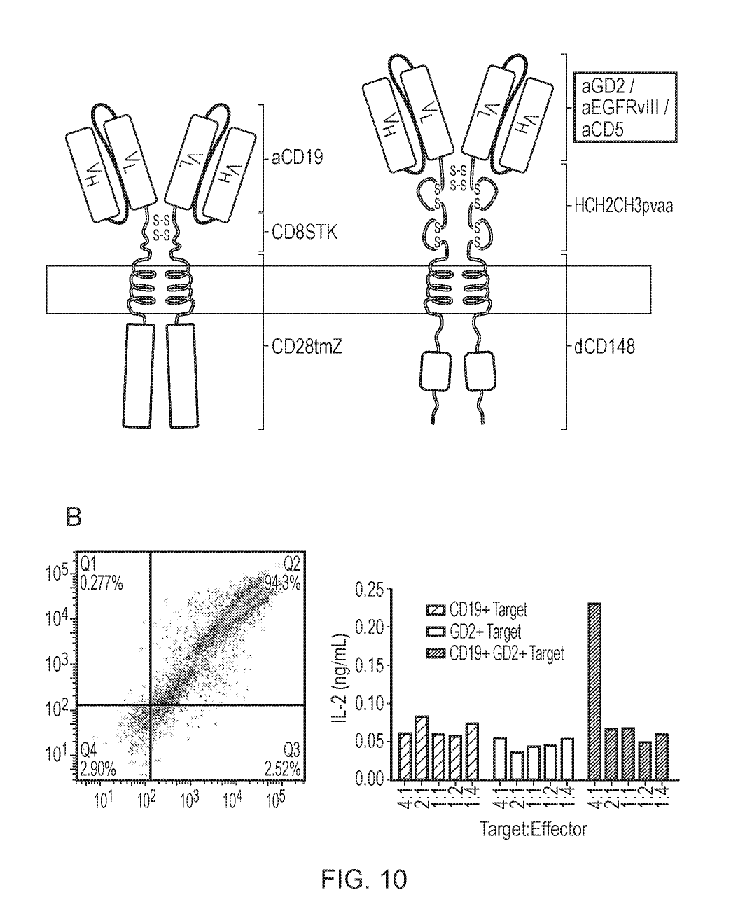

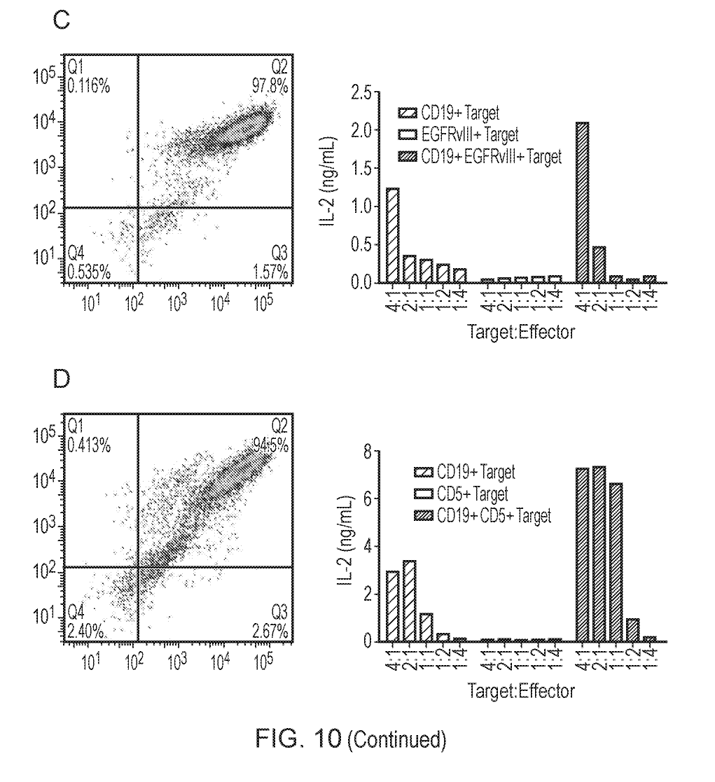

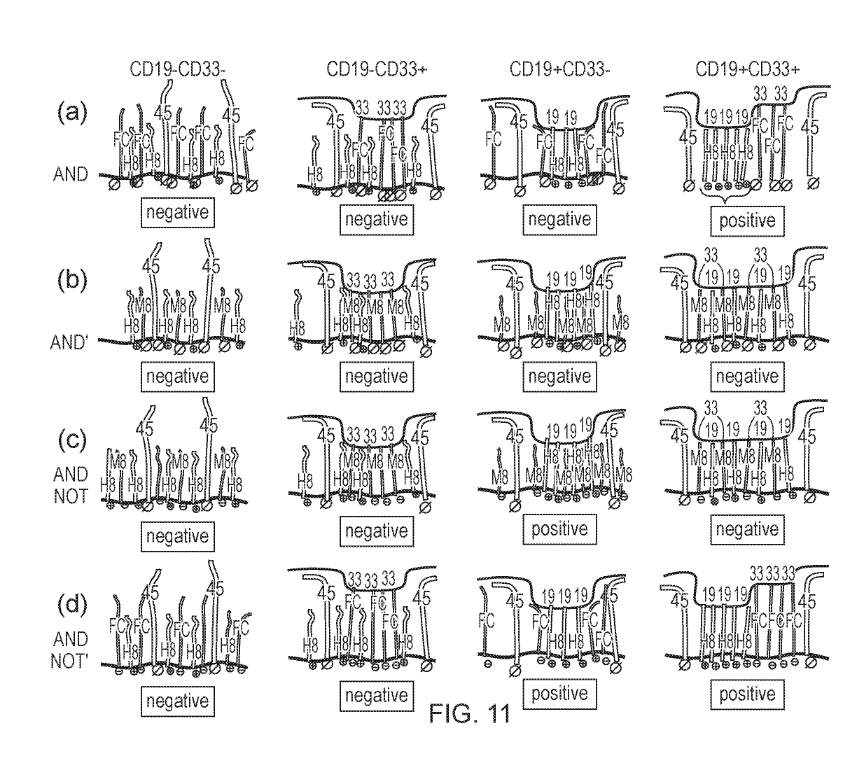

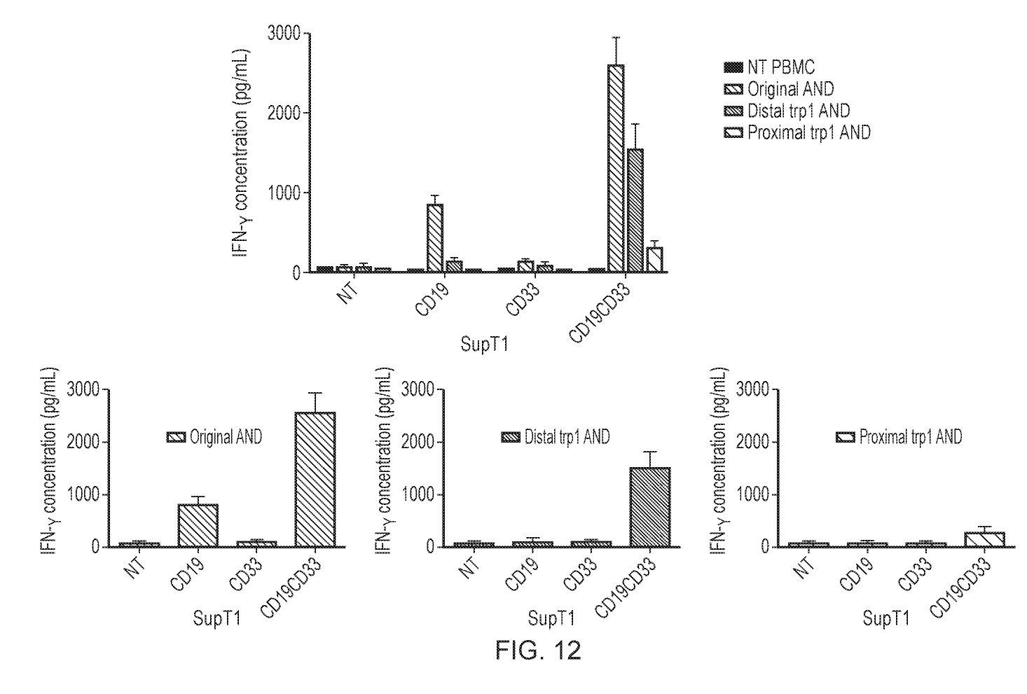

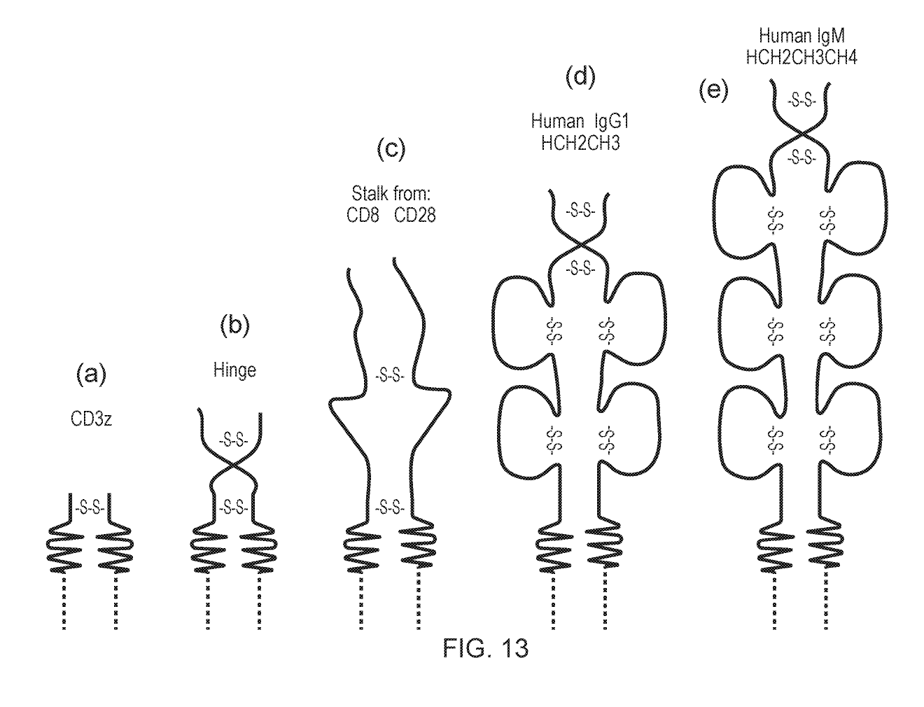

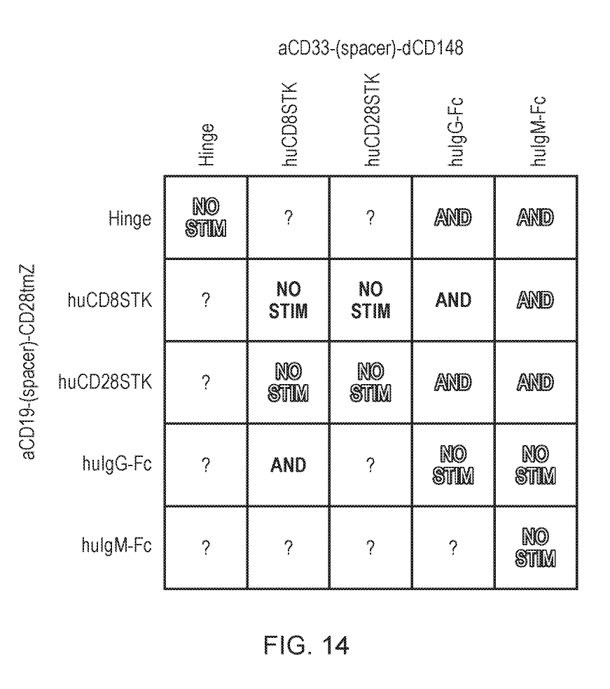

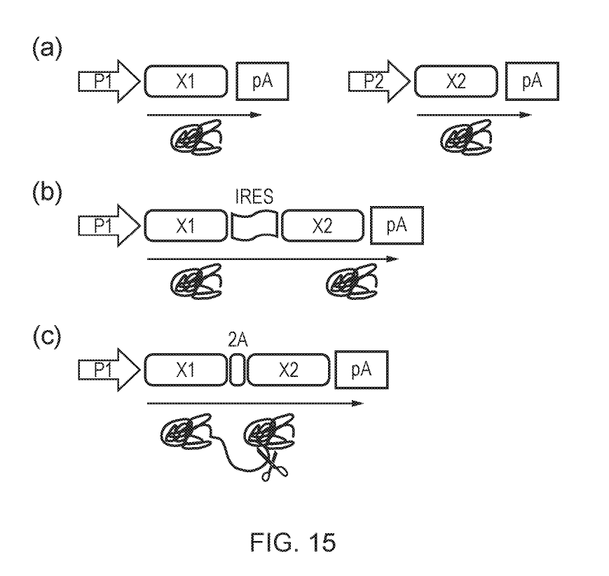

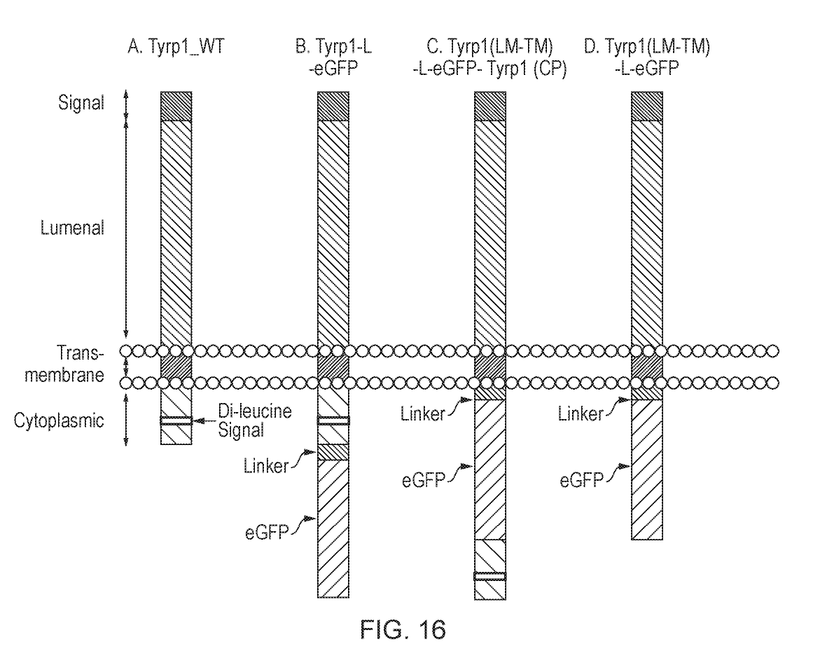

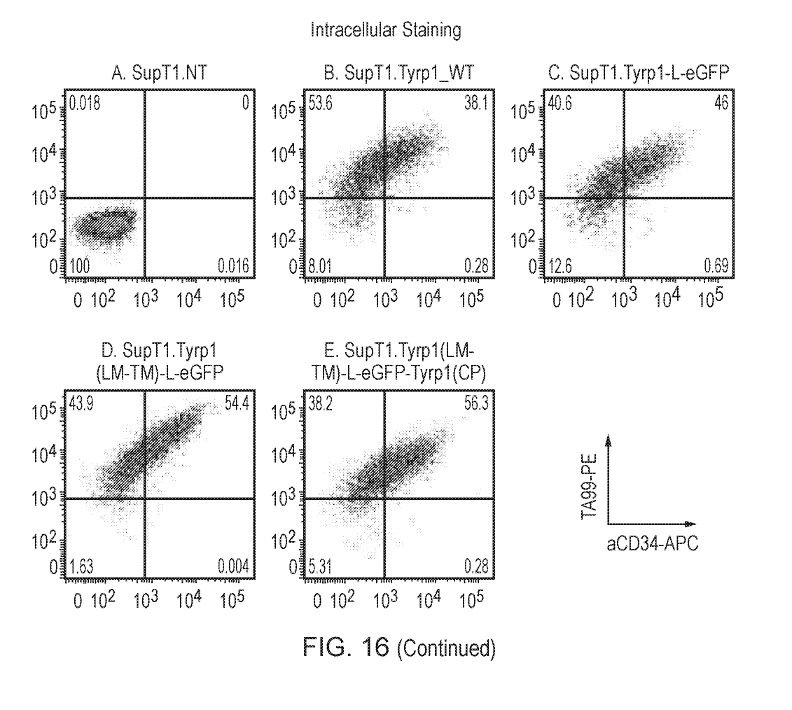

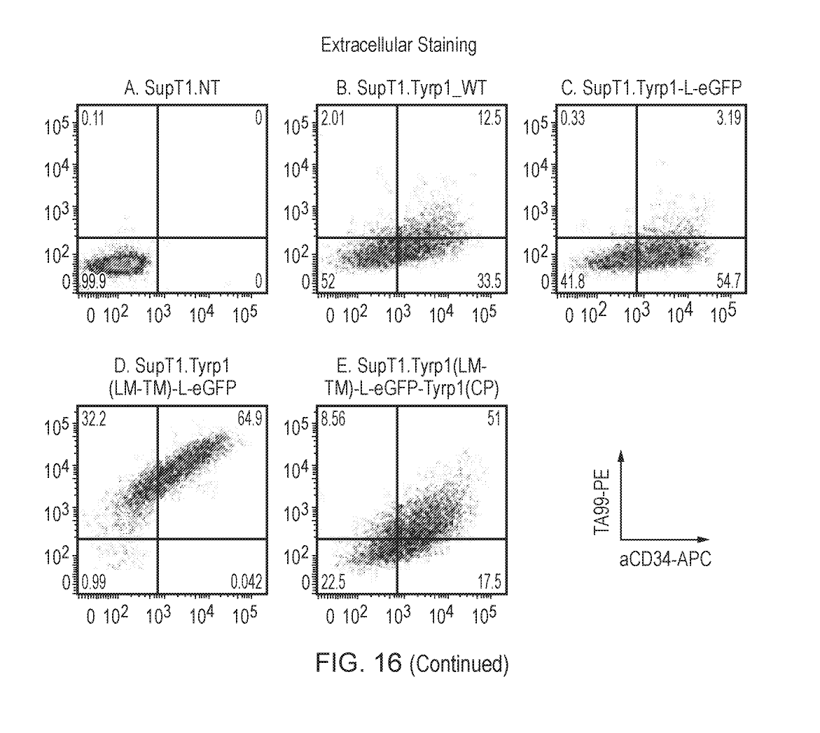

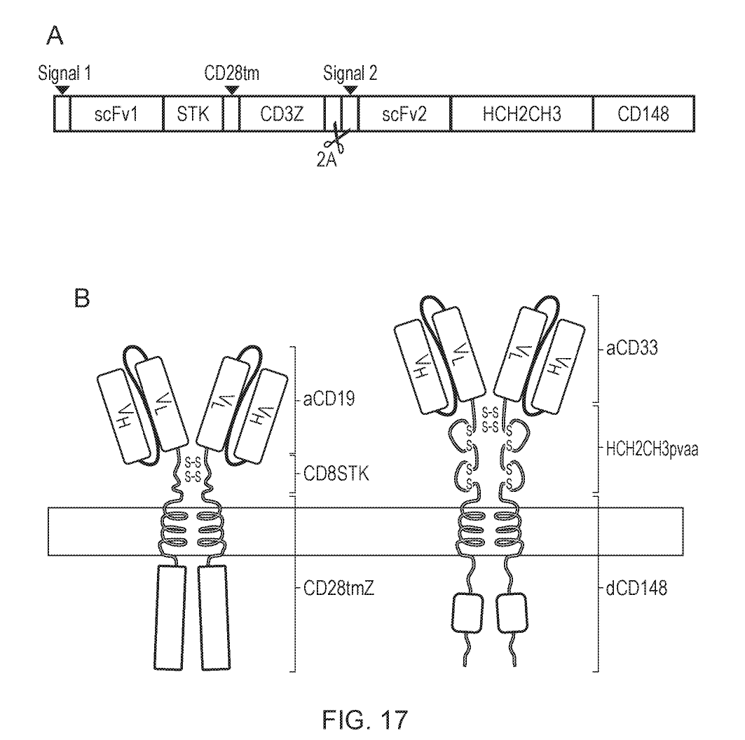

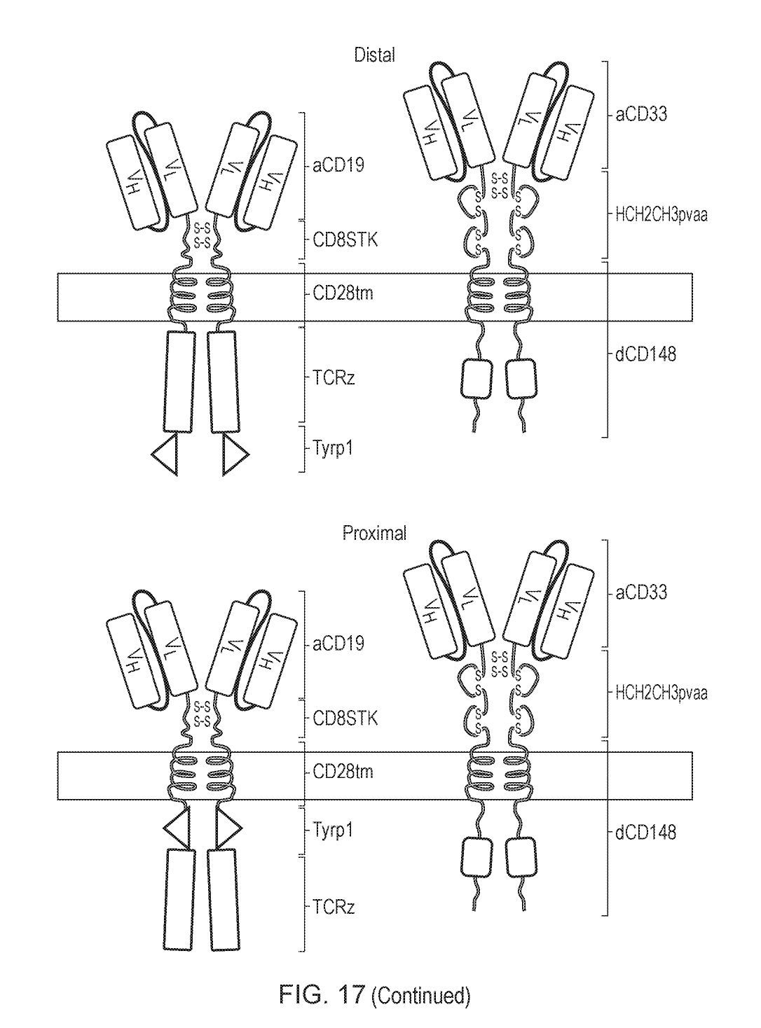

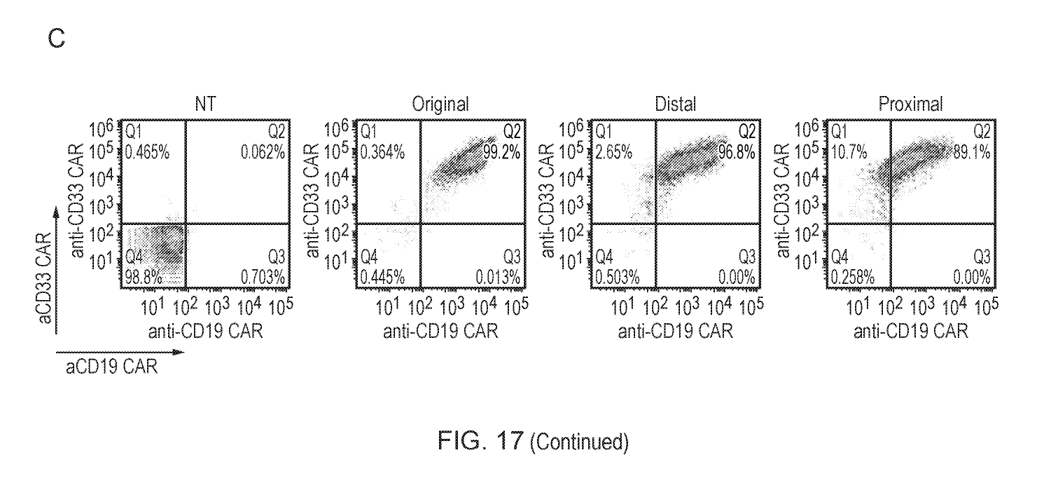

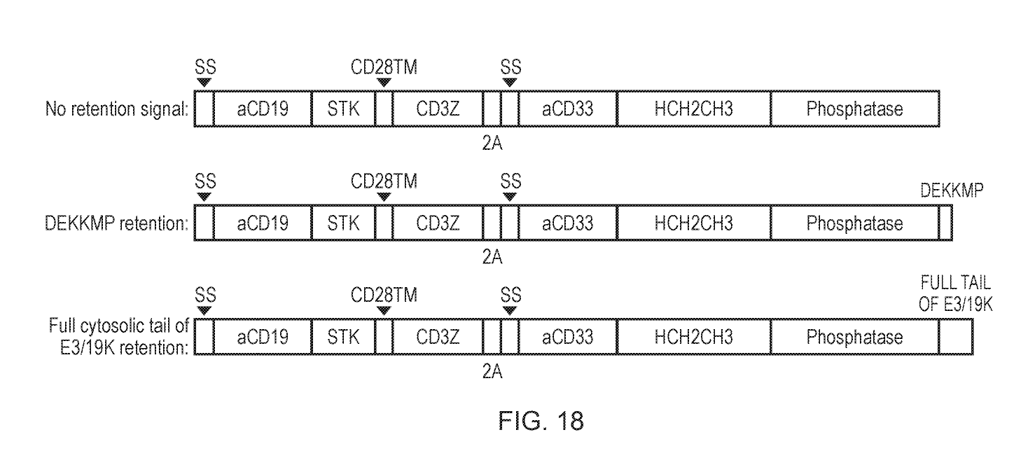

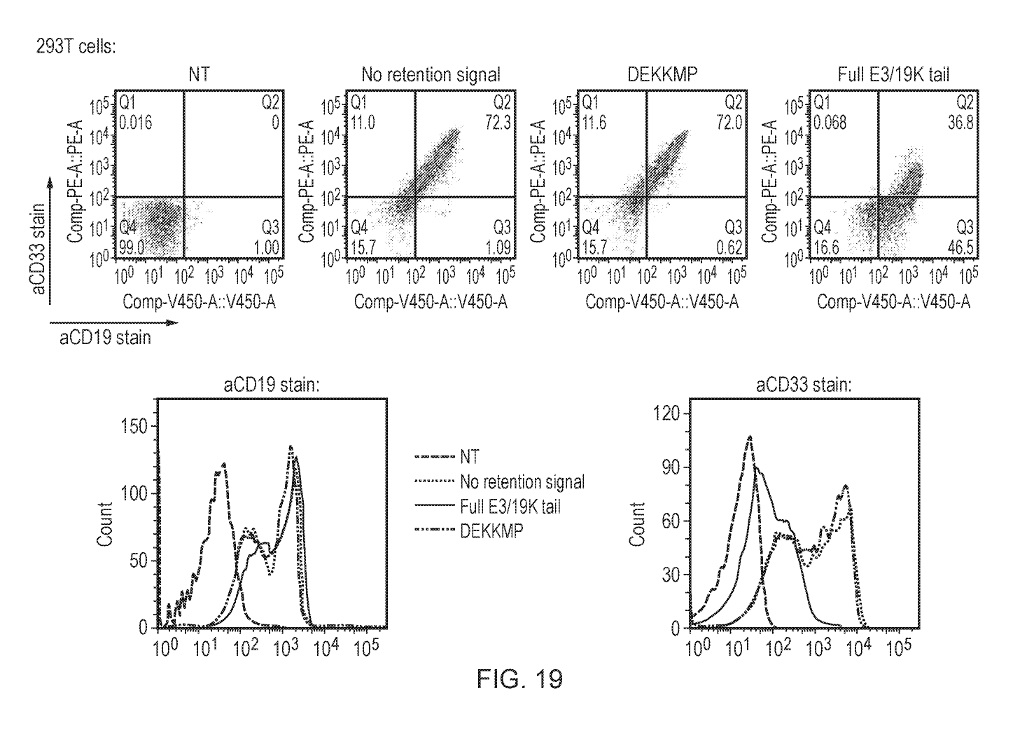

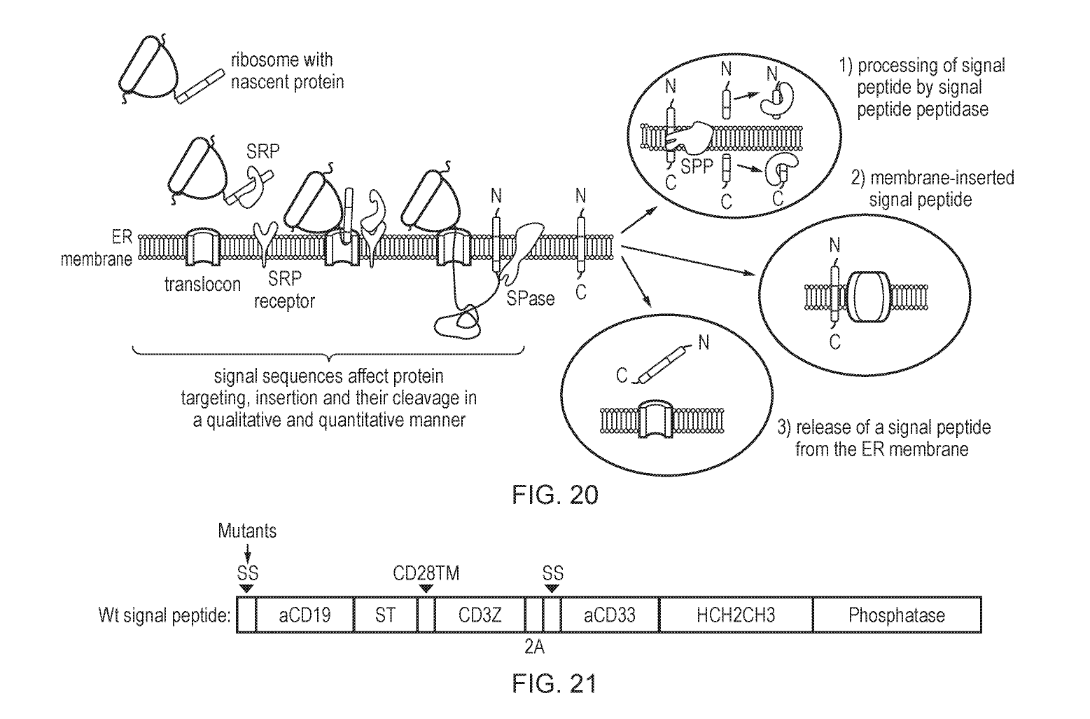

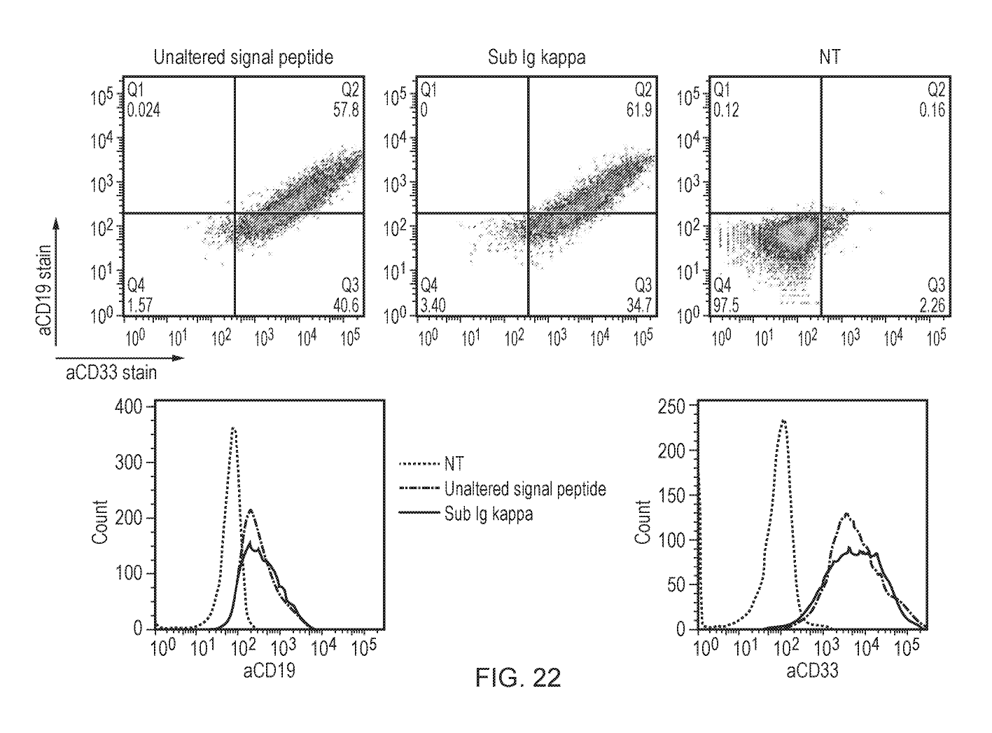

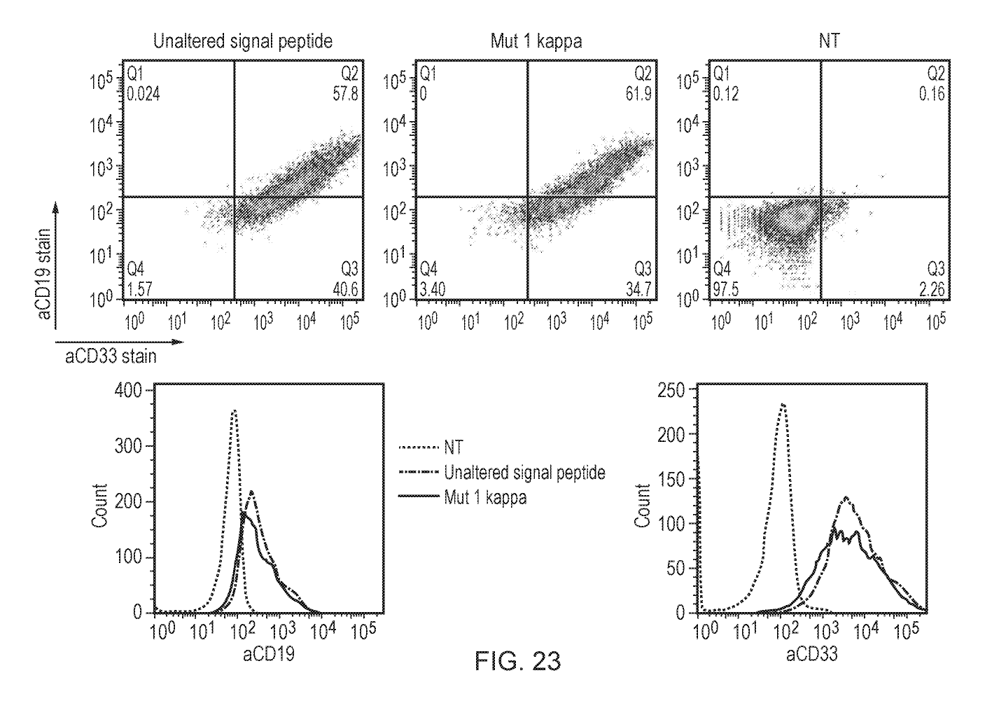

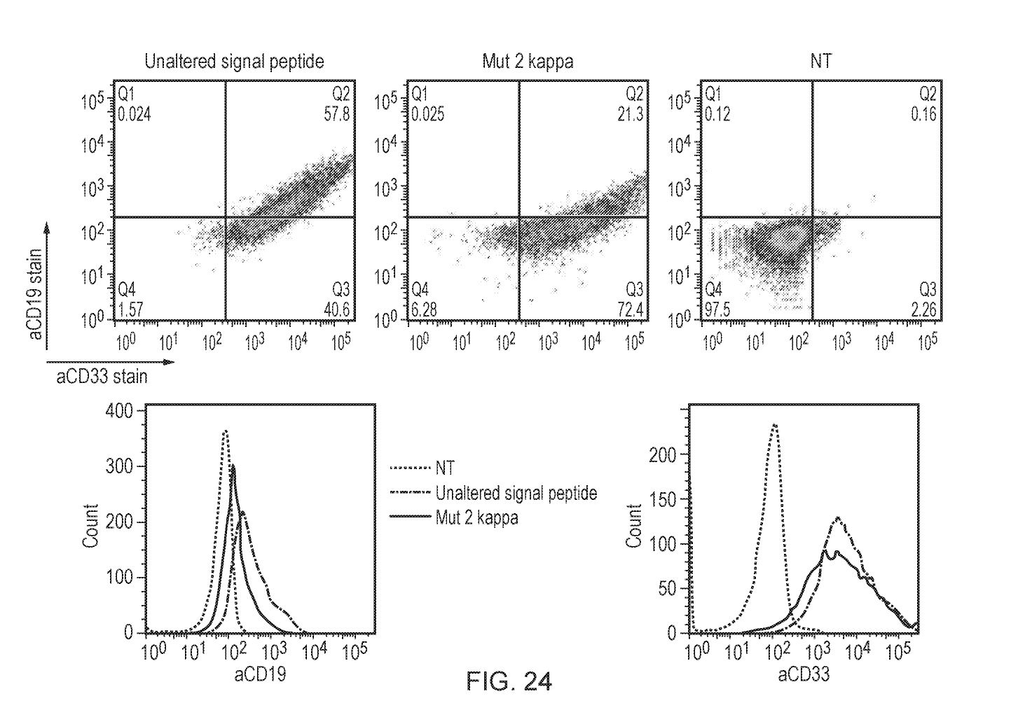

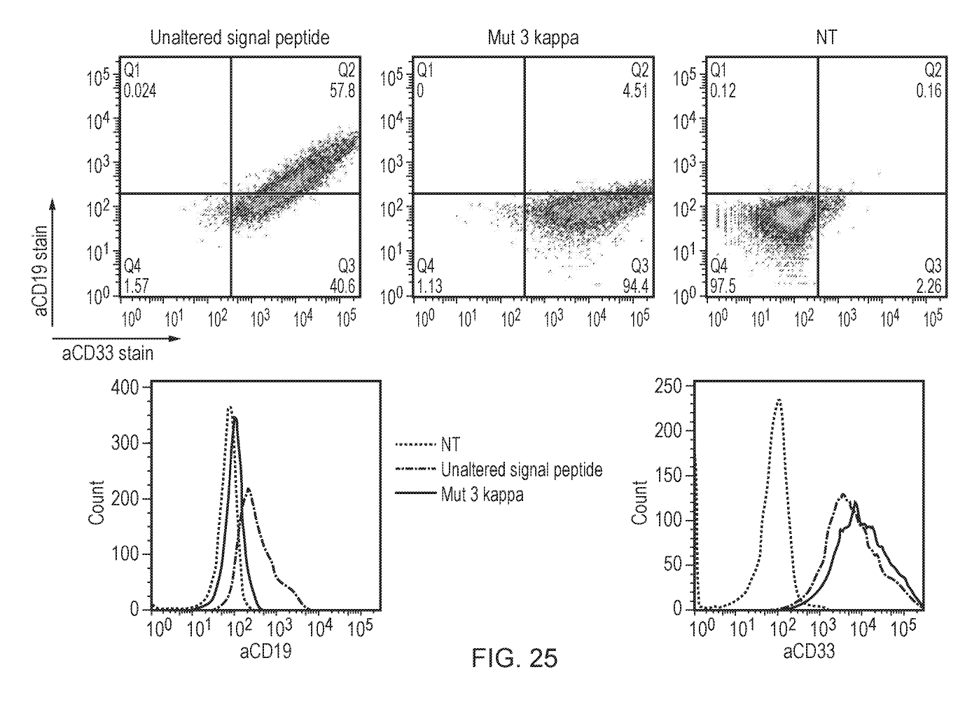

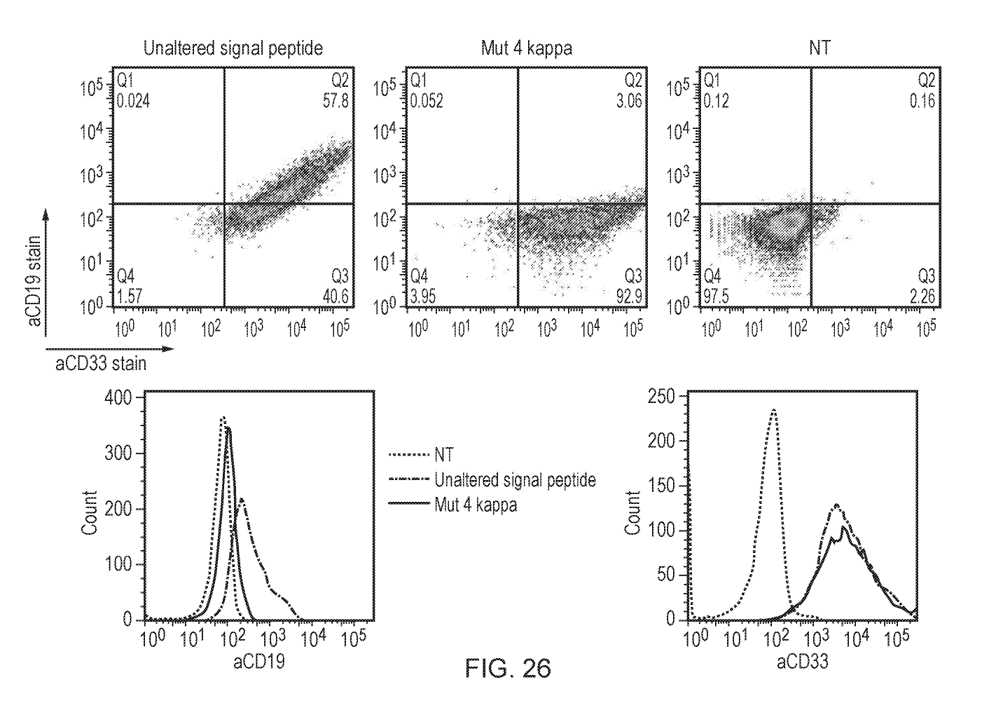

CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcϵR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

CAR-encoding nucleic acids may be transferred to T cells using, for example, retroviral vectors. Lentiviral vectors may be employed. In this way, a large number of cancer-specific T cells can be generated for adoptive cell transfer. When the CAR binds the target-antigen, this results in the transmission of an activating signal to the T-cell it is expressed on. Thus the CAR directs the specificity and cytotoxicity of the T cell towards tumour cells expressing the targeted antigen.

The inventors have defined three distinct categories of cells which co-express a first CAR and a second CAR such that the cell can recognize a desired pattern of expression on target cells in the manner of a logic gate as detailed in the truth tables: Table 1, 2 and 3.

Both the first and second (and optionally subsequent) CARs comprise:
(i) an antigen-binding domain;
(ii) a spacer;
(iii) a transmembrane domain; and
(iii) an intracellular domain, which may comprise or associate with a T-cell signalling endodomain.

TABLE 1
Truth Table for CAR OR GATE

| Antigen A | Antigen B | Response |
|---|---|---|
| Absent | Absent | No activation |
| Absent | Present | Activation |
| Present | Absent | Activation |
| Present | Present | Activation |

TABLE 2
Truth Table for CAR AND GATE

| Antigen A | Antigen B | Response |
|---|---|---|
| Absent | Absent | No activation |
| Absent | Present | No Activation |
| Present | Absent | No Activation |
| Present | Present | Activation |

TABLE 3
Truth Table for CAR AND NOT GATE

| Antigen A | Antigen B | Response |
|---|---|---|
| Absent | Absent | No activation |
| Absent | Present | No Activation |
| Present | Absent | Activation |
| Present | Present | No Activation |

Using the nucleic acid construct of the present invention, the first and second CARs are produced as a polypeptide comprising both CARs, together with a cleavage site.

SEQ ID No. 4 and 5 are examples of AND gates, which comprise two CARs. The nucleic acid construct of the invention may comprise one or other part of the following amino acid sequences, which corresponds to a single CAR. One or both CAR sequences may be modified to include one or more intracellular retention signals, and/or to alter their signal peptides, as defined below.

SEQ ID No 4 is a CAR AND gate which recognizes CD19 AND CD33 using a CD148 phosphatase

SEQ ID No 5 is an alternative implementation of the CAR AND GATE which recognizes CD19 AND CD33 which uses a CD45 phosphatase.

SEQ ID No. 4
MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPTQVLGLLLLWLTDARCDI
QMTQSPSSLSASVGDRVTITCRASEDIYFNLVWYQQKPGKAPKLLIYDTN
RLADGVPSRFSGSGSGTQYTLTISSLQPEDFATYYCQHYKNYPLTFGQGT
KLEIKRSGGGGSGGGGSGGGGSGGGGSRSEVQLVESGGGLVQPGGSLRLS
CAASGFTLSNYGMHWIRQAPGKGLEWVSSISLNGGSTYYRDSVKGRFTIS
RDNAKSTLYLQMNSLRAEDTAVYYCAAQDAYTGGYFDYWGQGTLVTVSSM
DPAEPKSPDKTHTCPPCPAPPVAGPSVFLFPPKPKDTLMIARTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPGKKDPKAVFGCIFGALVI
VTVGGFIFWRKKRKDAKNNEVSFSQIKPKKSKLIRVENFEAYFKKQQADS
NCGFAEEYEDLKLVGISQPKYAAELAENRGKNRYNNVLPYDISRVKLSVQ
THSTDDYINANYMPGYHSKKDFIATQGPLPNTLKDFWRMVWEKNVYAIIM
LTKCVEQGRTKCEEYWPSKQAQDYGDITVAMTSEIVLPEWTIRDFTVKNI
QTSESHPLRQFHFTSWPDHGVPDTTDLLINFRYLVRDYMKQSPPESPILV
HCSAGVGRTGTFIAIDRLIYQIENENTVDVYGIVYDLRMHRPLMVQTEDQ
YVFLNQCVLDIVRSQKDSKVDLIYQNTTAMTIYENLAPVTTFGKTNGYIA

SEQ ID No. 4 breaks down as follows:

Signal peptide derived from Human CD8a:
MSLPVTALLLPLALLLHAARP
scFv aCD19:
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
Linker:
SD
Human CD8aSTK:
PTTTPAPRPPTPAPTIASQPLSLRPEACRPAAGGAVHTRGLDFACDI
Human CD28TM:
FWVLVVVGGVLACYSLLVTVAFIIFWV
Human CD3zeta intracellular domain:
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A peptide:
RAEGRGSLLTCGDVEENPGP
Signal peptide derived from mouse Ig kappa:
MAVPTQVLGLLLLWLTDA
scFv aCD33:
RCDIQMTQSPSSLSASVGDRVTITCRASEDIYFNLVWYQQKPGKAPKLLI
YDTNRLADGVPSRFSGSGSGTQYTLTISSLQPEDFATYYCQHYKNYPLTF
GQGTKLEIKRSGGGGSGGGGSGGGGSGGGGSRSEVQLVESGGGLVQPGGS
LRLSCAASGFTLSNYGMHWIRQAPGKGLEWVSSISLNGGSTYYRDSVKGR
FTISRDNAKSTLYLQMNSLRAEDTAVYYCAAQDAYTGGYFDYWGQGTLVT
VSSM
Linker:
DPA
Hinge and Fc derived from human IgG1 with
mutations to prevent FcRg association
(HCH2CH3pvaa):
EPKSPDKTHTCPPCPAPPVAGPSVFLFPPKPKDTLMIARTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
Linker:
KDPK
Human CD148TM:
AVFGCIFGALVIVTVGGFIFW
Human CD148 intracellular domain:
RKKRKDAKNNEVSFSQIKPKKSKLIRVENFEAYFKKQQADSNCGFAEEYE
DLKLVGISQPKYAAELAENRGKNRYNNVLPYDISRVKLSVQTHSTDDYIN
ANYMPGYHSKKDFIATQGPLPNTLKDFWRMVWEKNVYAIIMLTKCVEQGR
TKCEEYWPSKQAQDYGDITVAMTSEIVLPEWTIRDFTVKNIQTSESHPLR
QFHFTSWPDHGVPDTTDLLINFRYLVRDYMKQSPPESPILVHCSAGVGRT
GTFIAIDRLIYQIENENTVDVYGIVYDLRMHRPLMVQTEDQYVFLNQCVL
DIVRSQKDSKVDLIYQNTTAMTIYENLAPVTTFGKTNGYIA
SEQ ID No. 5
MSLPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITKAGGGGSGGGGSGGGGSGGGGS
EVKLQESGPGLVAPSQSLSVTCTVSGVSLPDYGVSWIRQPPRKGLEWLGV
IWGSETTYYNSALKSRLTIIKDNSKSQVFLKMNSLQTDDTAIYYCAKHYY
YGGSYAMDYWGQGTSVTVSSDPTTTPAPRPPTPAPTIASQPLSLRPEACR
PAAGGAVHTRGLDFACDIFWVLVVVGGVLACYSLLVTVAFIIFWVRRVKF
SRSADAPAYQQGQNQLYNELNLGRREEYDVLDKRRGRDPEMGGKPRRKNP
QEGLYNELQKDKMAEAYSEIGMKGERRRGKGHDGLYQGLSTATKDTYDAL
HMQALPPRRAEGRGSLLTCGDVEENPGPMAVPTQVLGLLLLWLTDARCDI
QMTQSPSSLSASVGDRVTITCRASEDIYFNLVWYQQKPGKAPKLLIYDTN
RLADGVPSRFSGSGSGTQYTLTISSLQPEDFATYYCQHYKNYPLTFGQGT
KLEIKRSGGGGSGGGGSGGGGSGGGGSRSEVQLVESGGGLVQPGGSLRLS
CAASGFTLSNYGMHWIRQAPGKGLEWVSSISLNGGSTYYRDSVKGRFTIS
RDNAKSTLYLQMNSLRAEDTAVYYCAAQDAYTGGYFDYWGQGTLVTVSSM
DPAEPKSPDKTHTCPPCPAPPVAGPSVFLFPPKPKDTLMIARTPEVTCVV
VDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTVD
KSRWQQGNVFSCSVMHEALHNHYTQKSLSLSPGKKDPKALIAFLAFLIIV
TSIALLVVLYKIYDLHKKRSCNLDEQQELVERDDEKQLMNVEPIHADILL
ETYKRKIADEGRLFLAEFQSIPRVFSKFPIKEARKPFNQNKNRYVDILPY
DYNRVELSEINGDAGSNYINASYIDGFKEPRKYIAAQGPRDETVDDFWRM
IWEQKATVIVMVTRCEEGNRNKCAEYWPSMEEGTRAFGDVVVKINQHKRC
PDYIIQKLNIVNKKEKATGREVTHIQFTSWPDHGVPEDPHLLLKLRRRVN
AFSNFFSGPIVVHCSAGVGRTGTYIGIDAMLEGLEAENKVDVYGYVVKLR
RQRCLMVQVEAQYILIHQALVEYNQFGETEVNLSELHPYLHNMKKRDPPS
EPSPLEAEFQRLPSYRSWRTQHIGNQEENKSKNRNSNVIPYDYNRVPLKH
ELEMSKESEHDSDESSDDDSDSEEPSKYINASFIMSYWKPEVMIAAQGPL
KETIGDFWQMIFQRKVKVIVMLTELKHGDQEICAQYWGEGKQTYGDIEVD
LKDTDKSSTYTLRVFELRHSKRKDSRTVYQYQYTNWSVEQLPAEPKELIS
MIQVVKQKLPQKNSSEGNKHHKSTPLLIHCRDGSQQTGIFCALLNLLESA
ETEEVVDIFQVVKALRKARPGMVSTFEQYQFLYDVIASTYPAQNGQVKKN
NHQEDKIEFDNEVDKVKQDANCVNPLGAPEKLPEAKEQAEGSEPTSGTEG
PEHSVNGPASPALNQGS

SEQ ID No. 5 breaks down as follows:

Signal peptide derived from Human CD8a:
MSLPVTALLLPLALLLHAARP
scFv aCD19:
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITKAGGGGSGGGGSGGGGSGGGGSEVKLQESGPGLVAPSQSLSVT
CTVSGVSLPDYGVSWIRQPPRKGLEWLGVIWGSETTYYNSALKSRLTIIK
DNSKSQVFLKMNSLQTDDTAIYYCAKHYYYGGSYAMDYWGQGTSVTVS
Linker:
SD
Human CD8aSTK:
PTTTPAPRPPTPAPTIASQPLSLRPEACRPAAGGAVHTRGLDFACDI
Human CD28TM:
FWVLVVVGGVLACYSLLVTVAFIIFWV
Human CD3zeta intracellular domain:
RRVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATKD
TYDALHMQALPPR
2A peptide:
RAEGRGSLLTCGDVEENPGP
Signal peptide derived from mouse Ig kappa:
MAVPTQVLGLLLLWLTDA
scFv aCD33:
RCDIQMTQSPSSLSASVGDRVTITCRASEDIYFNLVWYQQKPGKAPKLLI
YDTNRLADGVPSRFSGSGSGTQYTLTISSLQPEDFATYYCQHYKNYPLTF
GQGTKLEIKRSGGGGSGGGGSGGGGSGGGGSRSEVQLVESGGGLVQPGGS
LRLSCAASGFTLSNYGMHWIRQAPGKGLEWVSSISLNGGSTYYRDSVKGR
FTISRDNAKSTLYLQMNSLRAEDTAVYYCAAQDAYTGGYFDYWGQGTLVT
VSSM
Linker:
DPA
Hinge and Fc derived from human IgG1 with
mutations to prevent FcRg association
(HCH2CH3pvaa):
EPKSPDKTHTCPPCPAPPVAGPSVFLFPPKPKDTLMIARTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
Linker:
KDPK
Human CD45TM:
ALIAFLAFLIIVTSIALLVVLY
Human CD45 intracellular domain:
KIYDLHKKRSCNLDEQQELVERDDEKQLMNVEPIHADILLETYKRKIADE
GRLFLAEFQSIPRVFSKFPIKEARKPFNQNKNRYVDILPYDYNRVELSEI
NGDAGSNYINASYIDGFKEPRKYIAAQGPRDETVDDFWRMIWEQKATVIV
MVTRCEEGNRNKCAEYWPSMEEGTRAFGDVVVKINQHKRCPDYIIQKLNI
VNKKEKATGREVTHIQFTSWPDHGVPEDPHLLLKLRRRVNAFSNFFSGPI
VVHCSAGVGRTGTYIGIDAMLEGLEAENKVDVYGYVVKLRRQRCLMVQVE
AQYILIHQALVEYNQFGETEVNLSELHPYLHNMKKRDPPSEPSPLEAEFQ
RLPSYRSWRTQHIGNQEENKSKNRNSNVIPYDYNRVPLKHELEMSKESEH
DSDESSDDDSDSEEPSKYINASFIMSYWKPEVMIAAQGPLKETIGDFWQM
IFQRKVKVIVMLTELKHGDQEICAQYWGEGKQTYGDIEVDLKDTDKSSTY
TLRVFELRHSKRKDSRTVYQYQYTNWSVEQLPAEPKELISMIQVVKQKLP
QKNSSEGNKHHKSTPLLIHCRDGSQQTGIFCALLNLLESAETEEVVDIFQ
VVKALRKARPGMVSTFEQYQFLYDVIASTYPAQNGQVKKNNHQEDKIEFD
NEVDKVKQDANCVNPLGAPEKLPEAKEQAEGSEPTSGTEGPEHSVNGPAS
PALNQGS

The present invention relates to the modulation of the relative expression of the two or more CARs in an AND gate. This may be done, for example, by including an intracellular retention signal in one or other CAR.

In relation to SEQ ID NO. 4 and 5 given above, a suitable position for the intracellular retention signal may be readily determined based on the position of the retention signal, or signals of a similar type, in its native protein. The modulatory effect of the retention signal may also be fine tuned by choosing a certain position for the retention signal in the molecule.

For example, the nucleic acid construct may comprise a tyrp-1 retention protein sequence, such as:

(SEQ ID No. 6)
RARRSMDEANQPLLTDQYQCYAEEYEKLQNPNQSVV

Such a sequence may be included, for example, in the CD19 CAR sequence in order for the relative expression level of CD19 CAR to be reduced with respect to CD33 CAR.

The position of the tyrp-1 retention signal in the aCD19 receptor will alter the amount of reduction: for low expression levels, the tyrp-1 retention signal may be placed between “Human CD28TM” and “Human CD3zeta intracellular domain”; for medium expression levels the tyrp-1 retention signal may be placed between “Human CD3zeta intracellular domain” and “2A peptide” in SEQ ID NO. 4 or 5.

Alternatively, the nucleic acid construct may comprise the Adenoviral E3/19K intracellular retention signal. The intracellular retention signal may comprise the E3/19K cytosolic domain KYKSRRSFIDEKKMP (SEQ ID No. 2) or a portion thereof, such as the sequence DEKKMP (SEQ ID No. 3).

The E3/19K retention signal may be positioned at the C-terminus of the CAR whose expression is to be reduced.

The nucleic acid construct of the invention may encode SEQ ID No. 4 or 5, or any of their components parts, such as the CD19 CAR or the CD33 CAR.

The nucleic acid construct of the invention may encode a variant of the CAR-encoding part of the sequence shown as SEQ ID No. 4 or 5 having at least 80, 85, 90, 95, 98 or 99% sequence identity, provided that the variant sequence is a CAR having the required properties.

Methods of sequence alignment are well known in the art and are accomplished using suitable alignment programs. The % sequence identity refers to the percentage of amino acid or nucleotide residues that are identical in the two sequences when they are optimally aligned. Nucleotide and protein sequence homology or identity may be determined using standard algorithms such as a BLAST program (Basic Local Alignment Search Tool at the National Center for Biotechnology Information) using default parameters, which is publicly available at http://blast.ncbi.nlm.nih.gov. Other algorithms for determining sequence identity or homology include: LALIGN (http://www.ebi.ac.uk/Tools/psa/laliqn/ and http:www.ebi.ac.uk/Tools/psa/lalign/nucleotide.html), AMAS (Analysis of Multiply Aligned Sequences, at http://www.compbio.dundee.ac.uk/Software/Amas/amas.html), FASTA (http://www.ebi.ac.uk/Tools/sss/fasta/) Clustal Omega (http://www.ebi.ac.uk/Tools/msa/clustalo/), SIM (http://web.expasy.org/sim/), and EMBOSS Needle (http://www.ebi.ac.uk/Tools/psa/emboss_needle/nucleotide.html).

### Kinetic Segregation Model

Subsequent pairing of CARs to generate the AND gate and the AND NOT gate are based on the kinetic segregation model (KS) of T-cell activation. This is a functional model, backed by experimental data, which explains how antigen recognition by a T-cell receptor is converted into down-stream activation signals. Briefly: at the ground state, the signalling components on the T-cell membrane are in dynamic homeostasis whereby dephosphorylated ITAMs are favoured over phosphorylated ITAMs. This is due to greater activity of the transmembrane CD45/CD148 phosphatases over membrane-tethered kinases such as Ick. When a T-cell engages a target cell through a T-cell receptor (or CAR) recognition of cognate antigen, tight immunological synapses form. This close juxtapositioning of the T-cell and target membranes excludes CD45/CD148 due to their large ectodomains which cannot fit into the synapse. Segregation of a high concentration of T-cell receptor associated ITAMs and kinases in the synapse, in the absence of phosphatases, leads to a state whereby phosphorylated ITAMs are favoured. ZAP70 recognizes a threshold of phosphorylated ITAMs and propagates a T-cell activation signal. This advanced understanding of T-cell activation is exploited by the present invention. In particular, the invention is based on this understanding of how ectodomains of different length and/or bulk and/or charge and/or configuration and/or glycosylation result in differential segregation upon synapse formation.

The Car Logical and Gate

In the AND gate, one CAR comprises an activating endodomain and one CAR comprises an inhibitory endodomain whereby the inhibitory CAR constitutively inhibits the first activating CAR, but upon recognition of its cognate antigen releases its inhibition of the activating CAR. In this manner, a T-cell can be engineered to trigger only if a target cell expresses both cognate antigens. This behaviour is achieved by the activating CAR comprising an activating endodomain containing ITAM domains for example the endodomain of CD3 Zeta, and the inhibitory CAR comprising the endodomain from a phosphatase able to dephosphorylate an ITAM (e.g. CD45 or CD148). Crucially, the spacer domains of both CARs are significantly different in size and/or shape and/or charge etc. When only the activating CAR is ligated, the inhibitory CAR is in solution on the T-cell surface and can diffuse in and out of the synapse inhibiting the activating CAR. When both CARs are ligated, due to differences in spacer properties, the activating and inhibiting CAR are differentially segregated allowing the activating CAR to trigger T-cell activation unhindered by the inhibiting CAR.

This is of considerable utility in the field of cancer therapy. Currently, immunotherapies typically target a single antigen. Most cancers cannot be differentiated from normal tissues on the basis of a single antigen. Hence, considerable “on-target off-tumour” toxicity occurs whereby normal tissues are damaged by the therapy. For instance, whilst targeting CD20 to treat B-cell lymphomas with Rituximab, the entire normal B-cell compartment is depleted. For instance, whilst targeting CD52 to treat chronic lymphocytic leukaemia, the entire lymphoid compartment is depleted. For instance, whilst targeting CD33 to treat acute myeloid leukaemia, the entire myeloid compartment is damaged etc. By restricting activity to a pair of antigens, much more refined targeting, and hence less toxic therapy can be developed. A practical example is targeting of CLL which expresses both CD5 and CD19. Only a small proportion of normal B-cells express both antigens, so the off-target toxicity of targeting both antigens with a logical AND gate is substantially less than targeting each antigen individually.

The design of the present invention is a considerable improvement on previous implementation as described by Wilkie et al. ((2012). J. Clin. Immunol. 32, 1059-1070) and then tested in vivo (Kloss et al (2013) Nat. Biotechnol. 31, 71-75). In this implementation, the first CAR comprises of an activating endodomain, and the second a co-stimulatory domain. This way, a T-cell only receives an activating and co-stimulatory signal when both antigens are present. However, the T-cell still will activate in the sole presence of the first antigen resulting in the potential for off-target toxicity. Further, the implementation of the present invention allows for multiple compound linked gates whereby a cell can interpret a complex pattern of antigens.

TABLE 4

| Cancer Type | Antigens |
|---|---|
| Chronic Lymphocytic Leukaemia | CD5, CD19 |
| Neuroblastoma | ALK, GD2 |
| Glioma | EGFR, Vimentin |
| Multiple myeloma | BCMA, CD138 |
| Renal Cell Carcinoma | Carbonic anhydrase IX, G250 |
| T-ALL | CD2, N-Cadherin |
| Prostate Cancer | PSMA, hepsin (or others) |

Differential Expression of Target Antigens

The AND gate of the present invention triggers in the presence of at least two antigens, for example in the presence of one of the antigen pairs listed in Table 4. It is known that for some antigen pairs, the relative levels of expression of the antigens on the cancer cell can be very different. In these circumstances, the present invention enables the relative levels of expression of each antigen-specific CAR to be modulated in order to reflect the relative level of expression of the target antigen.

For example, Cabezudo et al (1999 Haematologica 84:413) investigated the expression of CD5 and CD19 in cells from a variety of B-cell disorders, including chronic lymphocytic leukemia (CLL), B-cell prolymphocytic leukemia (PLL), splenic lymphoma with villous lymphocytes (SLVL) and mantle-cell (Mc) lymphoma in leukemic phase. It was found that for all the B-cell disorders investigated, the levels of expression of CD5 was 2-8 fold greater than the level of expression of CD19.

In the AND gate of the present invention, the relative expression of the CD19 CAR and CD5 CAR may be modified in order to reflect the relative levels of CD19 and CD5 on the cancer cells. For example, the level of expression of the CD19 CAR may be reduced by incorporation of an intracellular retention signal or by altering the signal peptide such that it has fewer hydrophobic amino acids than the signal peptide of the CD5 CAR.

Compound Gates

The kinetic segregation model with the above components allows compound gates to be made e.g. a T-cell which triggers in response to patterns of more than two target antigens. For example, it is possible to make a T cell which only triggers when three antigens are present (A AND B AND C). Here, a cell expresses three CARs, each recognizing antigens A, B and C. One CAR is excitatory and two are inhibitory, which each CAR having spacer domains which result in differential segregation. Only when all three are ligated, will the T-cell activate. A further example: (A OR B) AND C: here, CARs recognizing antigens A and B are activating and have spacers which co-localise, while CAR recognizing antigen C is inhibitory and has a spacer which results in different co-segregation.

Signal Peptide

The polypeptides A and B (and optionally others, C, D etc) encoded by the nucleic acid construct of the invention each comprise may a signal sequence so that when the polypeptide is expressed inside a cell the nascent protein is directed to the endoplasmic reticulum (ER) (see FIG. 20).

The term “signal peptide” is synonymous with “signal sequence”.

A signal peptide is a short peptide, commonly 5-30 amino acids long, present at the N-terminus of the majority of newly synthesized proteins that are destined towards the secretory pathway. These proteins include those that reside either inside certain organelles (for example, the endoplasmic reticulum, golgi or endosomes), are secreted from the cell, and transmembrane proteins.

Signal peptides commonly contain a core sequence which is a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

The signal peptide is commonly positioned at the amino terminus of the molecule, although some carboxy-terminal signal peptides are known.

As mentioned above, signal sequences have a tripartite structure, consisting of a hydrophobic core region (h-region) flanked by an n- and c-region. The latter contains the signal peptidase (SPase) consensus cleavage site. Usually, signal sequences are cleaved off co-translationally, the resulting cleaved signal sequences are termed signal peptides.

In the signal peptide from the murine Ig kappa chain V-III region, which has the sequence: METDTLILWVLLLLVPGSTG: the n-region has the sequence METD; the h-region (shown in bold) has the sequence TLILVVVLLLLV; and the c-region has the sequence PGSTG.

In the nucleic acid construct of the present invention, the signal peptides of the two CARs may differ in the number of hydrophobic amino acids, to modulate the relative levels of expression of the CARs at the cell surface.

In the nucleic acid construct of the present invention the signal sequence of the two (or more) polypeptides therefore may differ in their h-regions. One polypeptide (which has higher relative expression) may have a greater number of hydrophobic amino acids in the h-region that the other polypeptide (which has lower relative expression). The signal peptide of the polypeptide with lower relative expression may comprise one or more amino acid mutations, such as substitutions or deletions, of hydrophobic amino acids in the h-region than the signal peptide of the polypeptide with lower relative expression.

The first signal peptide and the second signal peptide may have substantially the same n- and c-regions, but differ in the h-region as explained above. “Substantially the same” indicates that the n- and c-regions may be identical between the first and second signal peptide or may differ by one, two or three amino acids in the n- or c-chain, without affecting the function of the signal peptide.

The hydrophobic amino acids in the core may, for example be: Alanine (A); Valine (V); Isoleucine (I); Leucine (L); Methionine (M); Phenylalanine (P); Tyrosine (Y); or Tryptophan (W).

The hydrophobic acids mutated in order to alter signal peptide efficiency may be any from the above list, in particular: Valine (V); Isoleucine (I); Leucine (L); and Tryptophan (W).

Of the residues in the h-region, one signal peptide (for example, the altered signal peptide) may comprise at least 10%, 20%, 30%, 40% or 50% fewer hydrophobic amino acids than the other signal peptide (for example, the unaltered signal peptide).

Where the h-region comprises 5-15 amino acids, one signal peptide may comprise 1, 2, 3, 4 or 5 more hydrophobic amino acids than the other signal peptide.

The altered signal peptide may comprise 1, 2, 3, 4 or 5 amino acid deletions or substitutions of hydrophobic amino acids. Hydrophobic amino acids may be replaced with non-hydrophobic amino acids, such as hydrophilic or neutral amino acids.

Signal sequences can be detected or predicted using software techniques (see for example, http://www.predisi.de/).

A very large number of signal sequences are known, and are available in databases. For example, http://www.signalpeptide.de lists 2109 confirmed mammalian signal peptides in its database.

Table 5 provides a list of signal sequences purely for illustrative purposes. The hydrophobic core is highlighted in bold. This includes examples of amino acids which may be substituted or removed for the purposes of the present invention.

TABLE 5

| Accession | | | | Signal Sequence |
|---|---|---|---|---|
| Number | Entry Name | Protein Name | Length | (hydrophobic core) |
| P01730 | CD4_HUMAN | T-cell surface | 25 | MNRGVPFRHLLLVLQLALLPAATQG |
| | | glycoprotein CD4 | | |
| P08575 | CD45_HUMAN | Leukocyte common | 23 | MYLWLKLLAFGFAFLDTEVFVTG |
| | | antigen | | |
| P01732 | CD8A_HUMAN | T-cell surface | 21 | MALPVTALLLPLALLLHAARP |
| | | glycoprotein CD8 | | |
| | | alpha chain | | |
| P10966 | CD8B_HUMAN | T-cell surface | 21 | MRPRLWLLLAAQLTVLHGNSV |
| | | glycoprotein CD8 | | |
| | | beta chain | | |
| P06729 | CD2_HUMAN | T-cell surface | 24 | MSFPCKFVASFLLIFNVSSKGAVS |
| | | antigen CD2 | | |
| P06127 | CD5_HUMAN | T-cell surface | 24 | MPMGSLQPLATLYLLGMLVASCLG |
| | | glycoprotein CD5 | | |
| P09564 | CD7_HUMAN | T-cell antigen CD7 | 25 | MAGPPRLLLLPLLLALARGLPGALA |
| P17643 | TYRP1_HUMAN | 5,6-dihydroxyindole- | 24 | MSAPKLLSLGCIFFPLLLFQQARA |
| | | 2-carboxylic acid | | |
| | | oxidase | | |
| P00709 | LALBA_HUMAN | Alpha-lactalbumin | 19 | MRFFVPLFLVGILFPAILA |
| P16278 | BGAL_HUMAN | Beta-galactosidase | 23 | MPGFLVRILPLLLVLLLLGPTRG |
| P31358 | CD52_HUMAN | CAMPATH-1 antigen | 24 | MKRFLFLLLTISLLVMVQIQTGLS |
| Q6YHK3 | CD109_HUMAN | CD109 antigen | 21 | MQGPPLLTAAHLLCVCTAALA |
| P01024 | CO3_HUMAN | Complement C3 | 22 | MGPTSGPSLLLLLLTHLPLALG |
| P10144 | GRAB_HUMAN | Granzyme B | 18 | MQPILLLLAFLLLPRADA |
| P04434 | KV310_HUMAN | Ig kappa chain | 20 | MEAPAQLLFLLLLWLPDTTR |
| | | V-III region VH | | |
| P06312 | KV401_HUMAN | Ig kappa chain | 20 | MVLQTQVFISLLLWISGAYG |
| | | V-IV region | | |
| P06319 | LV605_HUMAN | Ig lambda chain | 19 | MAWAPLLLTLLAHCTDCWA |
| | | V-VI region EB4 | | |
| P31785 | IL2RG_HUMAN | Cytokine receptor | 22 | MLKPSLPFTSLLFLQLPLLGVG |
| | | common gamma chain | | |
| Q8N4F0 | BPIL1_HUMAN | Bactericidal/ | 20 | MAWASRLGLLLALLLPVVGA |
| | | permeability-increasing | | |
| | | protein-like 1 | | |
| P55899 | FCGRN_HUMAN | IgG receptor FcRn | 23 | MGVPRPQPWALGLLLFLLPGSLG |
| | | large subunit p51 | | |

The mutated signal peptide comprises one or more mutation(s) such that it has fewer hydrophobic amino acids than the wild-type signal peptide from which it is derived. The term “wild type” means the sequence of the signal peptide which occurs in the natural protein from which it is derived. For example, the signal peptide described in the examples is the signal peptide from the murine Ig kappa chain V-III region, which has the wild-type sequence: METDTLILVWLLLLVPGSTG.
The term “wild-type” also includes signal peptides derived from a naturally occurring protein which comprise one or more amino acid mutations in the n- or c-region. For example it is common to modify a natural signal peptide with a conserved amino acid substitution on the N-terminus to introduce a restriction site. Such modified signal peptide sequences (which do not comprise any mutations in the h-region) are considered “wild-type” for the purposes of the present invention.
The present invention also relates to synthetic signal peptide sequences, which cannot be defined with reference to a wild-type sequence. In this embodiment, the signal peptide of the one polypeptide comprises fewer hydrophobic amino acids than the signal sequence of the other polypeptide. The two signal sequences may be derived from the same synthetic signal peptide sequence, but differ in the number of hydrophobic amino acids in the core region.

Antigen Binding Domain

The antigen binding domain is the portion of the CAR which recognizes antigen. Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain antibody; an artificial single binder such as a Darpin (designed ankyrin repeat protein); or a single-chain derived from a T-cell receptor.

The antigen binding domain may comprise a domain which is not based on the antigen binding site of an antibody. For example the antigen binding domain may comprise a domain based on a protein/peptide which is a soluble ligand for a tumour cell surface receptor (e.g. a soluble peptide such as a cytokine or a chemokine); or an extracellular domain of a membrane anchored ligand or a receptor for which the binding pair counterpart is expressed on the tumour cell.

The antigen binding domain may be based on a natural ligand of the antigen. For example, the antigen binding domain may comprise APRIL, the natural ligand of BCMA.

The antigen binding domain may comprise an affinity peptide from a combinatorial library or a de novo designed affinity protein/peptide.

Spacer Domain

CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

The first and second CARs encoded by the nucleic acid construct of the invention may comprise different spacer molecules. For example, the spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

Examples of amino acid sequences for these spacers are given below:

SEQ ID No. 7 (hinge-CH2CH3 of human IgG1)
AEPKSPDKTHTCPPCPAPPVAGPSVFLFPPKPKDTLMIARTPEVTCVV
VDVSHEDPEVKFNWYVDGVEVHNAKTKPREEQYNSTYRVVSVLTVLHQ
DWLNGKEYKCKVSNKALPAPIEKTISKAKGQPREPQVYTLPPSRDELT
KNQVSLTCLVKGFYPSDIAVEWESNGQPENNYKTTPPVLDSDGSFFLY
SKLTVDKSRWQQGNVFSCSVMHEALHNHYTQKSLSLSPGKKD
SEQ ID No. 8 (human CD8 stalk):
TTTPAPRPPTPAPTIASQPLSLRPEACRPAAGGAVHTRGLDFACDI
SEQ ID No. 9 (human IgG1 hinge):
AEPKSPDKTHTCPPCPKDPK
SEQ ID No. 10 (CD2 ectodomain)
KEITNALETWGALGQDINLDIPSFQMSDDIDDIKWEKTSDKKKIAQFR
KEKETFKEKDTYKLFKNGTLKIKHLKTDDQDIYKVSIYDTKGKNVLEK
IFDLKIQERVSKPKISWTCINTTLTCEVMNGTDPELNLYQDGKHLKLS
QRVITHKWTTSLSAKFKCTAGNKVSKESSVEPVSCPEKGLD
SEQ ID no. 11 (CD34 ectodomain)
SLDNNGTATPELPTQGTFSNVSTNVSYQETTTPSTLGSTSLHPVSQHG
NEATTNITETTVKFTSTSVITSVYGNTNSSVQSQTSVISTVFTTPANV
STPETTLKPSLSPGNVSDLSTTSTSLATSPTKPYTSSSPILSDIKAEI
KCSGIREVKLTQGICLEQNKTSSCAEFKKDRGEGLARVLCGEEQADAD
AGAQVCSLLLAQSEVRPQCLLLVLANRTEISSKLQLMKKHQSDLKKLG
ILDFTEQDVASHQSYSQKT

Since CARs are typically homodimers (see FIG. 1a), cross-pairing may result in a heterodimeric chimeric antigen receptor. This is undesirable for various reasons, for example: (1) the epitope may not be at the same “level” on the target cell so that a cross-paired CAR may only be able to bind to one antigen; (2) the VH and VL from the two different scFv could swap over and either fail to recognize target or worse recognize an unexpected and unpredicted antigen.

For the AND gate it is important that the spacer of the first CAR has a different length, and/or charge and/or shape and/or configuration and/or glycosylation, such that when both first and second CARs bind their target antigen, the difference in spacer charge or dimensions results in spatial separation of the two types of CAR to different parts of the membrane to result in activation as predicted by the kinetic separation model. In the construct of the present invention, therefore, the different length, shape and/or configuration of the spacers is carefully chosen bearing in mind the size and binding epitope on the target antigen to allow differential segregation upon cognate target recognition. For example the IgG1 Hinge, CD8 stalk, IgG1 Fc, ectodomain of CD34, ectodomain of CD45 are expected to differentially segregate.

Examples of spacer pairs which differentially segregate and are therefore suitable for use with the AND gate are shown in the following Table:

| Stimulatory CAR spacer | Inhibitory CAR spacer |
|---|---|
| Human-CD8STK | Human-IgG-Hinge-CH2CH3 |
| Human-CD3z ectodomain | Human-IgG-Hinge-CH2CH3 |
| Human-IgG-Hinge | Human-IgG-Hinge-CH2CH3 |
| Human-CD28STK | Human-IgG-Hinge-CH2CH3 |
| Human-CD8STK | Human-IgM-Hinge-CH2CH3CD4 |
| Human-CD3z ectodomain | Human-IgM-Hinge-CH2CH3CD4 |
| Human-IgG-Hinge | Human-IgM-Hinge-CH2CH3CD4 |
| Human-CD28STK | Human-IgM-Hinge-CH2CH3CD4 |

The relative dimensions of some commonly used spacers are illustrated in FIG. 13. FIG. 14 shows a matrix of spacer pairs and their suitability for use with an AND gate.

All the spacer domains mentioned above form homodimers. However the mechanism is not limited to using homodimeric receptors and should work with monomeric receptors as long as the spacer is sufficiently rigid. An example of such a spacer is CD2 or truncated CD22.

Transmembrane Domain

The transmembrane domain is the sequence of the CAR that spans the membrane.

A transmembrane domain may be any protein structure which is thermodynamically stable in a membrane. This is typically an alpha helix comprising of several hydrophobic residues. The transmembrane domain of any transmembrane protein can be used to supply the transmembrane portion of the invention. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes synthetic transmembrane components).

The transmembrane domain may be derived from CD28, which gives good receptor stability.

Activating Endodomain

The endodomain is the signal-transmission portion of the CAR. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signaling may be needed. For example, chimeric CD28 and OX40 can be used with CD3-Zeta to transmit a proliferative/survival signal, or all three can be used together.

In the “AND gate” of the present invention, the cell comprises at least two CARs, at least one of which is an activating CAR which comprises or associates with an activating endodomain. An activating endodomain may, for example, comprise the CD3-Zeta endodomain alone, the CD3-Zeta endodomain with that of either CD28 or OX40 or the CD28 endodomain and OX40 and CD3-Zeta endodomain.

Any endodomain which contains an ITAM motif can act as an activating endodomain in this invention. Several proteins are known to contain endodomains with one or more ITAM motifs. Examples of such proteins include the CD3 epsilon chain, the CD3 gamma chain and the CD3 delta chain to name a few. The ITAM motif can be easily recognized as a tyrosine separated from a leucine or isoleucine by any two other amino acids, giving the signature YxxL/I. Typically, but not always, two of these motifs are separated by between 6 and 8 amino acids in the tail of the molecule (YxxL/Ix(6-8)YxxL/I). Hence, one skilled in the art can readily find existing proteins which contain one or more ITAM to transmit an activation signal. Further, given the motif is simple and a complex secondary structure is not required, one skilled in the art can design polypeptides containing artificial ITAMs to transmit an activation signal (see WO 2000063372, which relates to synthetic signalling molecules).

The transmembrane and intracellular T-cell signalling domain (endodomain) of a CAR with an activating endodomain may comprise the sequence shown as SEQ ID No. 12, 13 or 14 or a variant thereof having at least 80% sequence identity.

SEQ ID No. 12 comprising CD28 transmembrane
domain and CD3 Z endodomain
FWVLVVVGGVLACYSLLVTVAFIIFWVRRVKFSRSADAPAYQQGQNQL
YNELNLGRREEYDVLDKRRGRDPEMGGKPRRKNPQEGLYNELQKDKMA
EAYSEIGMKGERRRGKGHDGLYQGLSTATKDTYDALHMQALPPR
SEQ ID No. 13 comprising CD28 transmembrane
domain and CD28 and CD3 Zeta endodomains
FWVLVVVGGVLACYSLLVTVAFIIFWVRSKRSRLLHSDYMNMTPRRPG
PTRKHYQPYAPPRDFAAYRSRVKFSRSADAPAYQQGQNQLYNELNLGR
REEYDVLDKRRGRDPEMGGKPRRKNPQEGLYNELQKDKMAEAYSEIGM
KGERRRGKGHDGLYQGLSTATKDTYDALHMQALPPR
SEQ ID No. 14 comprising CD28 transmembrane
domain and CD28, OX40 and CD3 Zeta endodomains.
FWVLVVVGGVLACYSLLVTVAFIIFWVRSKRSRLLHSDYMNMTPRRPG
PTRKHYQPYAPPRDFAAYRSRDQRLPPDAHKPPGGGSFRTPIQEEQAD
AHSTLAKIRVKFSRSADAPAYQQGQNQLYNELNLGRREEYDVLDKRRG
RDPEMGGKPRRKNPQEGLYNELQKDKMAEAYSEIGMKGERRRGKGHDG
LYQGLSTATKDTYDALHMQALPPR

A variant sequence may have at least 80%, 85%, 90%, 95%, 98% or 99% sequence identity to SEQ ID No. 12, 13 or 14, provided that the sequence provides an effective trans-membrane domain and an effective intracellular T cell signaling domain.

“Ligation-Off” Inhibitory Endodomain

In the AND gate of the present invention, one of the CARs comprises an inhibitory endodomain such that the inhibitory CAR inhibits T-cell activation by the activating CAR in the absence of inhibitory CAR ligation, but does not significantly inhibit T-cell activation by the activating CAR when the inhibitory CAR is ligated. This is termed a “ligation-off” inhibitory endodomain.

In this case, the spacer of the inhibitory CAR is of a different length, charge, shape and/or configuration and/or glycosylation from the spacer of the activating CAR, such that when both receptors are ligated, the difference in spacer dimensions results in isolation of the activating CARs and the inhibitory CARs in different membrane compartments of the immunological synapse, so that the activating endodomain is released from inhibition by the inhibitory endodomain.

The inhibitory endodomains for use in a ligation-off inhibitory CAR may therefore comprise any sequence which inhibits T-cell signaling by the activating CAR when it is in the same membrane compartment (i.e. in the absence of the antigen for the inhibitory CAR) but which does not significantly inhibit T cell signaling when it is isolated in a separate part of the membrane from the inhibitory CAR.

The ligation-off inhibitory endodomain may be or comprise a tyrosine phosphatase, such as a receptor-like tyrosine phosphatase. An inhibitory endodomain may be or comprise any tyrosine phosphatase that is capable of inhibiting the TCR signalling when only the stimulatory receptor is ligated. An inhibitory endodomain may be or comprise any tyrosine phosphatase with a sufficiently fast catalytic rate for phosphorylated ITAMs that is capable of inhibiting the TCR signalling when only the stimulatory receptor is ligated.

For example, the inhibitory endodomain of an AND gate may comprise the endodomain of CD148 or CD45. CD148 and CD45 have been shown to act naturally on the phosphorylated tyrosines up-stream of TCR signalling.

CD148 is a receptor-like protein tyrosine phosphatase which negatively regulates TCR signaling by interfering with the phosphorylation and function of PLCγ1 and LAT.

CD45 present on all hematopoetic cells, is a protein tyrosine phosphatase which is capable of regulating signal transduction and functional responses, again by phosphorylating PLC γ1.

An inhibitory endodomain may comprise all of part of a receptor-like tyrosine phosphatase. The phospatase may interfere with the phosphorylation and/or function of elements involved in T-cell signalling, such as PLCγ1 and/or LAT.

The transmembrane and endodomain of CD45 and CD148 is shown as SEQ ID No. 15 and No. 16 respectively.

SEQ ID 15 - CD45 trans-membrane and endodomain
sequence
ALIAFLAFLIIVTSIALLVVLYKIYDLHKKRSCNLDEQQELVERDDEK
QLMNVEPIHADILLETYKRKIADEGRLFLAEFQSIPRVFSKFPIKEAR
KPFNQNKNRYVDILPYDYNRVELSEINGDAGSNYINASYIDGFKEPRK
YIAAQGPRDETVDDFWRMIWEQKATVIVMVTRCEEGNRNKCAEYWPSM
EEGTRAFGDVVVKINQHKRCPDYIIQKLNIVNKKEKATGREVTHIQFT
SWPDHGVPEDPHLLLKLRRRVNAFSNFFSGPIVVHCSAGVGRTGTYIG
IDAMLEGLEAENKVDVYGYVVKLRRQRCLMVQVEAQYILIHQALVEYN
QFGETEVNLSELHPYLHNMKKRDPPSEPSPLEAEFQRLPSYRSWRTQH
IGNQEENKSKNRNSNVIPYDYNRVPLKHELEMSKESEHDSDESSDDDS
DSEEPSKYINASFIMSYWKPEVMIAAQGPLKETIGDFWQMIFQRKVKV
IVMLTELKHGDQEICAQYWGEGKQTYGDIEVDLKDTDKSSTYTLRVFE
LRHSKRKDSRTVYQYQYTNWSVEQLPAEPKELISMIQVVKQKLPQKNS
SEGNKHHKSTPLLIHCRDGSQQTGIFCALLNLLESAETEEVVDIFQVV
KALRKARPGMVSTFEQYQFLYDVIASTYPAQNGQVKKNNHQEDKIEFD
NEVDKVKQDANCVNPLGAPEKLPEAKEQAEGSEPTSGTEGPEHSVNGP
ASPALNQGS
SEQ ID 16 - CD148 trans-membrane and endodomain
sequence
AVFGCIFGALVIVTVGGFIFWRKKRKDAKNNEVSFSQIKPKKSKLIRV
ENFEAYFKKQQADSNCGFAEEYEDLKLVGISQPKYAAELAENRGKNRY
NNVLPYDISRVKLSVQTHSTDDYINANYMPGYHSKKDFIATQGPLPNT
LKDFWRMVWEKNVYAIIMLTKCVEQGRTKCEEYWPSKQAQDYGDITVA
MTSEIVLPEWTIRDFTVKNIQTSESHPLRQFHFTSWPDHGVPDTTDLL
INFRYLVRDYMKQSPPESPILVHCSAGVGRTGTFIAIDRLIYQIENEN
TVDVYGIVYDLRMHRPLMVQTEDQYVFLNQCVLDIVRSQKDSKVDLIY
QNTTAMTIYENLAPVTTFGKTNGYIA

An inhibitory CAR may comprise all or part of SEQ ID No 15 or 16 (for example, it may comprise the phosphatase function of the endodomain). It may comprise a variant of the sequence or part thereof having at least 80%, 85%, 90% or 95% sequence identity, as long as the variant retains the capacity to basally inhibit T cell signalling by the activating CAR.

Other spacers and endodomains may be tested for example using the model system exemplified herein. Target cell populations can be created by transducing a suitable cell line such as a SupT1 cell line either singly or doubly to establish cells negative for both antigens (the wild-type), positive for either and positive for both (e.g. CD19−CD33−, CD19+CD33−, CD19−CD33+ and CD19+CD33+). T cells such as the mouse T cell line BW5147 which releases IL-2 upon activation may be transduced with pairs of CARs and their ability to function in a logic gate measured through measurement of IL-2 release (for example by ELISA). For example, it is shown in Example 4 that both CD148 and CD45 endodomains can function as inhibitory CARs in combination with an activating CAR containing a CD3 Zeta endodomain. These CARs rely upon a short/non-bulky CD8 stalk spacer on one CAR and a bulky Fc spacer on the other CAR to achieve AND gating. When both receptors are ligated, the difference in spacer dimensions results in isolation of the different receptors in different membrane compartments, releasing the CD3 Zeta receptor from inhibition by the CD148 or CD45 endodomains. In this way, activation only occurs once both receptors are activated. It can be readily seen that this modular system can be used to test alternative spacer pairs and inhibitory endodomains. If the spacers do not achieve isolation following ligation of both receptors, the inhibition would not be released and so no activation would occur. If the inhibitory endodomain under test is ineffective, activation would be expected in the presence of ligation of the activating CAR irrespective of the ligation status of the inhibitory CAR.

Cleavage Site

The present nucleic acid construct comprises a sequence encoding a cleavage site positioned between nucleic acid sequences which encode first and second CARs, such that the first and second CARs can be expressed as separate entities.

The cleavage site may be any sequence which enables the polypeptide comprising the first and second CARs to become separated.

The term “cleavage” is used herein for convenience, but the cleavage site may cause the first and second CARs to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode first and second CARs, causes the first and second CARs to be expressed as separate entities.

The cleavage site may be a furin cleavage site.

Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQ\S (where ‘\’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide.

A ‘self-cleaving peptide’ refers to a peptide which functions such that when the polypeptide comprising the first and second CARs and the self-cleaving peptide is produced, it is immediately “cleaved” or separated into distinct and discrete first and second CARs without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus.

The C-terminal 19 amino acids of the longer cardiovirus protein, together with the N-terminal proline of 2B mediate “cleavage” with an efficiency approximately equal to the apthovirus FMDV 2a sequence. Cardioviruses include encephalomyocarditis virus (EMCV) and Theiler's murine encephalitis virus (TMEV).

Mutational analysis of EMCV and FMDV 2A has revealed that the motif DxExNPGP is intimately involved in “cleavage” activity (Donelly et al (2001) as above).

The cleavage site of the present invention may comprise the amino acid sequence:

Dx₁Ex₂NPGP, where x₁ and x₂ are any amino acid. X₁ may be selected from the following group: I, V, M and S. X₂ may be selected from the following group: T, M, S, L, E, Q and F.

For example, the cleavage site may comprise one of the amino acid sequences shown in Table 6.

TABLE 6

| Motif | Present in: |
|---|---|
| DIETNPGP | Picornaviruses EMCB, |
| | EMCD, EMCPV21 |
| DVETNPGP | Picornaviruses MENGO and |
| | TMEBEAN; Insect virus |
| | DCV, ABPV |
| DVEMNPGP | Picornaviruses TMEGD7 and |
| | TMEBEAN |
| DVESNPGP | Picornaviruses FMDA10, |
| | FMDA12, FMDC1, FMD01K, |
| | FMDSAT3, FMDVSAT2, |
| | ERAV; Insect virus CrPV |
| DMESNPGP | Picornavirus FMDV01G |
| DVELNPGP | Picornavirus ERBV; Porcine |
| | rotavirus |
| DVEENPGP | Picornavirus PTV-1; Insect |
| | virus TaV; Trypanosoma |
| | TSR1 |
| DIELNPGP | Bovine Rotavirus, human |
| | rotavirus |
| DIEQNPGP | Trypanosoma AP |
| | endonuclease |
| DSEFNPGP | Bacterial sequence |
| | T. maritima |

The cleavage site, based on a 2A sequence may be, for example 15-22 amino acids in length. The sequence may comprise the C-terminus of a 2A protein, followed by a proline residue (which corresponds to the N-terminal proline of 2B).

Mutational studies have also shown that, in addition to the naturally occurring 2A sequences, some variants are also active. The cleavage site may correspond to a variant sequence from a naturally occurring 2A polypeptide, have one, two or three amino acid substitutions, which retains the capacity to induce the “cleavage” of a polyprotein sequence into two or more separate proteins.

The cleavage sequence may be selected from the following which have all been shown to be active to a certain extent (Donnelly et al (2001) as above):

LLNFDLLKLAGDVESNPGP
LLNFDLLKLAGDVQSNPGP
LLNFDLLKLAGDVEINPGP
LLNFDLLKLAGDVEFNPGP
LLNFDLLKLAGDVESHPGP
LLNFDLLKLAGDVESEPGP
LLNFDLLKLAGDVESQPGP
LLNFDLLKLAGDVESNPGG

Based on the sequence of the DxExNPGP “a motif, “2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al (2001) as above). The cleavage site may comprise one of these 2A-like sequences, such as:

YHADYYKQRLIHDVEMNPGP
HYAGYFADLLIHDIETNPGP
QCTNYALLKLAGDVESNPGP
ATNFSLLKQAGDVEENPGP
AARQMLLLLSGDVETNPGP
RAEGRGSLLTCGDVEENPGP
TRAEIEDELIRAGIESNPGP
TRAEIEDELIRADIESNPGP
AKFQIDKILISGDVELNPGP
SSIIRTKMLVSGDVEENPGP
CDAQRQKLLLSGDIEQNPGP
YPIDFGGFLVKADSEFNPGP

The cleavage site may comprise the 2A-like sequence RAEGRGSLLTCGDVEENPGP.

It has been shown that including an N-terminal “extension” of between 5 and 39 amino acids can increase activity (Donnelly et al (2001) as above). In particular, the cleavage sequence may comprise one of the following sequences or a variant thereof having, for example, up to 5 amino acid changes which retains cleavage site activity:

VTELLYRMKRAETYCPRPLAIHPTEARHKQKIVAPVKQTLNFDLLKL
AGDVESNPGPLLAIHPTEARHKQKIVAPVKQTLNFDLLKLAGDVESN
PGPEARHKQKIVAPVKQTLNFDLLKLAGDVESNPGPAPVKQTLNFDL
LKLAGDVESNPGP

Intracellular Retention Signal

The nucleic acid construct of the present invention may comprise a sequence which encodes a CAR comprising an intracellular retention signal.

Protein targeting or protein sorting is the biological mechanism by which proteins are transported to the appropriate destinations in the cell or outside of it. Proteins can be targeted to the inner space of an organelle, different intracellular membranes, plasma membrane, or to exterior of the cell via secretion. This delivery process is carried out based on sequence information contain in the protein itself.

Proteins synthesised in the rough endoplasmic reticulum (ER) of eukaryotic cells use the exocytic pathway for transport to their final destinations. Proteins lacking special sorting signals are vectorially transported from the ER via the Golgi and the trans-Golgi network (TGN) to the plasma membrane. Other proteins have targeting signals for incorporation into specific organelles of the exocytic pathway, such as endosomes and lysosomes.

Lysosomes are acidic organelles in which endogenous and internalised macromolecules are degraded by luminal hydolases. Endogenous macromolecules reach the lysosome by being sorted in the TGN from which they are transported to endosomes and then lysosomes.

The targeting signals used by a cell to sort proteins to the correct intracellular location may be exploited by the present invention. The signals may be broadly classed into the following types:

i) endocytosis signals
ii) Golgi retention signals
iii) TGN recycling signals
iv) ER retention signals
v) lysosomal sorting signals

‘Intracellular retention signal’ refers to an amino acid sequence which directs the protein in which it is encompassed to a cellular compartment other than the cell surface membrane or to the exterior of the cell.

The intracellular retention signal causes a reduction in the amount of the CAR expressed on the surface of a cell compared to an equivalent, control CAR or other transmembrane protein which does not comprise an intracellular retention signal.

In other words, the proportion of translated CAR comprising an intracellular retention signal which is expressed on at the cell surface is less than the proportion of an equivalent amount of an equivalent, translated control CAR or other transmembrane protein which does not comprise an intracellular retention signal.

For example, the amount of the CAR comprising an intracellular retention signal which is expressed on the surface of a cell may be less than 75%, less than 50%, less than 25% or less than 10% of the amount of an equivalent control CAR or other transmembrane protein which does not comprise an intracellular retention signal.

Constructs which express a polyprotein that is subsequently cleaved by a protease are generally limited by the fact the expression of the peptides from the polyprotein is limited to a 1:1 ratio. However, in the present invention, the inclusion of an intracellular retention signal in the CAR means that its expression on the cell surface can be modulated (e.g. reduced compared to an equivalent control CAR or other transmembrane protein which does not comprise an intracellular retention signal). As such the ratio of the CAR which comprises the intracellular retention signal expressed on the cell surface compared to the expression of the second CAR expressed in the polyprotein may be, for example about 1:1.5, of from 1:1.5-1:2, 1:2-1:3, 1:3-1:4, 1:4-1:5, or more than 1:5.

The amount of a CAR expressed on the surface of a cell may be determined using methods which are known in the art, for example flow cytometry or fluorescence microscopy.

The intracellular retention signal may direct the CAR away from the secretory pathway during translocation from the ER.

The intracellular retention signal may direct the CAR to an intracellular compartment or complex. The intracellular retention signal may direct the CAR to a membrane-bound intracellular compartment.

For example, the intracellular retention signal may direct the CAR to a lysosomal, endosomal or Golgi compartment (trans-Golgi Network, ‘TGN’).

Within a normal cell, proteins arising from biogenesis or the endocytic pathway are sorted into the appropriate intracellular compartment following a sequential set of sorting decisions. At the plasma membrane, proteins can either remain at the cell surface or be internalised into endosomes. At the TGN, the choice is between going to the plasma membrane or being diverted to endosomes. In endosomes, proteins can either recycle to the plasma membrane or go to lysosomes. These decisions are governed by sorting signals on the proteins themselves.

Lysosomes are cellular organelles that contain acid hydrolase enzymes that break down waste materials and cellular debris. The membrane around a lysosome allows the digestive enzymes to work at the pH they require. Lysosomes fuse with autophagic vacuoles (phagosomes) and dispense their enzymes into the autophagic vacuoles, digesting their contents.

An endosome is a membrane-bounded compartment inside eukaryotic cells. It is a compartment of the endocytic membrane transport pathway from the plasma membrane to the lysosome and provides an environment for material to be sorted before it reaches the degradative lysosome. Endosomes may be classified as early endosomes, late endosomes, or recycling endosomes depending on the time it takes for endocytosed material to reach them. The intracellular retention signal used in the present invention may direct the protein to a late endosomal compartment.

The Golgi apparatus is part of the cellular endomembrane system, the Golgi apparatus packages proteins inside the cell before they are sent to their destination; it is particularly important in the processing of proteins for secretion.

There is a considerable body of knowledge which has arisen from studies investigating the sorting signals present in known proteins, and the effect of altering their sequence and/or position within the molecule (Bonifacino and Traub (2003) Ann. Rev. Biochem. 72:395-447; Braulke and Bonifacino (2009) Biochimica and Biophysica Acta 1793:605-614; Griffith (2001) Current Biology 11:R226-R228; Mellman and Nelson (2008) Nat Rev Mol Cell Biol. 9:833-845; Dell'Angelica and Payne (2001) Cell 106:395-398; Schafer et al (1995) EMBO J. 14:2424-2435; Trejo (2005) Mol. Pharmacol. 67:1388-1390). Numerous studies have shown that it is possible to insert one or more sorting signals into a protein of interest in order to alter the intracellular location of a protein of interest (Pelham (2000) Meth. Enzymol. 327:279-283).

It is therefore perfectly possible to select a sorting signal having a desired localisation property and include it within a protein of interest in order to direct the intracellular location of that protein. In connection with the present application, it is therefore possible to select a sorting signal having the desired amount of reduction of expression at the plasma membrane.

The optimal position of the sorting signal in the nascent protein of interest may depend on the type of transmembrane protein (i.e. types I-IV) and whether the C-terminus is on the luminal or the cytoplasmic side of the membrane (Goder and Spiess (2001) FEBS Lett 504:87-93). This may readily be determined by considering the position of the sorting signal in its natural protein.

Examples of endocytosis signals include those from the transferrin receptor and the asialoglycoprotein receptor.

Examples of signals which cause TGN-endosome recycling include those form proteins such as the CI- and CD-MPRs, sortilin, the LDL-receptor related proteins LRP3 and LRP10 and β-secretase, GGA1-3, LIMP-II, NCP1, mucolipn-1, sialin, GLUT8 and invariant chain.

Examples of TGN retention signals include those from the following proteins which are localized to the TGN: the prohormone processing enzymes furin, PC7, CPD and PAM; the glycoprotein E of herpes virus 3 and TGN38.

Examples of ER retention signals include C-terminal signals such as KDEL, KKXX or KXKXX and the RXR(R) motif of potassium channels. Known ER proteins include the adenovirus E19 protein and ERGIC53.

Examples of lysosomal sorting signals include those found in lysosomal membrane proteins, such as LAMP-1 and LAMP-2, CD63, CD68, endolyn, DC-LAMP, cystinosin, sugar phosphate exchanger 2 and acid phosphatase.

Tunability

The relative expression of one or both CARs may be fine tuned using the method of the invention by various methods, such as
a) altering the position of the intracellular retention signal in the protein molecule; and/or
b) selecting a particular intracellular retention signal.

Option a) is discussed in more detail below.

With regard to option b), a range of intracellular retention signals is available from the large number of naturally occurring proteins which are sorted to distinct cellular locations inside eukaryotic cells. It is also possible to use “synthetic” intracellular retention signals which comprise one or more of the motifs found in naturally occurring proteins (see next section) and have a similar sorting signal function.

A cascade of signal strength is available, depending on the intracellular location to which the sorting signal sends the relevant protein. Broadly speaking, the more “intracellular” the location directed by the sorting signal, the “stronger” the signal is in terms of lowering the relative expression of the protein.

When a sorting signal directs a protein to the lysosomal compartment, the protein is internalised and degraded by the cell, resulting in relatively little escape to the cell surface. The protein is degraded and lost from the system once it enters the lysosome. Therefore lysosomal sorting signals, such as LAMP1, are the “strongest” in terms of reducing relative expression at the cell surface.

When a sorting signal directs a protein to be retained in the ER, only a very small proportion of the protein gets to the cell surface. Hence ER retention or recycling signals, such as ER-GIC-53 and KKFF signal are the next most strong, in terms of reducing relative expression at the cell surface.

When a sorting signal directs a protein to the endosomal, Golgi or TGN compartments, then the protein is likely to recycle to some extent between the TGN, the endosomal compartment, and the plasma membrane. These signals provide a more limited level of reduction of expression as a significant proportion of the protein will still reach the plasma membrane.

In general the reduction in expression seen with known sorting signals can be summarised as follows:
Lysosomal sorting signals>ER retention/recycling signals>TGN retention/recycling signals>endocytosis signals.

The tunability using different sorting signals and/or different positions of sorting signals within the protein is especially useful when one considers the expression of multiple proteins, each with their own relative expression. For example, consider a nucleic acid construct having the following structure:

A-X-B-Y-C
in which
   A, B and C are nucleic acid sequences encoding polypeptides; and
   X and Y are nucleic acid sequences encoding cleavage sites.

The nucleic acid construct will encode three proteins A, B and C, any or all of which may be CARs. For example, B and C may be CARs which comprise an intracellular retention signal. If it is desired for A, B and C to be expressed such that the relative levels are A>B>C, then the nucleic acid sequence A may have no intracellular retention signal, the nucleic acid sequence B may have an intracellular retention signal that causes a small proportion of protein B to be retained in the cell (i.e. not to be expressed at the cell surface), and the nucleic acid sequence C may have an intracellular retention signal that causes a large proportion of protein C to be retained in the cell.

As explained below, differential amounts of intracellular retention, leading to different amounts of cell surface expression may be achieved by:

(a) using different intracellular retention signals for the proteins; and/or
(b) having the intracellular retention signal located at a different position in the proteins.

### Signal Types

Numerous proteins which include an intracellular retention signal and are directed to an intracellular compartment are known in the art.

The intracellular retention signal may be a retention signal from a protein which resides in the lysosomal, endosomal or Golgi compartment.

Intracellular retention signals are well known in the art (see, for example, Bonifacino & Traub; Annu. Rev. Biochem.; 2003; 72; 395-447).

The intracellular retention signal may be a tyrosine-based sorting signal, a dileucine-based sorting signal, an acidic cluster signal, a lysosomal avoidance signal, an NPFX′(1,2)D-Type signal, a KDEL, a KKX′X′ or a KX′KX′X′ signal (wherein X′ is any amino acid).

Tyrosine-based sorting signals mediate rapid internalization of transmembrane proteins from the plasma membrane and the targeting of proteins to lysosomes (Bonifacino & Traub; as above). Two types of tyrosine-based sorting signals are represented by the NPX′Y and YX′X′Z′ consensus motifs (wherein Z′ is an amino acid with a bulky hydrophobic side chain).

NPX′Y signals have been shown to mediate rapid internalization of type I transmembrane proteins, they occur in families such as members of the LDL receptor, integrin β, and β-amyloid precursor protein families.

Examples of NPX′Y signals are provided in Table 7.

TABLE 7
NPX′Y signals

| Protein | Species | Sequence |
|---|---|---|
| LDL receptor | Human | Tm-10-INFDNPVYQKTT-29 |
| LRP1 (1) | Human | Tm-21-VEIGNPTYKMYE-64 |
| LRP1 (2) | Human | Tm-55-TNFTNPVYATLY-33 |
| LRP1 | Drosophila | Tm-43-GNFANPVYESMY-38 |
| LRP1 (1) | C. elegans | Tm-54-TTFTNPVYELED-91 |
| LRP1 (2) | C. elegans | Tm-140-LRVDNPLYDPDS-4 |
| Megalin (1) | Human | Tm-70-IIFENPMYSARD-125 |
| Megalin (2) | Human | Tm-144-TNFENPIYAQME-53 |
| Integrin | Human | Tm-18-DTGENPIYKSAV-11 |
| 13-1 (1) | | |
| Integrin | Human | Tm-30-TTVVNPKYEGK |
| 13-1 (2) | | |
| Integrin | Drosophila | Tm-26-WDTENPIYKQAT-11 |
| 13 (1) | | |
| Integtin | Drosophila | Tm-35-STFKNPMYAGK |
| 13 (2) | | |
| APLP1 | Human | Tm-33-HGYENPTYKFLE-3 |
| APP | Human | Tm-32-NCYENPTYKFFE-4 |
| APP-like | Drosophila | Tm-38-NGYENPTYKYFE-3 |
| Insulin | Human | Tm-36-YASSNPEYLSAS-177 |
| receptor | | |
| EGR receptor | Human | Tm-434-GSVQNPVYHNQP-96 |
| (1) | | |
| EGR receptor | Human | Tm-462-TAVGNPEYLNTV-62 |
| (2) | | |
| EGR receptor | Human | Tm-496-ISLDNPDYQQDF-34 |
| (3) | | |

Numbers in parentheses indicate motifs that are present in more than one copy within the same protein. The signals in this and other tables should be considered examples. Key residues are indicated in bold type. Numbers of amino acids before (i.e., amino-terminal) and after (i.e., carboxy-terminal) the signals are indicated.
Abbreviations: Tm, transmembrane; LDL, low density lipoprotein; LRP1, LDL receptor related protein 1; APP, 13-amyloid precursor protein; APLP1, APP-like protein 1.

YX′X′Z′-type signals are found in endocytic receptors such as the transferrin receptor and the asialoglycoprotein receptor, intracellular sorting receptors such as the CI- and CD-MPRs, lysosomal membrane proteins such as LAMP-1 and LAMP-2, and TGN proteins such as TGN38 and furin, as well as in proteins localized to specialized endosomal-lysosomal organelles such as antigen-processing compartments (e.g., HLA-DM) and cytotoxic granules (e.g., GMP-17). The YX′X′Z′-type signals are involved in the rapid internalization of proteins from the plasma membrane. However, their function is not limited to endocytosis, since the same motifs have been implicated in the targeting of transmembrane proteins to lysosomes and lysosome-related organelles.

Examples of YX′X′Z′-type signals are provided in Table 8.

TABLE 8
YX′X′Z′-type signals

| Protein | Species | Sequence |
|---|---|---|
| LAMP-1 | Human | Tm-RKRSHAGYQTI |
| LAMP-2a | Human | Tm-KHHHAGYEQF |
| LAMP-2a | Chicken | Tm-KKHHNTGYEQF |
| LAMP-2b | Chicken | Tm-RRKSRTGYQSV |
| LAMP-2c | Chicken | Tm-RRKSYAGYQTL |
| LAMP | Drosophila | Tm-RRRSTSRGYMSF |
| LAMP | Earthworm | Tm-RKRSRRGYESV |
| CD63 | Human | Tm-KSIRSGYEVM |
| GMP-17 | Human | Tm-HCGGPRPGYETL |
| GMP-17 | Mouse | Tm-HCRTRRAEYETL |
| CD68 | Human | Tm-RRRPSAYQAL |
| CD1b | Human | Tm-RRRSYQNIP |
| CD1c | Human | Tm-KKHCSYQDIL |
| CD1d | Mouse | Tm-RRRSAYQDIR |
| CD1 | Rat | Tm-RKRRRSYQDIM |
| Endolyn | Rat | Tm-KFCKSKERNYHTL |
| Endolyn | Drosophila | Tm-KFYKARNERNYHTL |
| TSC403 | Human | Tm-KIRLRCQSSGYQRI |
| TSC403 | Mouse | Tm-KIRQRHQSSAYQRI |
| Cystinosin | Human | Tm-HFCLYRKRPGYDQLN |
| Putative solute | Human | Tm-12-SLSRGSGYKEI |
| carrier | | |
| TRP-2 | Human | Tm-RRLRKGYTPLMET-11 |
| HLA-DM [symbol] | Human | Tm-RRAGHSSYTPLPGS-9 |
| LmpA | Dictyostelium | Tm-KKLRQQKQQGYQAIINNE |
| Putative lysosomal | Dictyostelium | Tm-RSKSNQNQSYNLIQL |
| protein | | |
| LIMP-II | Dictyostelium | Tm-RKTFYNNNQYNGYNIIN |
| Transferrin receptor | Human | 16-PLSYTRFSLA-35-Tm |
| Asialoglycoprotein | Human | MTKEYQDLQHL-29-Tm |
| receptor H1 | | |
| CI-MPR | Human | Tm-22-SYKYSKVNKE-132 |
| CD-MPR | Human | Tm-40-PAAYRGVGDD-16 |
| CTLA-4 | Human | Tm-10-TGVYVKMPPT-16 |
| Furin | Human | Tm-17-LISYKGLPPE-29 |
| TGN38 | Rat | Tm-23-ASDYQRLNLKL |
| gp41 | HIV-1 | Tm-13-RQGYSPLSFQT-144 |
| Acid phosphatase | Human | Tm-RMQAQPPGYRHVADGEDHA |

See legend to Table 7 for explanation of signal format

Dileucine-based sorting signals ([DE]X′X′X′LL[LI]) play critical roles in the sorting of many type I, type II, and multispanning transmembrane proteins. Dileucine-based sorting signals are involved in rapid internalization and lysosomal degradation of transmembrane proteins and the targeting of proteins to the late endosomal-lysosomal compartments. Transmembrane proteins that contain constitutively active forms of this signal are mainly localised to the late endosomes and lysosomes.

Examples of [DE]X′X′X′LL[LI] sorting signals are provided in Table 9.

TABLE 9
[DE]X′X′X′LL[LI] sorting signals

| Protein | Species | Signal |
|---|---|---|
| CD3-γ | Human | Tm-8-SDKQTLLPN-26 |
| LIMP-II | Rat | Tm-11-DERAPLIRT |
| Nmb | Human | Tm-37-QEKDPLLKN-7 |
| QNR-71 | Quail | Tm-37-TERNPLLKS-5 |
| Pmel17 | Human | Tm-33-GENSPLLSG-3 |
| Tyrosinase | Human | Tm-8-EEKQPLLME-12 |
| Tyrosinase | Medaka fish | Tm-16-GERQPLLQS-13 |
| Tyrosinase | Chicken | Tm-8-PEIQPLLTE-13 |
| TRP-1 | Goldfish | Tm-7-EGRQPLLGD-13 |
| TRP-1 | Human | Tm-7-EANQPLLTD-20 |
| TRP-1 | Chicken | Tm-7-ELHQPLLTD-20 |
| TRP-2 | Zebrafish | Tm-5-REFEPLLNA-11 |
| VMAT2 | Human | Tm-6-EEKMAILMD-29 |
| VMAT1 | Human | Tm-6-EEKLAILSQ-32 |
| VAchT | Mouse | Tm-10-SERDVLLDE-42 |
| VAMP4 | Human | 19-SERRNLLED-88-Tm |
| Neonatal FcR | Rat | Tm-16-DDSGDLLPG-19 |
| CD4 | Human | Tm-12-SQIKRLLSE-17 |
| CD4 | Cat | Tm-12-SHIKRLLSE-17 |
| GLUT4 | Mouse | Tm-17-RRTPSLLEQ-17 |
| GLUT4 | Human | Tm-17-HRTPSLLEQ-17 |
| IRAP | Rat | 46-EPRGSRLLVR-53-Tm |
| Ii | Human | MDDQRDLISNNEQLPMLGR-11-Tm |
| Ii | Mouse | MDDQRDLISNHEQLPILGN-10-Tm |
| Ii | Chicken | MAEEQRDLISSDGSSGVLPI-12-Tm |
| Ii-1 | Zebrafish | MEPDHQNESLIQRVPSAETILGR-12-Tm |
| Ii-2 | Zebrafish | MSSEGNETPLISDQSSVNMGPQN-8-Tm |
| Lamp | Trypanosome | Tm-RPRRRTEEDELLPEEAEGLIDPQN |
| Menkes protein | Human | Tm-74-PDKHSLLVGDFREDDDTAL |
| NPC1 | Human | Tm-13-TERERLLNF |
| AQP4 | Human | Tm-32-VETDDLIL-29 |
| RME-2 | C. elegans | Tm-104-FENDSLL |
| Vam3p | S. cerevisiae | 153-NEQSPLLHN-121-Tm |
| ALP | S. cerevisiae | 7-SEQTRLVP-18-Tm |
| Gap1p | S. cerevisiae | Tm-23-EVDLDLLK-24 |

See legend to Table 7 for explanation of signal format.

DX′X′LL signals constitute a distinct type of dileucine-based sorting signals. These signals are present in several transmembrane receptors and other proteins that cycle between the TGN and endosomes, such as the CI- and CD-MPRs, sortilin, the LDL-receptor-related proteins LRP3 and LRP10, and β-secretase.

Examples of DX′X′LL sorting signals are provided in Table 10.

TABLE 10
DX′X′LL sorting signals

| Protein | Species | Sequence |
|---|---|---|
| CI-MPR | Human | Tm-151-SFHDDSDEDLLHI |
| CI-MPR | Bovine | Tm-150-TFHDDSDEDLLHV |
| CI-MPR | Rabbit | Tm-151-SFHDDSDEDLLNI |
| CI-MPR | Chicken | Tm-148-SFHDDSDEDLLNV |
| CD-MPR | Human | Tm-54-EESEERDDHLLPM |
| CD-MPR | Chicken | Tm-54-DESEERDDHLLPM |
| Sortilin | Human | Tm-41-GYHDDSDEDLLE |
| SorLA | Human | Tm-41-ITGFSDDVPMVIA |
| head- | Hydra | Tm-41-INRFSDDEPLVVA |
| activator BP | | |
| LRP3 | Human | Tm-237-MLEASDDEALLVC |
| ST7 | Human | Tm-330-KNETSDDEALLLC |
| LRP10 | Mouse | Tm-235-WVVEAEDEPLLA |
| LRP10 | Human | Tm-237-WVAEAEDEPLLT |
| Beta- | Human | Tm-9-HDDFADDISLLK |
| secretase | | |
| Mucolipin-1 | Mouse | Tm-43-GRDSPEDHSLLVN |
| Nonclassical | Deer | Tm-6-VRCHPEDDRLLG |
| MHC-I | mouse | |
| FLJ30532 | Human | Tm-83-HRVSQDDLDLLTS |
| GGA1 | Human | 350-ASVSLLDDELMSL-275 |
| GGA1 | Human | 415-ASSGLDDLDLLGK-211 |
| GGA2 | Human | 448-VQNPSADRNLLDL-192 |
| GGA3 | Human | 384-NALSWLDEELLCL-326 |
| GGA | Drosophila | 447-TVDSIDDVPLLSD-114 |

See legend to Table 7 for explanation of signal format.
Serine and threonine residues are underlined.

Another family of sorting motifs is provided by clusters of acidic residues containing sites for phosphorylation by CKII. This type of motif is often found in transmembrane proteins that are localized to the TGN at steady state, including the prohormone-processing enzymes furin, PC6B, PC7, CPD, and PAM, and the glycoprotein E of herpes virus 3.

Examples of acidic cluster signals are provided in Table 11.

TABLE 11
Acidic cluster sorting signals

| Protein | Species | Sequence |
|---|---|---|
| Furin | Mouse | Tm-31-QEECPSDSEEDEG-14 |
| PC6B (1)^{a} | Mouse | Tm-39-RDRDYDEDDEDDI-36 |
| PC6B (2) | Mouse | Tm-69-LDETEDDELEYDDES-4 |
| PC7 | Human | Tm-38-KDPDEVETES-47 |
| CPD | Human | Tm-36-HEFQDETDTEEET-6 |
| PAM | Human | Tm-59-QEKEDDGSESEEEY-12 |
| VMAT2 | Human | Tm-35-GEDEESESD |
| VMAT1 | Human | Tm-35-GEDSDEEPDHEE |
| VAMP4 | Human | 25-LEDDSDEEEDF-81-Tm |
| Glycoprotein B | HCMV | Tm-125-KDSDEEENV |
| Glycoprotein E | Herpes virus 3 | Tm-28-FEDSESTDTEEEF-21 |
| Nef | HIV-1 (AAL65476) | 55-LEAQEEEEV-139 |
| Kex1p (1) | S. cerevisiae | Tm-29-ADDLESGLGAEDDLEQDEQLEG-40 |
| Kex1p (2) | S. cerevisiae | Tm-79-TEIDESFEMTDF |
| Kex2p | S. cerevisiae | Tm-36-TEPEEVEDFDFDLSDEDH-61 |
| Vps10p | S. cerevisiae | Tm-112-FEIEEDDVPTLEEEH-37 |

See legend to Table 7 for explanation of signal format. Serine and threonine residues are underlined.
^{a}The number in parentheses is the motif number.

The KDEL receptor binds protein in the ER-Golgi intermediate compartment, or in the early Golgi and returns them to the ER. Although the common mammalian signal is KDEL, it has been shown that the KDEL receptor binds the sequence HDEL more tightly (Scheel et al; J. Biol. Chem. 268; 7465 (1993)). The intracellular retention signal may be HDEL.

KKX′X′ and KX′KX′X′ signals are retrieval signals which can be placed on the cytoplasmic side of a type I membrane protein. Sequence requirements of these signals are provided in detail by Teasdale & Jackson (Annu. Rev. Cell Dev. Biol.; 12; 27 (1996)).

The intracellular retention signal may be selected from the group of: NPX′Y, YX′X′Z, [DE]X′X′X′L[LI], DX′X′LL, DP[FVV], FX′DX′F, NPF, LZX′Z[DE], LLDLL, PWDLW, KDEL, HDEL, KKX′X′ or KX′KX′X′; wherein X′ is any amino acid and Z′ is an amino acid with a bulky hydrophobic side chain.

The intracellular retention signal may be any sequence shown in Tables 7 to 11.

The intracellular retention signal may comprise the Tyrosinase-related protein (TYRP)-1 intracellular retention signal. The intracellular retention signal may comprise the TYRP-1 intracellular domain. The intracellular retention signal may comprise the sequence NQPLLTD (SEQ ID No. 1).

TYRP1 is a well-characterized melansomal protein which is retained in the melanosome (a specialized lysosome) at >99% efficiency. TYRP1 is a 537 amino acid transmembrane protein with a lumenal domain (1-477aa), a transmembrane domain (478-501), and a cytoplasmic domain (502-537). A di-leucine signal residing on the cytoplasmic domain causes retention of the protein. This di-leucine signal has the sequence shown as SEQ ID No. 1 (NQPLLTD).

The intracellular retention signal may be in the endodomain of the CAR. In other words, the intracellular retention signal may be in the domain of the transmembrane protein which would be on the intracellular side of the cell membrane if the protein was correctly expressed at the cell surface.

The intracellular retention signal may be proximal to the transmembrane domain, for instance being immediately connected to it. The intracellular retention signal may be distal to the transmembrane domain—for instance at the carboxy-terminus of the endodomain. The positioning of the retention signal modulates its activity allowing “tuning” of the relative expression of two proteins. For instance in the case of the TYRP1 di-leucine motif, proximal placement results in low-level surface expression, while distal placement results in intermediate surface expression, as shown in the Examples.

Polypeptide of Interest

Any or all of A or B; or A, B or C of the nucleic acid sequences in the constructs defined herein may encode a CAR which may or may not comprise an intracellular retention signal and/or an altered signal peptide.

The nucleic acid construct may comprise one or more further nucleic acid sequence(s) which encode polypeptide of interest (POIs). For example, the POI(s) may be an intracellular protein such as a nucleic protein, a cytoplasmic protein or a protein localised to a membrane-bound compartment; a secretory protein or a transmembrane protein.

The POI may be a suicide gene and/or a marker gene.

Suicide/Marker Gene

A suicide gene is a gene encoding a polypeptide which, when expressed by a cell enables that cell to be deleted.

A marker gene is a gene encoding a polypeptide which enables selection of a cell expressing that polypeptide.

Various suicide and marker genes are known in the art. WO2013/153391 describes compact polypeptide which comprises both a marker moiety and a suicide moiety. The polypeptide may be co-expressed with a therapeutic transgene, such as a gene encoding a CAR.

The marker moiety comprises a minimal epitope of CD34 which allows efficient selection of transduced cells using, for example, the Miltenyi CD34 cliniMACS system.

The suicide moiety comprises a minimal epitope based on the epitope from CD20. Cells expressing a polypeptide comprising this sequence can be selectively killed using a lytic antibody such as Rituximab.

The combined marker and suicide polypeptide is stably expressed on the cell surface after, for example, retroviral transduction of its encoding sequence.

The marker/suicide polypeptide may have the formula:

St-R1-S1-Q-S2-R2

wherein
St is a stalk sequence which, when the polypeptide is expressed at the surface of a target cell, causes the R and Q epitopes to be projected from the cell surface;
R1 and R2 are a Rituximab-binding epitopes;
S1 and S2 are optional spacer sequences, which may be the same or different; and
Q is a QBEnd10-binding epitope.

The polypeptide may comprise the sequence shown as SEQ ID No.17, or a variant thereof which has at least 80% identity with the sequence shown as SEQ ID No. 17 and which (i) binds QBEND10; (ii) binds Rituximab and (iii) when expressed on the surface of a cell, induces complement-mediated killing of the cell in the presence of Rituximab.

(SEQ ID No. 17)
CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

The present invention provides a method for deleting a cell which expresses such a marker/suicide gene, which comprises the step of exposing the cells to rituximab.

Cell

The present invention relates to a cell which co-expresses a first CAR and a second CAR at the cell surface. The cell expresses a nucleic acid construct according to the first aspect of the invention.

The cell may be any eukaryotic cell capable of expressing a CAR at the cell surface, such as an immunological cell.

In particular the cell may be an immune effector cell such as a T cell or a natural killer (NK) cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytotoxic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The T cell of the invention may be any of the T cell types mentioned above, in particular a CTL.

Natural killer (NK) cells are a type of cytolytic cell which forms part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The CAR cells of the invention may be any of the cell types mentioned above.

CAR-expressing cells, such as CAR-expressing T or NK cells may either be created ex vivo either from a patient's own peripheral blood (1st party), or in the setting of a haematopoietic stem cell transplant from donor peripheral blood (2nd party), or peripheral blood from an unconnected donor (3rd party).

The present invention also provide a cell composition comprising CAR-expressing cells, such as CAR-expressing T and/or NK cells, according to the present invention. The cell composition may be made by transducing a blood-sample ex vivo with a nucleic acid construct according to the present invention.

Alternatively, CAR-expressing cells may be derived from ex vivo differentiation of inducible progenitor cells or embryonic progenitor cells to the relevant cell type, such as T cells. Alternatively, an immortalized cell line such as a T-cell line which retains its lytic function and could act as a therapeutic may be used.

In all these embodiments, CAR cells may be generated by introducing DNA or RNA coding for the CARs by one of many means including transduction with a viral vector, transfection with DNA or RNA.

A CAR T cell of the invention may be an ex vivo T cell from a subject. The T cell may be from a peripheral blood mononuclear cell (PBMC) sample. T cells may be activated and/or expanded prior to being transduced with CAR-encoding nucleic acid, for example by treatment with an anti-CD3 monoclonal antibody.

A CAR T cell of the invention may be made by:
(i) isolation of a T cell-containing sample from a subject or other sources listed above; and
(ii) transduction or transfection of the T cells with a nucleic acid construct encoding the first and second CAR.

The T cells may then by purified, for example, selected on the basis of co-expression of the first and second CAR.

Vector

The present invention also provides a vector which comprises a CAR-encoding nucleic acid construct as defined herein. Such a vector may be used to introduce the nucleic acid sequence(s) into a host cell so that it expresses the first and second CARs.

The vector may, for example, be a plasmid or a viral vector, such as a retroviral vector or a lentiviral vector, or a transposon based vector or synthetic mRNA.

The vector may be capable of transfecting or transducing a T cell.

### PHARMACEUTICAL COMPOSITION

The present invention also relates to a pharmaceutical composition containing a plurality of CAR-expressing cells, such as T cells or NK cells, of the invention. The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

Method of Treatment

The cells of the present invention may be capable of killing target cells, such as cancer cells. The target cell may be recognisable by a defined pattern of antigen expression, for example the expression of antigen A AND antigen B.

The cells of the present invention may be used for the treatment of an infection, such as a viral infection.

The cells of the invention may also be used for the control of pathogenic immune responses, for example in autoimmune diseases, allergies and graft-vs-host rejection.

The cells of the invention may be used for the treatment of a cancerous disease, such as bladder cancer, breast cancer, colon cancer, endometrial cancer, kidney cancer (renal cell), leukemia, lung cancer, melanoma, non-Hodgkin lymphoma, pancreatic cancer, prostate cancer and thyroid cancer.

It is particularly suited for treatment of solid tumours where the availability of good selective single targets is limited.

The cells of the invention may be used to treat: cancers of the oral cavity and pharynx which includes cancer of the tongue, mouth and pharynx; cancers of the digestive system which includes oesophageal, gastric and colorectal cancers; cancers of the liver and biliary tree which includes hepatocellular carcinomas and cholangiocarcinomas; cancers of the respiratory system which includes bronchogenic cancers and cancers of the larynx; cancers of bone and joints which includes osteosarcoma; cancers of the skin which includes melanoma; breast cancer; cancers of the genital tract which include uterine, ovarian and cervical cancer in women, prostate and testicular cancer in men; cancers of the renal tract which include renal cell carcinoma and transitional cell carcinomas of the utterers or bladder; brain cancers including gliomas, glioblastoma multiforme and medullobastomas; cancers of the endocrine system including thyroid cancer, adrenal carcinoma and cancers associated with multiple endocrine neoplasm syndromes; lymphomas including Hodgkin's lymphoma and non-Hodgkin lymphoma; Multiple Myeloma and plasmacytomas; leukaemias both acute and chronic, myeloid or lymphoid; and cancers of other and unspecified sites including neuroblastoma.

Treatment with the cells of the invention may help prevent the escape or release of tumour cells which often occurs with standard approaches.

The invention will now be further described by way of Examples, which are meant to serve to assist one of ordinary skill in the art in carrying out the invention and are not intended in any way to limit the scope of the invention.

### EXAMPLES

### Example 1—Creation of Target Cell Populations

For the purposes of proving the principle of the invention, receptors based on anti-CD19 and anti-CD33 were arbitrarily chosen. Using retroviral vectors, CD19 and CD33 were cloned. These proteins were truncated so that they do not signal and could be stably expressed for prolonged periods. Next, these vectors were used to transduce the SupT1 cell line either singly or doubly to establish cells negative for both antigen (the wild-type), positive for either and positive for both. The expression data are shown in FIG. 3.

### Example 2—Design and Function of the AND Gate

The AND gate combines a simple activating receptor with a receptor which basally inhibits activity, but whose inhibition is turned off once the receptor is ligated. This was achieved by combining a standard 1^{st} generation CAR with a short/non-bulky CD8 stalk spacer and a CD3 Zeta endodomain with a second receptor with a bulky Fc spacer whose endodomain contained either CD148 or CD45 endodomains. When both receptors are ligated, the difference in spacer dimensions results in isolation of the different receptors in different membrane compartments, releasing the CD3 Zeta receptor from inhibition by the CD148 or CD45 endodomains. In this way, activation only occurs once both receptors are activated. CD148 and CD45 were chosen for this as they function in this manner natively: for instance, the very bulky CD45 ectodomain excludes the entire receptor from the immunological synapse. The expression cassette is depicted in FIG. 4 and the subsequent proteins in FIG. 5.

Surface staining for the different specificity showed that both receptor pairs could be effectively expressed on the cell surface shown in FIG. 6. Function in BW5147 shows that the T-cell is only activated in the presence of both antigens (FIG. 7).

### Example 3: Demonstration of Generalizability of the AND Gate

To ensure that the observations were not a manifestation of some specific characteristic of CD19/CD33 and their binders which had been used, the two targeting scFvs were swapped such that now, the activation (ITAM) signal was transmitted upon recognition of CD33, rather than CD19; and the inhibitory (CD148) signal was transmitted upon recognition of CD19, rather than of CD33. Since CD45 and CD148 endodomains are considered to be functionally similar, experimentation was restricted to AND gates with CD148 endodomain. This should still result in a functional AND gate. T-cells expressing the new logic gate where challenged with targets bearing either CD19 or CD33 alone, or both. The T-cells responded to targets expressing both CD19 and CD33, but not to targets expressing only one or none of these antigens. This shows that the AND gate is still functional in this format (FIG. 8B).

On the same lines, it was sought to establish how generalizable our AND gate is: the AND gate should be generalizable across different targets. While there may be lesser or greater fidelity of the gate given relative antigen density, cognate scFv binding kinetics and precise distance of the scFv binding epitope, one would expect to see some AND gate manifestations with a wide set of targets and binders. To test this, three additional AND gates were generated. Once again, experimentation was restricted to the CD148 version of the AND gate. The second scFv from the original CD148 AND gate was replaced with the anti-GD2 scFv huK666, or with the anti-CD5 scFv, or the anti-EGFRvIII scFv MR1.1 to generate the following CAR AND gates: CD19 AND GD2; CD19 AND CD5; CD19 AND EGFRvIII. The following artificial antigen expressing cell lines were also generated: by transducing SupT1, and our SupT1.CD19 with GM3 and GD2 synthases SupT1.GD2 and SupT1.CD19.GD2 were generated. By transducing SupT1 and SupT1.CD19 with a retroviral vector coding for EGFRvIII SupT1.EGFRvIII and SupT1.CD19.EGFRvIII were generated. Since CD5 is expressed on SupT1 cells, a different cell line was used to generate the target cells: 293T cells were generated which express CD19 alone, CD5 alone and both CD5 and CD19 together. Expression was confirmed by flow-cytometry (FIG. 9). T-cells expressing the three new CAR AND gates were challenged with SupT1.CD19 and respective cognate double positive and single positive target cells. All three AND gates demonstrated reduced activation by the double positive cell lines in comparison with the single positive targets (FIG. 10). This demonstrates generalizability of the AND gate design to arbitrary targets and cognate binders.

### Example 4: Experimental Proof of Kinetic Segregation Model of CAR AND Gate

The aim was to prove the model that differential segregation caused by different spacers is the central mechanism behind the ability to generate these logic CAR gates. The model is that if only the activating CAR is ligated, the potent inhibiting ‘ligation off’ type CAR is in solution in the membrane and can inhibit the activating CAR. Once both CARs are ligated, if both CAR spacers are sufficiently different, they will segregate within the synapse and not co-localize. Hence, a key requirement is that the spacers are sufficiently different. If the model is correct, if both spacers are sufficiently similar so they co-localize when both receptors are ligated, the gate will fail to function. To test this, the “bulky” Fc spacer in the original CAR we replaced with a murine CD8 spacer. It was predicted that this has the similar length, bulk and charge as human CD8 but so should not cross-pair with it. Hence, the new gate had a first CAR which recognizes CD19, a human CD8 stalk spacer and an activatory endodomain; while the second CAR recognizes CD33, has a mouse CD8 stalk spacer and a CD148 endodomain (FIG. 8C). T-cells were transduced to express this new CAR gate. These T-cells were then challenged with SupT1 cells expressing CD19 alone, CD33 alone or CD19 and CD33 together. T-cells did not respond to SupT1 cells expressing either antigen alone as per the original AND gate. However, CAR T-cells failed to respond to SupT1 cells expressing both antigens, thereby confirming the model (FIG. 8C). A functional AND gate requires both CARs to have spacers sufficiently different so that they do not co-localize within an immunological synapse (FIGS. 11A and B).

### Example 5: Functional Analysis of the AND Gate in Primary Cells

PBMCs were isolated from blood and stimulated using PHA and IL-2. Two days later the cells were transduced on retronectin coated plates with retro virus containing the CD19:CD33 AND gate construct. On day 5 the expression level of the two CARs translated by the AND gate construct was evaluated via flow cytometry and the cells were depleted of CD56+ cells (predominantly NK cells). On day 6 the PBMCs were placed in a co-culture with target cells at a 1:2 effector to target cell ratio. On day 8 the supernatant was collected and analysed for IFN-gamma secretion via ELISA (FIG. 12).

These data demonstrate that the AND gate functions in primary cells.

### Example 6—Dissection of TYRP1 Lysozomal Retention Signals

The ability of the Tyrosinase-related protein 1 (TYRP1) retention signal to cause retention of a polypeptide when in the context of a more complex endodomain was determined using a number of constructs (FIG. 16). The wild-type construct was compared with constructs where enhanced Green Fluorescent Protein (eGFP) was added or replaced the TYRP1 endodomain. Where eGFP was added, it was placed either after or before the native endodomain so the retention signal was either in its native location (just under the membrane), or distal to it.

All constructs are co-expressed with IRES.CD34. Staining of transduced SupT1 cells is shown with intracellular and surface staining in FIG. 16.

It was found that replacement of the endodomain resulted in very bright surface expression, introduction of eGFP after the retention signal to almost no surface expression and introduction before the retention signal to intermediate surface expression (FIG. 16).

### Example 7—Modulation of the Relative Expression of a Transmembrane Protein Co-Expressed from a Single Expression Cassette with a Separate Protein

An expression cassette encoding two CAR transmembrane proteins was modified such that one of the CAR proteins had the lysozomal retention signal from TYRP1 introduced either proximal or distal to the membrane. Expression of each of these two new variants at the cell surface was compared with that of the original unmodified CAR protein.

PBMCs were isolated from blood and stimulated using PHA and IL-2. Two days later the cells were transduced on retronectin coated plates with retro virus containing the CD19:CD33 CAR construct. On day 5 the expression level of the two CARs translated by the construct was evaluated via flow cytometry and the cells were depleted of CD56+ cells (predominantly NK cells). On day 6 the PBMCs were placed in a co-culture with target cells at a 1:2 effector to target cell ratio. On day 8 the supernatant was collected and analysed for IFN-gamma secretion via ELISA.

The pattern observed with Tyrp1-eGFP fusions was observed with some reduction of expression of modified transmembrane protein with the distal retention signal and marked reduction in the case of proximal retention signal. As expected, expression of the second transmembrane protein from the cassette was not altered (FIG. 17).

### Example 8—Modulation of Expression Using a Retention Signal from the Adenoviral E3/19K Protein

The human adenovirus E3/19K protein is a type I transmembrane glycoprotein of the Endoplasmic Reticulum/Golgi that abrogates cell surface transport of major histocompatibility complex class I (MHC-I) and MHC-I-related chain A and B (MICA/B) molecules. The retention motif was identified to be depended on the cytosolic tail of the adenovirus E3/19K protein. More specifically, the last 6aa DEKKMP was found to be the most important for retention. The optimal positioning was found to be at the c-terminus of the protein.

An expression cassette encoding two CAR transmembrane proteins, as described in Example 7, was modified such that one of the CAR proteins had the retention motif from adenovirus E3/19K protein. In this experiment, the retention motif on the second CAR in the expression cassette (the anti-CD33 inhibitory CAR).

Constructs were generated comprising either the entire cytosolic tail of adenovirus E3/19K protein or only the last 6aa from E3/19K (DEKKMP), which were found to be critical for its Golgi/ER retention ability (FIG. 18). These constructs were transfected into 293T cells and stained primarily with a chimeric soluble CD19-Rabbit Fc and a chimeric soluble CD33-Mouse Fc proteins. These cells were then subsequently stained with an anti-Rabbit Fc-FITC and an anti-Mouse Fc-APC (FIG. 19). These cells show a clear retention when the full length adenovirus E3/19K protein, or the DEKKMP motif, was placed on the anti-CD33 receptor but had no effect on anti-CD19 receptor expression levels.

### Example 9—Modulation of Expression by Altering the Signal Peptide—Swapping in the Murine Ig Kappa Chain V-III Signal Sequence

PCT/GB2014/053452 describes a vector system encoding two chimeric antigen receptors (CARs), one against CD19 and one against CD33. The signal peptide used for the CARs in that study was the signal peptide from the human CD8a signal sequence. For the purposes of this study, this was substituted with the signal peptide from the murine Ig kappa chain V-III region, which has the sequence: METDTLILWVLLLLVPGSTG (hydrophobic residues highlighted in bold). In order to establish that the murine Ig kappa chain V-III signal sequence functioned as well as the signal sequence from human CD8a, a comparative study was performed. For both signal sequences, functional expression of the anti-CD33 CAR and the anti-CD19 CAR was observed. This substituted signal sequence and all subsequent mutations thereof were transiently transfected into 293T cells. Three days after transfection the 293T cells were stained with both soluble chimeric CD19 fused with rabbit Fc chain and soluble chimeric CD33 fused with mouse Fc chain. All cells were then stained with anti-Rabbit Fc-FITC and anti-mouse Fc-APC. Flow cytometry plots show the substituted signal sequence as a comparison with non-transfected (NT) and the construct with Cd8 signal sequences (FIG. 22). The murine Ig kappa chain V-III signal sequence was found to function as well as the signal sequence from human CD8a.

### Example 10—Altering Relative Expression by Deleting Hydrophobic Residues in the Signal Peptide

Hydrophobic residues were deleted in a stepwise fashion and the effect on the relative expression of the anti-CD33 CAR and the anti-CD19 CAR was observed. The effect of one, two, three and four amino acid deletions was investigated and the results are shown in FIGS. 23 to 26 respectively.

All mutant constructs showed a decrease in relative expression of the anti-CD19 CAR compared to the anti-CD33 CAR. The relative decrease of anti-CD19 CAR expression was greater with a greater number of amino acid deletions from 1 to 3, but then plateaued out (four deletions gave a similar decrease in expression as three deletions).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology, cell biology or related fields are intended to be within the scope of the following claims.

1. A nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(i) an antigen-binding domain,
(ii) a spacer,
(iii) a trans-membrane domain, and
(iv) an endodomain
wherein the antigen binding domains of the first and second CARs bind to different antigens, wherein the spacer of the first CAR is different to the spacer of the second CAR, wherein one of the first or second CARs is an activating CAR comprising an activating endodomain and the other CAR is an inhibitory CAR comprising a ligation-off inhibitory endodomain, and wherein:
(a) the first and/or second CAR comprises an intracellular retention signal; and/or
(b) the signal peptide of the first or second CAR comprises one or more mutation(s) such that it has fewer hydrophobic amino acids.

2. The nucleic acid construct according to claim 1, wherein the spacer of the first CAR has a different length and/or charge and/or size and/or configuration and/or glycosylation than the spacer of the second CAR, such that when the nucleic acid construct is expressed in a cell, and the first CAR and the second CAR bind their respective target antigens at the cell surface, the first CAR and second CAR become spatially separated on the cell membrane.

3. (canceled)

4. The nucleic acid construct according to claim 1, wherein one of the first or second CARs is an activating CAR comprising an activating endodomain and the other CAR is an inhibitory CAR comprising a ligation-off inhibitory endodomain, which inhibitory CAR inhibits T-cell activation by the activating CAR in the absence of inhibitory CAR ligation but does not significantly inhibit T-cell activation by the activating CAR when the inhibitory CAR is ligated.

5. The nucleic acid construct according to claim 4, wherein the inhibitory endodomain comprises all or part of the endodomain from CD148 or CD45.

6-11. (canceled)

12. The nucleic acid construct according to claim 1 wherein the first and/or second CAR comprises an intracellular retention signal which directs the CAR to a lysozomal, endosomal or Golgi compartment.

13. The nucleic acid construct according to claim 1 wherein the first and/or second CAR comprises an intracellular retention signal selected from the following group: an endocytosis signal; a Golgi retention signal; a trans-Golgi network (TGN) recycling signal; an endoplasmic reticulum (ER) retention signal; and a lysosomal sorting signal.

14-20. (canceled)

21. The nucleic acid construct according to claim 1, wherein the signal peptide of the first or second CAR comprises one or more mutation(s) such that it has fewer hydrophobic amino acids than a) its wild-type sequence or b) the signal peptide of the other CAR.

22. The nucleic acid construct according to claim 21, wherein the signal peptide of the activating CAR comprises one or more mutation(s) such that it has fewer hydrophobic amino acids, such that when the nucleic acid construct is expressed in a cell, the relative expression level of the activating CAR at the cell surface is reduced in comparison with the inhibitory CAR.

23. The nucleic acid construct according to claim 21, wherein the hydrophobic amino acid(s) removed or replaced by mutation is/are selected from: Alanine Valine (A), Valine (V), Isoleucine (I), Leucine (L), Methionine (M), Phenylalanine (P), Tyrosine (Y), and Tryptophan (W).

24. The nucleic acid construct according to claim 21, wherein the hydrophobic amino acid(s) removed or replaced by mutation is/are selected from: Valine (V), Isoleucine (I), Leucine (L), and Tryptophan (W).

25. The nucleic acid construct according to claim 1, wherein the signal peptide of the first and second CAR differ in their number of hydrophobic amino acids and wherein one signal peptide comprises up to five more hydrophobic amino acids than the other signal peptide.

26. A vector comprising a nucleic acid construct according to claim 1.

27. (canceled)

28. A cell comprising a nucleic acid construct according to claim 1.

29. The cell according to claim 28 which is a T cell or a natural killer (NK) cell.

30. A method for making a cell according to claim 28, which comprises the step of introducing into a cell: a nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(i) an antigen-binding domain,
(ii) a spacer,
(iii) a trans-membrane domain, and
(iv) an endodomain
wherein the antigen binding domains of the first and second CARs bind to different antigens, wherein the spacer of the first CAR is different to the spacer of the second CAR, wherein one of the first or second CARs is an activating CAR comprising an activating endodomain and the other CAR is an inhibitory CAR comprising a ligation-off inhibitory endodomain, and wherein:
(a) the first and/or second CAR comprises an intracellular retention signal; and/or
(b) the signal peptide of the first or second CAR comprises one or more mutation(s) such that it has fewer hydrophobic amino acids.

31. The method according to claim 30, wherein the cell is from a sample isolated from a subject.

32. A pharmaceutical composition comprising a plurality of cells according to claim 28.

33. A method for treating and/or preventing a disease, which comprises the step of administering a pharmaceutical composition according to claim 32 to a subject.

34. The method according to claim 33, comprising the following steps:
(i) isolating a T and/or NK cell-containing sample from a subject;
(ii) transducing or transfecting the T and/or NK cells with: a nucleic acid construct comprising the following structure:
A-X-B
in which
X is a nucleic acid sequence which encodes a cleavage site, and
A and B are nucleic acid sequences encoding a first and a second chimeric antigen receptor (CAR), each CAR comprising:
(i) an antigen-binding domain,
(ii) a spacer,
(iii) a trans-membrane domain, and
(iv) an endodomain
wherein the antigen binding domains of the first and second CARs bind to different antigens, wherein the spacer of the first CAR is different to the spacer of the second CAR, wherein one of the first or second CARs is an activating CAR comprising an activating endodomain and the other CAR is an inhibitory CAR comprising a ligation-off inhibitory endodomain, and wherein:
(a) the first and/or second CAR comprises an intracellular retention signal; and/or
(b) the signal peptide of the first or second CAR comprises one or more mutation(s) such that it has fewer hydrophobic amino acids; and
(iii) administering the T and/or NK cells from (ii) to a the subject.

35. The method according to claim 33, wherein the disease is a cancer.

36-37. (canceled)
